(19) United States
(12) Patent Application Publication
(10) Pub. No.: US 2004/0077667 A1
(43) Pub. Date: Apr. 22, 2004

(54) Quinazolinone derivatives

(76) Inventors: Nobuya Matsuoka, Osaka-shi (JP); Akinori Iwashita, Osaka-shi (JP); Shunji Yamazaki, Osaka-shi (JP); Hiroshi Miyake, Osaka-shi (JP); Mitsuru Ohkubo, Osaka-shi (JP); Kazunori Kamijo, Osaka-shi (JP); Isao Nakanishi, Osaka-shi (JP); Kenji Murano, Osaka-shi (JP); Kouji Hattori, Osaka-shi (JP); Yoshiyuki Kido, Osaka-shi (JP); Junya Ishida, Osaka-shi (JP); Hirofumi Yamamoto, Osaka-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/433,947

(22) PCT Filed: Dec. 5, 2001
(86) PCT No.: PCT/JP01/10601

(30) Foreign Application Priority Data
Dec. 11, 2000 (AU) PR2016

(51) Int. Cl. A61K 31/517; C 07D 4 3/02

(52) U.S. Cl. 514/266.2; 544/284

(57) ABSTRACT

A quinazolinone derivatives having poly (adenosine 5′-diphaspho-ribose)polymerase (PARP) inhibotory activity represented by the formula (I), wherein R1 is optionally substituted cyclic amino groups or optionally substituted amino group, R2 is substituent, n means an integer from 0 to 4, and L is lower akkylene or lower alkenylene, or its prodrug, or their salts.

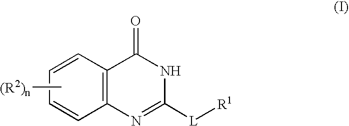

### TECHNICAL FIELD

This invention relates to novel quinazolinone derivatives having pharmacological activity, to a process for their production and to a pharmaceutical composition containing the same.

### BACKGROUND ART

Poly(adenosine 5′-diphaspho-ribose)polymerase [“poly(ADP-ribose)polynerase” or “PARP”, which is also sometimes called “PARS” for “poly(ADP-ribose)synthetase”] is an enzyme located in the nuclei of cells of various organs, including muscle, heart and brain cells. PARP plays a physiological role in the repair of strand breaks in DNA. Once activated by damaged DNA fragments, PARP catalyzes the attachment of up to 100 ADP-ribose units to a variety of nuclear proteins, including histones and PARP itself.

Some quinazolinone derivatives having inhibitory activity of PARP have been known, for example, in WO95/24379, WO98/33802 and WO99/11624.

### DISCLOSURE OF THE INVENTION

This invention relates to novel quinazolinone compounds, which have pharmaceutical activity such as PARP inhibiting activity, to a process for their production, to a pharmaceutical composition containing the same and to a use thereof.

One object of this invention is to provide the novel quinazolinone compounds, which have a PARP inhibiting activity.

Another object of this invention is to provide a process for production of the quinazolinone compounds.

A further object of this invention is to provide a pharmaceutical composition containing the quinazolinone compound as an active ingredient.

Still further object of this invention is to provide a use of the quinazolinone compound for manufacturing a medicament for treating or preventing various diseases, or a method of treating or preventing various diseases by administering the quinazolinone compound in an effective amount to inhibit PARP activity.

Thus, the present invention provides the following.

[1] A compound of the formula:

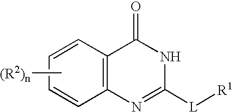

wherein

R¹ is optionally substituted cyclic amino groups or optionally substituted amino group,

R² is substituent,

n means an integer from 0 to 4, and

L is lower alkylene or lower alkenylene, or its prodrug, or their salts.

[2] The compound according to [1], wherein

R² is halogen, nitro, amino, acylamino, aryl(lower)alkylamino, lower alkylamino, lower alkyl, lower alkynyl, lower alkoxy, acyl, or cyclic amino group optionally substituted with lower alkyl.

[3] The compound according to [2], wherein

R¹ is (1) cyclic amino group optionally substituted with one or more substituent(s) selected from the group consisting of halogen, cyano, hydroxy, amino, oxo, lower alkyl, lower alkenyl, lower alkynyl, aryl(lower)alkyl, aryl(lower)alkynyl, acyl, lower alkylsulfonyl, optionally substituted heteroaryl and optionally substituted aryl, or (2) amino optionally substituted with 1 or 2 substituent(s) selected from the group consisting of lower alkyl, aryl, heteroaryl(lower)alkyl, aryl(lower)alkoxycarbonyl and aryl(lower)alkyl optionally substituted with aryl or aryloxy.

[4] The compound according to [3], wherein

R¹ is cyclic amino group optionally substituted with optionally substituted heteroaryl or optionally substituted aryl.

[5] The compound according to [4], wherein

R¹ is cyclic amino group with saturated or unsaturated monocyclic group with one or more nitrogen atom(s), which is substituted with optionally substituted heteroaryl or optionally substituted aryl.

[6] The compound according to [5], wherein

R¹ is tetrahydropyridyl, piperidyl or piperazinyl, each of which is substituted with optionally substituted heteroaryl or optionally substituted aryl.

[7] The compound according to any one of [4], [5] and [6], wherein

substituent(s) of optionally substituted heteroaryl is lower alkyl, halogen, cyano or acyl, or

substituent(s) of optionally substituted aryl is halogen, cyano, hydroxy, carboxy, nitro, amino, lower alkyl, hydroxy(lower)alkyl, lower alkoxy, lower alkyl thio, halo(lower)alkyl, lower alkylamino, acylamino, halo(lower)alkoxy, aryl, aryloxy, or acyl.

[8] The compound according to [3], wherein

R¹ is cyclic amino groups with saturated and unsaturated fused cyclic groups, which is substituted with optionally substituted lower alkyl.

[9] The compound according to any one of [4], [5], [6], [7] and [8], wherein

L is trimethylene.

[10] The compound according to [9], which is selected from the group consisting of:

(1) 5-chloro-2-[3-(4-phenyl-3,6-dihydro-1(2H)-pyridinyl)propyl]-4(3H)-quinazolinone,

(2) 2-{3-[4-(4-hydroxyphenyl)-3,6-dihydropyridin-1(2H)-yl]propyl}-4(3H)-quinazolinone,

(3) 8-methyl-2-{3-[4-(4-methoxyphenyl)-3,6-dihydro-1(2H)-pyridinyl]propyl}-4(3H)-quinazolinone,

(4) 8-chloro-2-{3-[4-(4-fluorophenyl)-3,6-dihydro-1(2H)-pyridinyl]propyl}-4(3H)-quinazolinone,

(5) 8-chloro-2-{(1E)-3-[4-(4-fluorophenyl)-3,6-dihydro-1(2H)-pyridinyl]-1-propenyl}-4(3H)-quinazolinone,

(6) 8-Chloro-2-{[4-(4-pyridinyl)-3,6-dihydro-1(2H)-pyridinyl]propyl}-4(3H)-quinazolinone,

(7) 2-{3-[4-(4-chlorophenyl)-1-piperazinyl]propyl}-4(3H)-quinazolinone,

(8) 2-{3-[4-(4-pyridyl)-1-piperazinyl]propyl}-4(3H)-quinazolinone,

(9) 2-[3-(1,4,5,6-Tetrahydrobenzo[f]isoquinolin-3(2H)-yl)propyl]-4(3H)-quinazolinone and

(10) 8-methyl-2-[3-(1,3,4,9-tetrahydro-2H-pyrido[3,4-b]indol-2-yl)propyl]-4(3H)-quinazolinone.

[11] A process for preparing a compound of the formula:

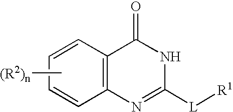

wherein R¹ is optionally substituted cyclic amino groups or optionally substituted amino group,

R² is substituent,

n means an integer from 0 to 4, and

L is lower alkylene or lower alkenylene, or its prodrug, or their salts, which comprises,

(1) reacting the formyl group of the compound (II) of the formula:

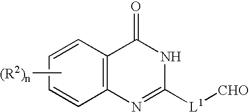

or its aminal derivative, or their salt, and imino group of the compound (IV) of the formula:

R¹—H

or its salt, in the presence of a reducing agent to provide a compound of the formula:

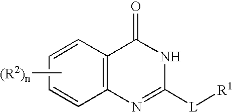

or its salt, in the above formulae,

R¹, R², n and L are each as defined above, and L¹ is lower alkylene or lower alkenylene delating a methylene group from the end of the one defined in L, or

(2) subjecting the compound (III) of the following formula:

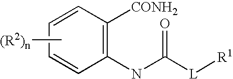

or its salt, to cyclization reaction in the presence of base to provide a compound of the formula:

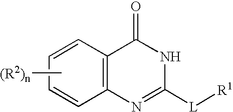

or its salt, in the above formurae,

R¹, R², n and L are each as defined above.

[12] A pharmaceutically composition comprising a compound of the formula:

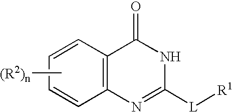

wherein

R¹ is optionally substituted cyclic amino groups or optionally substituted amino group,

R² is substituent,

n means an integer from 0 to 4, and

L is lower alkylene or lower alkenylene, or its prodrug, or their pharmaceutically acceptable salts, and a pharmaceutically acceptable carrier, wherein said compound is present in an amount effective for inhibiting PARP activity.

[13] The pharmaceutical composition of [12] for treating or preventing diseases ascribed by NMDA- and NO-induced toxicity.

[14] The pharmaceutical composition of [12] for extending the lifespan or proliferative capacity of cells or altering gene expression of senescent cells

[15] The pharmaceutical composition of [13] for treating or preventing tissue damage resulting from cell damage or death due to necrosis or apoptosis; neural tissue damage resulting from ischemia and reperfusion injury, neurological disorders and neurodegenerative diseases; neurodegenerative diseases; head trauma; stroke; Alzheimer's disease; Perkinson's disease, epilepsy; Amyotrophic Lateral Scleosis (ALS); Huntington's disease; schizopherenia; chronic pain; ischemia and nloss following hypoxia; hypoglycemia; ischemia; trauma; nervous insult; previously ischemic heart or skeleton muscle tissue; radiosensitizing hypoxic tumor cells; tumor cells from recovering from potentially lethal damage of DNA after radiation therapy; skin aging; atheroscleosis; osteoarthritis; osteoporosis; muscular dystrophy; degenerative diseases of skeletal muscle involving replicative senescence; age-related macular degeneration; immune senescence; AIDS; and other immune senescencediseases; inflammatory bowel disorders (e.g., colitis); arthritis; diabetes; endotoxic shock; septic shock; and tumor.

[16] A method of inhibiting PARP activity comprising administering a compound of the formula:

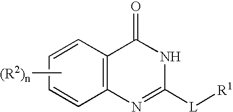

wherein

R¹ is optionally substituted cyclic amino groups or optionally substituted amino group,

R² is substituent,

n means an integer from 0 to 4, and

L is lower alkylene or lower alkenylene,

or its prodrug, or their pharmaceutically acceptable salts, and a pharmaceutically acceptable carrier, wherein said compound is present in an amount effective for inhibiting PARP activity.

The quinazolinone compounds of this invention can be represented by the following formula (I):

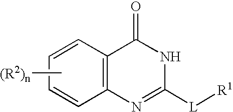

[wherein R¹ is optionally substituted cyclic amino groups or optionally substituted amino group, R² is substituent, n means an integer from 0 to 4, and L is lower alkylene or lower alkenylene] or its prodrug, or their salt.

The compound (I) or its prodrug, or their salt can be prepared by the following processes. In the following formulae, compounds may be prodrugs or their salts.

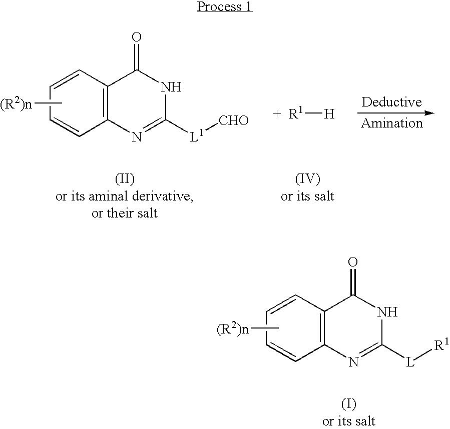

[wherein, R¹, R², n and L are each as defined above, and L¹ is lower alkylene or lower alkenylene delating a methylene group from the end of the lower alkylene defined in L]

In this process the compound (I) can be produced by reacting the formyl group of the compound (II) and imino or amino group of the compound (IV) in the presence of a reducing agent such as sodium cyanoborohydride, sodium borohydride, lithium cyanoborohydride, borane, diethylsilane, catalytic reduction with Raney nickel, or the like. This reaction preferably carried out in the acidic condition, such as the presence of acid (e.g., acetic acid, hydrogen chloride, trifluoroacetic acid).

The reaction is usually carried out in a conventional solvent such as water, an alcohol (e.g., methanol, ethanol or isopropyl alcohol), ether (e.g., tetrahydrofuran, dioxane, diethylether), amide (e.g., N,N-dimethylformamide, N,N-dimethylacetamide), nitrile (e.g., acetonitrile), or any other organic solvent which does not adversely affect the reaction. The reaction may be usually carried out under cooling to heating since the reaction temperature is not critical.

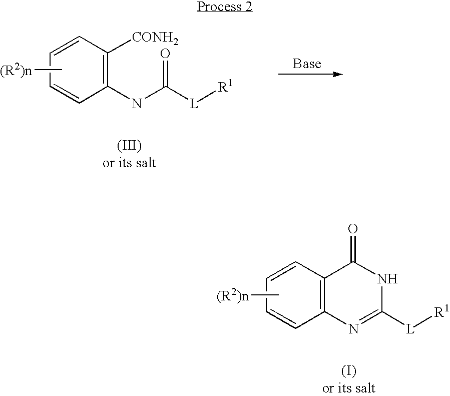

[wherein, R¹, R², n and L are each as defined above.]

In this process, the compound (I) can be produced by subjecting the compound (III) to cyclization reaction in the presence of base, such as inorganic bases, for example, an alkali metal [e.g., sodium or potassium], alkoxide, hydroxide, carbonate or bicarbonate thereof, or organic bases such as a trialkylamine [e.g., trimethylamine or triethylamine] or the like.

The reaction is usually carried out in a conventional solvent such as water, an alcohol (e.g., methanol, ethanol or isopropyl alcohol), ether (e.g., tetrahydrofuran, dioxane, diethylether), amide (e.g., N,N-dimethylformamide, N,N-dimethylacetamide), nitrile (e.g., acetonitrile), or any other organic solvent which does not adversely affect the reaction. The reaction may be usually carried out under cooling to heating since the reaction temperature is not critical.

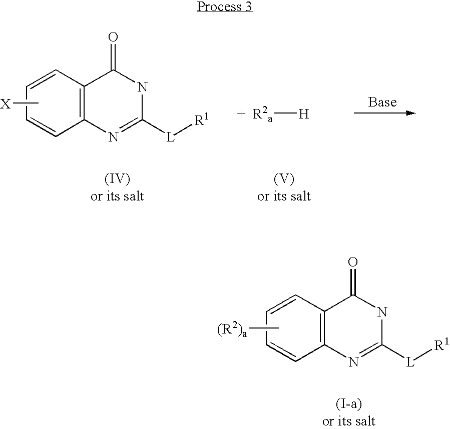

[wherein, X is leaving group, R²ₐ is cyclic amino group, R¹, n and L are each as defined above.]

In this process, the compound (I-a) or its salts can be produced by reacting the compound (IV) or its salt and compound (V) in the presence of base, such as inorganic bases, for example, an alkali metal [e.g., sodium or potassium], alkoxide, hydroxide, carbonate or bicarbonate thereof, or organic bases such as a trialkylamine [e.g., trimethylamine or triethylamine] or the like.

The reaction is usually carried out in a conventional solvent such as an alcohol (e.g., methanol, ethanol or isopropyl alcohol), ether (e.g., tetrahydrofuran, dioxane, diethylether), amide (e.g., N,N-dimethylformamide, N,N-dimethylacetamide), nitrile (e.g., acetonitrile), or any other organic solvent which does not adversely affect the reaction.

The reaction may be usually carried out under cooling to heating since the reaction temperature is not critical.

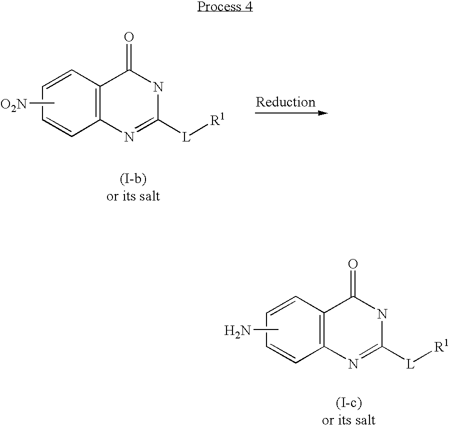

[wherein, R¹, n and L are each as defined above.]

In process 4, the compound (I-c) or its salt can be prepared by subjecting a compound (I-b) or its salt to reduction.

The reduction is carried out by chemical reduction, catalytic reduction, or the like. Suitable reducing agents to be used in chemical reduction are a combination of metal [e.g. tin, zinc, iron, etc.] or metallic compound [e.g. chromium chloride, chromium acetate, etc.] and an organic or inorganic acid [e.g. formic acid, acetic acid, propionic acid, trifluoroacetic acid, p-toluenesulfonic acid, hydrochloric acid, hydrobromic acid, etc.]. Suitable catalysts to be used in catalytic reduction are conventional ones such as platinum catalyst [e.g. platinum, platinum black, platinum oxide, etc.], palladium catalyst [e.g. palladium black, palladium oxide, palladium on carbon, etc.], nickel catalyst [e.g. reduced nickel, nickel oxide, Raney nickel, etc.], or the like.

The reduction is usually carried out in a conventional solvent which does not adversely influence the reaction such as water, an alcohol [e.g. methanol, ethanol, propanol, etc.], N,N-dimethylformamide, or a mixture thereof Additionally, in case that the above-mentioned acids to be used in chemical reduction are in liquid, they can also be used as a solvent. The reaction temperature of this reduction is not critical and the reaction is usually carried out under cooling to warming.

The compound of the present invention can be purified by any conventional purification methods employed for purifying organic compounds, such as recrystallization, column chromatography, thin-layer chromatography, high-performance liquid chromatography and the like. The compounds can be identified by conventional methods such as NMR spectrography, mass spectrography, IR spectrography, elemental analysis, and measurement of melting point.

Some of the starting compounds (II) or (III) are novel and can be prepared by the well-known processes or its analogous processes, for example, the processes described in the J. Med. Chem. 1998, 41, 5247-5256 and J. Org. Chem., 21, 478-(1956). The following processes are given as an example.

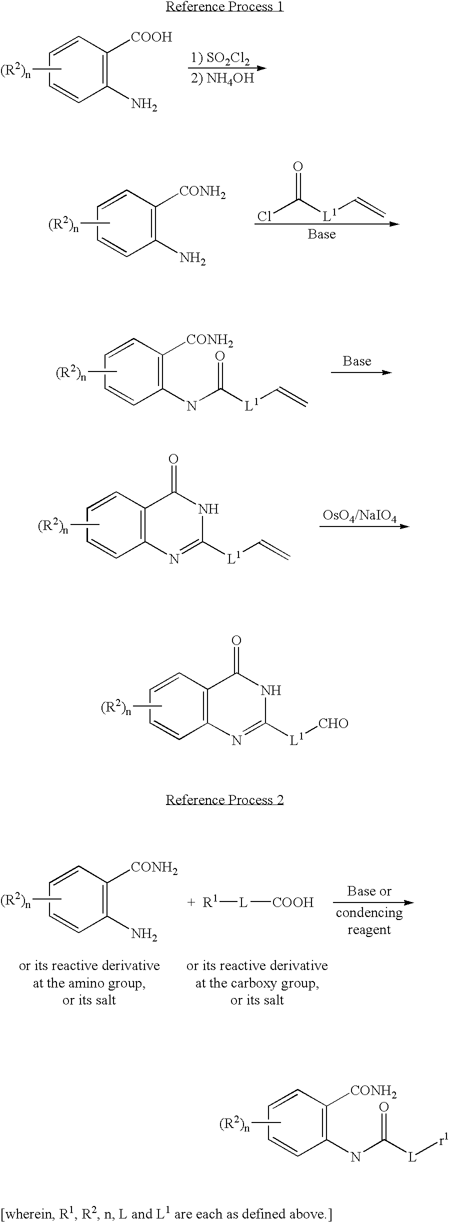

Suitable salts of the compounds of the present invention are pharmaceutically acceptable conventional non-toxic salts and can be an organic acid addition salt (e.g. formate, acetate, trifluoroacetate, maleate, tartarate, oxalate, methanesulfonate, benzenesulfonate, toluenesulfonate, etc.), an inorganic acid addition salt (e.g. hydrochloride, hydrobromide, sulfate, phosphate, etc.), a salt with an amino acid (e.g. aspartic acid salt, glutamic acid salt, etc.), or the like.

The “prodrug” means the derivatives of compounds of the present invention having a chemically or metabolically degradable group, which becomes pharmaceutically active after biotransformation.

The compounds of formula (I) may contain one or more asymmetric centers and thus they can exist as enantiomers or diastereoisomers. Furthermore certain compounds of formula (I) which contain alkenyl groups may exist as cis- or trans-isomers. In each instance, the invention includes both mixtures and separate individual isomers.

The compounds of the formula (I) may also exist in tautomeric forms and the invention includes both mixtures and separate individual tautomers.

The compound of the formula (I) and its salt can be in a form of a solvate, which is included within the scope of the present invention. The solvate preferably include a hydrate and an ethanolate.

Also included in the scope of invention are radiolabelled derivatives of compounds of formula (I) which are suitable for biological studies.

In the above and subsequent description of the present specification, suitable examples and illustrations of the various definitions, which the present invention includes within the scope thereof, are explained in detail as follows.

The term “lower” means a group having 1 to 6 carbon atom(s), unless otherwise provided.

Suitable “lower alkyl” and lower alkyl moiety in the terms “hydroxy(lower)alkyl”, “lower alkylsulfonyl”, “lower alkylthio” and “heteroaryl(lower)alkyl” include a straight or branched alkyl having 1 to 6, in particular 1 to 2, carbon atoms. Preferable examples which may be mentioned are methyl, ethyl, propyl, isopropyl, butyl, isobutyl, tert-butyl, pentyl and hexyl.

Preferable example which may be mentioned as “hydroxy(lower)alkyl” is hydroxymethyl. Preferable examples which may be mentioned as “lower alkylsulfonyl” are methylsulfonyl and ethylsulfonyl. Preferable examples which may be mentioned as “lower alkylthio” are methylthio and ethylthio.

Suitable “lower alkenyl” includes a straight or branched alkenyl having 2 to 6 carbon atoms. Preferable xamples which may be mentioned are ethenyl(vinyl), propenyl (i.e., allyl or 1-propenyl), butenyl and isobutenyl.

Suitable “lower alkynyl” and lower alkynyl moiety in the term “aryl(lower)alkynyl” include a straight or branch alkynyl having 2 to 6 carbon atoms. Preferable examples which may be mentioned are ethynyl and propynyl.

Preferable example which may be mentioned as “aryl(lower)alkynyl” is phenylethynyl.

Suitable “lower alkylene” includes a straight or branched alkylene having 1 to 6, in particular 3, carbon atoms. Preferable examples which may be mentioned are methylene, ethylene, trimethylene, propylene, methyltrimethylene (1- or 2-methyltrimethylene) and hexamethylene, preferably trimethylene.

Suitable “lower alkenylene” includes a straight or branched alkenylene having 1 to 6, in particular 3, carbon atoms. Preferable examples which may be mentioned are vinylene, propenylene, dimethylpropenylene (e.g., 3,3-dimethylpropenylene, etc.) and hexenylene preferably propenylene.

Suitable “lower alkoxy” and lower alkoxy moiety in the term “aryl(lower)alkoxycarbonyl” includes straight or branched alkoxy having 1 to 6, in particular 1 to 2, carbon atoms. Preferable examples which may be mentioned are methoxy, ethoxy, n-propoxy, isopropoxy, n-butoxy, iso-butoxy, sec-butoxy and tert-butoxy, preferably methoxy. Suitable “lower alkylamino” and lower alkylamino moiety in the term

“aryl(lower)alkylamino” include mono(lower)alkylamino and di(lower)alkylamino. Preferable examples which may be mentioned are methylamino, dimethylamino, ethylamino, dimethylamino, n-propylamino, isopropylamino, n-butylamino, iso-butylamino, sec-butylamino and tert-butylamino, preferably dimethylamino and diethylamino.

Suitable “aryl” and aryl moiety in the terms “aryloxy”, “aryl(lower)alkynyl”, “aryl(lower)alkylamino” and “aryl(lower)alkoxycarbonyl” may be intended to mean a mono-, di- or polynuclear aromatic radical having preferably 6 to 12 carbon atoms, such as phenyl, naphthyl, tetrahydronaphthyl, indenyl, indanyl (1,2-dihydroindenyl), fluorenyl and the like, preferably phenyl or naphthyl.

Preferable examples which may be mentioned as “aryloxy” are phenoxy and naphtyloxy.

Preferable example which may be mentioned as “aryl(lower)alkoxycarbonyl” is benzyloxycarbonyl.

Suitable “aryl(lower)alkyl” and aryl(lower)alkyl moiety in the term “aryl(lower)alkylamino” means arylalkyl which has preferably 6 or 10 carbon atoms in the aryl part (preferably phenyl or naphthyl, in particular phenyl) and preferably 1 to 6, in particular 1 to 4, carbon atoms in the alkyl part, it being possible for the alkyl part to be straight-chain or branched. Benzyl, 2-phenylethyl, 3-phenylpropyl, 4-phenylbutyl and naphtylmethyl may be mentioned as examples and as preferred.

Preferable examples which mentioned as “aryl(lower)alkylamino” are benzylamino and phenetylamino.

Suitable “acyl” and acyl moiety in the “acylamino” may be aliphatic acyl, aromatic acyl, aliphatic acyl optionally substituted aryl or heteroaromatic acyl, which are derived from carboxylic acid.

The aliphatic acyl may include

(1) lower alkanoyl optionally substituted with one or more suitable substituent(s) such as hydroxy, lower alkoxy, carboxy, protected carboxy, halogen, lower alkylthio, heterocyclicthio, oxo, cyclo(lower)alkyl or a heterocyclic group (e.g. formyl, acetyl, propionyl, butyryl, isobutyryl, pentanoyl, hexanoyl, 3,3-dimethylbutanoyl, 3-hydroxy-3-methylbutanoyl, 3-oxo-butanoyl, 3-methoxycarbonylpropanoyl, 3-carboxypropanoyl, 4-methoxycarbonylbutanoyl, 4-carboxybutanoyl, methylthioacetyl, (1-methylimidazol-2-yl)thioacetyl, hydroxyacetyl, methoxyacetyl, ethoxyacetyl, 3-methoxybutanoyl, chloroacetyl, morpholinoacetyl, piperidinylacetyl, 4-methylpiperidin-1-ylacetyl, 4-hydroxypiperidinyl, pyrolidinylacetyl, 4-(pyrimidin-2-yl)piperidinylacetyl, 3-hydroxypyrrolidinylacetyl, oxolan-4-ylacetyl, and so on);

(2) cyclo(lower)alkanecarbonyl (e.g. cyclopropylcarbonyl, cyclobutylcarbonyl, cyclopentylcarbonyl, cyclohexylcarbonyl, and so on);

(3) lower alkenoyl (e.g. acryloyl, methacryloyl, crotonoyl, 3-methylbutanoyl, and so on);

The aromatic acyl may include aroyl optionally substituted with one or more suitable substituent(s) such as nitro (e.g. benzoyl, naphthoyl, nitrobenzoyl, and so on), or the like.

The aliphatic acyl substituted with aryl may include ar(lower)alkanoyl which may have one or more suitable substituent(s) such as lower alkoxy (e.g. phenylacetyl, 4-methoxyphenylacetyl, and so on) or the like.

The heteroaromatic acyl is a carbonyl group to which is binded to heteroaryl, such as furylcarbonyl or the like.

The term “halogen” means fluoro, chloro, bromo or iodo.

Suitable “halo(lower)alkyl” and halo(lower)alkyl moiety in the term “halo(lower)alkoxy” contains 1 to 4, in particular 1 or 2, carbon atoms, and preferably 1 to 9, in particular 1 to 5, identical or different halogen atoms, preferably fluorine, chlorine and bromine, in particular fluorine and chlorine. Examples which may be mentioned are trifluoromethyl, trichloromethyl, chlorodifluoromethyl, dichlorofluoromethyl, chloromethyl, bromomethyl, 1-fluoroethyl, 2-fluoroethyl, 2,2-difluoroethyl, 2,2,2-trifluoroethyl, 2,2,2-trichloroethyl and pentafluoroethyl, preferably trifluoromethyl.

Suitable “heteroaryl” and heteroaryl moiety in the terms “heteroaryl(lower)alkyl” and “heteroaromatic acyl” is intended to mean 5- to 7-membered rings having preferably 1 to 3, in particular 1 or 2, identical or different heteroatoms. Heteroatoms in the heteroaryl are oxygen, sulfur or nitrogen. Examples which may be mentioned are furyl, thienyl, pyrazolyl, imidazolyl, triazolyl (e.g., 1,2,3- and 1,2,4-triazolyl, etc.), isoxazolyl, thiazolyl, isothiazolyl, oxadiazolyl (e.g., 1,3,4-, and 1,2,5-oxadiazolyl, etc.), azepinyl, pyrrolyl, pyridinyl, piperazinyl, pyridazinyl, pyrimidinyl, pyrazinyl, triazinyl (e.g., 1,3,5-, 1,2,4- and 1,2,3-triazinyl, etc.), oxazinyl (e.g., 1,2,4- and 1,2,6-oxazinyl, etc.), oxepinyl, thiepinyl and diazepinyl (e.g., 1,2,4-diazepinyl, etc.), preferably thienyl, pyrazolyl, imidazolyl, thiazolyl, pyridinyl and pyrazinyl.

Suitable “cyclic amino group” are heteroaromatic or aliphatic ring systems having one or more nitrogen atoms as the heteroatom, in which the heterocyclic rings can be saturated or unsaturated, can be one ring system or several fused ring systems, and optionally contain further heteroatoms, suchas nitrogen, oxygen and sulfur and the like. Cyclic amino groups can furthermore also denote a spiro ring or a bridged ring system. The number of atoms which form cyclic amino groups is not limited, for example in the case of a single-ring system, they comprise 3 to 8 atoms, and in the case of a three-ring system, they comprise 7 to 11 atoms.

Preferable examples of “cyclic amino group” are described as follows: (1) examples which may be mentioned of cyclic amino group with saturated monocyclic groups with one or more nitrogen atom(s) as the heteroatom are azetidinyl (3-azetidinyl), pyrrolidinyl (e.g., 1- and 3-pyrrolidinyl, etc.), piperidyl (e.g., 1- and 4-piperidyl, etc.), homopiperidino (e.g., hexahydro-1H-azepin-1-yl, etc.), homopiperazinyl (e.g., hexahydro-1H-1,4-diazepin-1-yl, etc.), imidazolidinyl (e.g., 1-imidazolidinyl, etc.), piperazinyl (e.g., 1-piperazinyl, etc.), perhydropyrimidinyl (e.g., perhydropyrimidin-1-yl, etc.) and diazacycloheptanyl (e.g., 1,4-diazacycloheptan-1-yl, etc.);

(2) examples which may be mentioned of cyclic amino group with unsaturated monocyclic groups with one or more nitrogen atom(s) as the heteroatom are pyrrolinyl (e.g., 2-pyrrolin-1-yl, etc.), pyrrolyl (e.g, 1-pyrrolyl, etc), tetrahydropridinyl (e.g., 3,6-dihydro-1(2H)-pyridinyl, etc.), pyridinyl (e.g., 2-pyridinyl, etc.), tetrahydroazepinyl (e.g., 2,3,6,7-tetrahydro-1H-azepin-1-yl, 2,3,4,7-tetrahydro-1H-azepin-1-yl, etc.), imidazolyl (1-imidazolyl), pyrazolyl, triazolyl, tetrazolyl, tetrazolyl, pyrimidinyl, pyrazinyl, pyridazinyl, dihydro-pyridazinyl (e.g., 1,2-dihydro-pyridazin-1-yl, etc.) and dihydro-pyrimidinyl (e.g., 1,2-dihydro-pyrimidin-1-yl, etc.);

(3) examples which may be mentioned of cyclic amino groups with saturated and unsaturated monocyclic groups with one to three nitrogen atoms and one to two sulfur atoms as heteroatoms are thiazolidinyl (e.g., 3-thiazolidinyl, etc.), isothiazolinyl (e.g., 2-isothiazolinyl, etc.) and thiomorpholino;

(4) examples which may be mentioned of cyclic amino groups with saturated and unsaturated monocyclic groups with one to three nitrogen atoms and one to two oxygen atoms as heteroatoms are oxazolyl, isoxazolyl, oxadiazolyl (e.g., 1,2,4-oxadiazolyl, and 1,3,4-oxadiazolyl) or morpholinyl;

(5) examples which may be mentioned of cyclic amino groups with saturated and unsaturated fused cyclic groups are indolyl (e.g., 1-indolyl, etc.), dihydrobenzimidazolyl (e.g., 1,2-dihydrobenzimidazol-1-yl, etc.), perhydropyrrolo[1,2-a]pyrazinyl (e.g., perhydropyrrolo[1,2-a]pyrazin-2-yl, etc.), tetrahydrobenzo[f]isoquinolinyl (e.g., 1,4,5,6-tetrahydrobenzo[f]isoquinolin-3(2H)-yl, etc.), hexahydrobenz[f]isoquinolinyl (e.g., cis- and trans-1,4,4a,5,6,10b-hexahydrobenz[f]isoquinolin-3(2H)-yl, etc.), tetrahydropyrido[3,4-b]indolyl (e.g., 1,3,4,9-tetrahydro-2H-pyrido[3,4b]indol-2-yl, etc.) tetrahydrobenzazepinyl (e.g., 1,2,4,5-tetrahydro-3H-3-benzazepin-3-yl, etc.) dihydroisoquinolinyl (e.g., 3,4-dihydro-2(1H)-isoquinolinyl, etc.);

(6) examples which may be mentioned of cyclic amino groups with spirocyclic groups are azaspiro[4,5]decanyl (e.g., 2-azaspiro[4,5]decan-2-yl, etc.), spiro[1H-indene-1,4′-piperidinyl] (e.g., spiro[1H-indene-1,4′-piperidin-1′-yl], etc.), and dihydrospiro[1H-indene-1,4′-piperidinyl] (e.g., 2,3-dihydrospiro[1H-indene-1,4′-piperidin-1′-yl], etc.);

(7) examples which may be mentioned of cyclic amino groups bridged heterocyclic groups are azabicyclo[2,2,1]heptanyl (e.g., 2-azabicyclo[2,2,1]heptan-7-yl, etc.) and diazabicyclo[2.2.1]heptyl (e.g., 2,5-diazabicyclo[2.2.1]hept-2-yl, etc.).

Among the above, preferable “cyclic smino group” included in R1 is above-mentioned (1) or (2), in which the most preferable one is piperidinyl, tetrahydropyridinyl and piperazinyl.

It has been known that, during major cellular stresses, the activation of PARP can rapidly lead to cell damage or death through depletion of energy stores and PARP activation play a key role in both NMDA- and NO-induced neurotoxicity (Zhang et. al., Science, 263: 687-89 (1994)). Therefore, the compound possessing PARP inhibiting activity, such as the compound (I) of this invention, or pharmaceutically acceptable salts are useful in treating and preventing various diseases ascribed by NMDA- and NO-induced toxicity. Such diseases include, for example, tissue damage resulting from cell damage or death due to necrosis or apoptosis; neural tissue damage resulting from ischemia and reperfusion injury, neurological disorders and neurodegenerative diseases; neurodegenerative diseases; head trauma; stroke; Alzheimer's disease; Perkinson's disease; epilepsy; amyotrophic lateral scleosis (ALS); Huntington's disease; schizophrenia; chronic pain; ischemia and neuronal loss following hypoxia; hypoglycemia; ischemia; trauma; and nervous insult.

It has been demonstrated that PARP inhibitor are useful in deducing infarct size (Thiemermann et al, Proc. Natl. Acad. Sci. USA, 94: 679-83 (1997)). Therefore, the compound possessing PARP inhibiting activity, such as the compound (I) of this invention, or pharmaceutically acceptable salts are useful in treatment and prevention of previously ischemic heart or skeleton muscle tissue.

It is also known that PARP is thought to play a role in enhancing DNA repair. So, the compound possessing PARP inhibiting activity, such as the compound (I) of this invention, or pharmaceutically acceptable salts are effective in treating and preventing radiosensitizing hypoxic tumor cells; tumor cells from recovering from potentially lethal damage of DNA after radiation therapy.

Further, the compound possessing PARP inhibiting activity, such as the compound (I) of this invention, or pharmaceutically acceptable salts are useful in extending the life-span and proliferative capacity of cells and altering gene expression of senescent cells. They are useful for treating and preventing skin aging; Alzheimer's diseases; atheroscleosis; osteoarthritis; osteoporosis; muscular dystrophy; degenerative diseases of skeletal muscle involving replicative senescence; age-related macular degeneration; immune senescence; AIDS; and other immune senescence diseases.

Still further, the compound possessing PARP inhibiting activity, such as the compound (I) of this invention, or pharmaceutically acceptable salts are effective in treating and preventing inflammatory bowel disorders (e.g., colitis); arthritis; diabetes; endotoxic shock; septic shock; and tumor. Also, they are useful in reducing proliferation of tumor cells and making synergistic effect when tumor cells are co-treated with an alkylating drug.

The compound possessing PARP inhibiting activity, such as the compound (I) of this invention, or pharmaceutically acceptable salts are effective in treating and preventing pituitary apoplexy; conjunctivitis; retinoblastoma; retinopathy; acute retinal necrosis syndrome; Sjogren's syndrome.

The compound (I), its prodrug, or their salt can be administered alone or in the form of a mixture, preferably, with a pharmaceutical vehicle or carrier.

The active ingredient of this invention can be used in the form of a pharmaceutical preparation, for example, in solid, semisolid or liquid form, which contains a compound (I), as an active ingredient, in admixture with an organic or inorganic carrier or excipient suitable for external (topical), enteral, intravenous, intramuscular, parenteral or intramucous applications. The active ingredient can be formulated, for example, with the conventional non-toxic, pharmaceutically acceptable carriers for ointment, cream, plaster, tablets, pellets, capsules, suppositories, solution (saline, for example), emulsion, suspension (olive oil, for example), aerosols, pills, powders, syrups, injections, troches, cataplasms, aromatic waters, lotions, buccal tablets, sublingual tablets, nasal drops and any other form suitable for use. The carriers which can be used are water, wax, glucose, lactose, gum acacia, gelatin, mannitol, starch paster, magnesium trisilicate, talc, corn starch, keratin, paraffin, colloidal silica, potato starch, urea and other carriers suitable for use in manufacturing preparations, in solid, semisolid, or liquid form, and in addition auxiliary, stabilizing, thickening and coloring agents and perfumes may be used. The active compound is included in a pharmaceutical composition in an effective amount sufficient to produce the desired effect upon the process or condition of the diseases.

The active ingredient can be formulated into, for example, preparations for oral application, preparations for injection, preparations for external application, preparations for inhalation, preparations for application to mucous membranes.

Mammals which may be treated by the present invention include livestock mammals such as cows, horses, etc., domestic animals such as dogs, cats, rats, etc. and humans, preferably humans.

While the dosage of therapeutically effective amount of the compound (I) will vary depending upon the age and condition of each individual patient, an average single dose to a human patient of about 0.01 mg, 0.1 mg, 1 mg, 10 mg, 50 mg, 100 mg, 250 mg, 500 mg, and 1000 mg of the compound (I) may be effective for treating the above-mentioned diseases. In general, amounts between 0.01 mg/body and about 1,000 mg/body may be administered per day.

In order to illustrate the usefulness of the object compound (I), the pharmacological test data of the compound (I) are shown in the following.

A. Test Compound

5-chloro-2-[3-(4-phenyl-3,6-dihydro-1(2H)-pyridinyl)propyl]-4(3H)-quinazolinone

(Compound A: The compound of Example 1)

8-chloro-2-{(1E)-3-[4-(4-fluorophenyl)-3,6-dihydro-1(2H)-pyridinyl]-1-propenyl}-4(3H)-quinazolinone,

(Compound B: The compound of Example 33 (1)) 8-Chloro-2-{[4-(4-pyridinyl)-3,6-dihydro-1(2H)-pyridinyl]propyl}-4(3H)-quinazolinone,

(Compound C: The compound of Example 35 (15)) 8-methyl-2-[3-(1,3,4,9-tetrahydro-2H-pyrido[3,4-b]indol-2-yl)propyl]-4(3H)-quinazolinone,

(Compound D: The compound of Example 38 (2))

B. PARP Inhibitory Activity (In Vitro Assay)

(1) Assay Conditions:

The recombinant human PARP (5.3 mg protein/ml) were incubated with a test compound in a 100 μl reaction buffer containing the indicated concentration of 1 mCi/ml ³²P-NAD, 50 mM Tris-HCl, 25 mM MgCl₂, 1 mM DTT (dithiothreitol), 0.05 mM NAD (nicotinamido adenine dinucleotide), 1 mg/ml activated DNA, pH8.0. Incubation was for 15 minutes at a room temperature and the reaction was stopped by the addition of 200 μl of ice-cold 20% tricholoroacetic acid followed by rapid filtration through GF/B filters. The filters were treated with scintillation fluid and acid-insoluble counts were measured for quantification of unit activity.

PARP inhibitory activity (%)=[1-(enzyme activity with test compound)/(enzyme activity with vehicle)]×100

(2) Result

PARP inhibitory activity (IC₅₀) in test compound

| Test Compound | IC50(μM) |
|---|---|
| Compound A | <0.5 |
| Compound B | <0.5 |
| Compound C | <0.5 |
| Compound D | <0.5 |

C. Effect of Test Compound on the Level of Striatal Dopamine and its Metabolite in Mice MPTP(N-methyl-1,2,3,6-tetrahydropyridine)-induced Parkinson's model

(1) Method

Mice received four i.p. injections of MPTP-HCl (20 mg/kg) in saline at 2 hours intervals and two i.p. injections of Test compound at 30 minutes before 1st injection and 3rd injection of MPTP.

Four days after the last MPTP injection, mice were sacrificed, brains were quickly removed, and striata were dissected out on an ice-cold glass Petri dish. Samples were homogenized in a buffer of 0.1M perchloric acid containing isoproterenol as internal standard. HPLC with electrochemical detection was used to measure striatal levels of of DA (dopamine), DOPAC (dihydroxyphenylacetic acid) and HVA (homovanilic acid).

(2) Results

The level of DA, DOPAC and HVA were expressed as a percentage of Normal taken as the 100%.

| | Dopamine levels |
|---|---|
| Normal | 100 |
| MPTP | 21 |
| MPTP + Compound A (32 mg/kg) | 59* |
| | DOPAC levels |
| Normal | 100 |
| MPTP | 25 |
| MPTP + Compound A (32 mg/kg) | 58* |
| | HVA levels |
| Normal | 100 |
| MPTP | 40 |
| MPTP + Compound A (32 mg/kg) | 64* |

*P < 0.05 vs MPTP (by Student's t-test)

This invention relates to novel Quinazoline compounds had a potent PARP inhibitory activity. PARP inhibitors including this invention relates to novel quinazoline compounds were effective in preventing reduction of striatal DA and its metabolite induced by MPTP treatment in mice. Therefore, it suggests that these compounds may have protective benefit in the treatment of neurodegenerative disease such as Parkinson's disease.

Abbreviations used herein have the following meanings:

| ABBREVIATION | DEFINITION |
|---|---|
| Me | methyl |
| Et | ethyl |
| tBu | tert-buthyl |
| Bzl | benzyl |
| Ph | phenyl |
| Ac | acetyl |
| Bz | benzoyl |

Any patents, patent applications, and publications cited herein are incorporated by reference.

### BEST MODE FOR CARRYING OUT THE INVENTION

The following Preparation and Examples are given for the purpose of illustrating the present invention in detail, but are not to be construed to limit the scope of the present invention.

Preparation 1

2-Amino-6-chlorobenzoic acid (150 g, 874 mmol) was added slowly to thionyl chloride (383 mL, 5.25 mol) at 5° C. and the mixture was refluxed for 2 hours. Thionyl chloride was removed in vacuo. Toluene was added and removed in vacuo. The obtained acid chloride was dissolved in dioxane (750 mL). The solution was added dropwise to NH₄OH (27%, 835 mL, 4.37 mol) at 5° C. The mixture was concentrated in vacuo. The reaction mixture was extracted with ethyl acetate. Hexane was added to the organic layer, and the precipitate was corrected with filtration. The resulting crystals were dried to give 2-amino-6-chlorobenzamide (95.8 g, 577 mmol, 64%).

¹HNMR (300 MHz, CDCl₃, δ): 4.84 (2H, br.s), 5.97(1H, br.s), 6.20(1H, br.s), 6.60(1, d J=8.2 Hz), 6.73 (1H, d, J=8.0 Hz), and 7.07 (1H, t, J=8.1 Hz)

Mass (m/z): 171 (M⁺+1)

Preparation 2

To a mixture of 2-amino-6-chlorobenzamide (100 g, 586 mmol) and diisopropyl-ethylamine (123 mL, 703 mmol) in THF (1L) 4-pentenoyl chloride (74.4 mL, 674 mmol) was added dropwise at 5° C. The mixture was stirred for 30 minutes. Saturated sodium hydrogen carbonate aqueous solution was added and the precipitate was corrected by filtration and washed with water to give 2-chloro-6-(4-pentenoylamino)benzamide, which was used without further purification.

¹H NMR (300 MHz, CDCl₃δ): 2.47(4H, s), 5.03 (1H, dd, J=10.1 Hz, <1 Hz), 5.13 (1H, dt, J=7.9 Hz, <1 Hz), 5.85 (1H, m), 6.15(1H, br.s), 6.28(1H, br.s), 7.34 (1H, t, J=8.3 Hz), 7.16 (1H, d, J=9.1 Hz, 8.23 (1H, d, J=8.4 Hz), and 9.26 (1H, br.s).

Mass (m/z): 253 (M⁺+1)

Preparation 3

2-Chloro-6-(4-pentenoylamino)benzamide (148 g, 586 mmol) was dissolved in dioxane (1L), and 1N NaOH aqueous solution (1.17L) was added. The reaction mixture was stirred at room temperature for 2.5 hours. The reaction mixture was concentrated in vacuo, then the resulting solution was neutralized with 1N HCl aqueous solution. The precipitate was corrected with filtration and washed with ether to give 2-(3-butenyl)-5-chloro-4(3H)-quinazolinone (96.6 g, 0.41 mmol, 70% for two steps) as colorless crystals.

¹H NMR (300 MHz, CDCl₃, δ): 2.66 (2H, q, J=7.3 Hz), 2.87 (2H, t, J=7.6 Hz), 5.50 (1H, d, J=9.9 Hz), 5.15 (1H, d, J=17.3 Hz), 5.09 (1H, m), 7.45 (1H, m), and 7.66 (2H, m).

Mass (m/z): 235 (⁺+1)

Preparation 4

OsO₄(2.5% t-BuOH solution, 23.8 mL, 2.34 mmol) was added to 10% aqueous dioxane solution of 2-(3-butenyl)-5-chloro-4(3H)-quinazolinone (55 g, 234 mmol). After stirring for 10 minutes, NaIO₄ (110 g, 516 mmol) was added to the mixture. The mixture was stirred at room temperature for 4 hours. The reaction mixture was extracted with AcOEt, and washed with 10% NaS₂O₃ and brine. The organic layer was dried over MgSO₄ and the solvent was removed in vacuo. The residual yellow solid was purified by silica gel chromatography eluting with chloroform and methanol (100:1-100:2) to give 8-chloro-1-hydroxy-2,3-dihydropyrrolo[2,1-b]quinazoline-9(1H)-one (26.5 g, 110 mmol, 48%) was obtained as colorless powder.

¹H NMR (300 MHz, CDCl₃, δ): 2.22 (1H, m), 2.50 (1H, m), 3.04 (1H, m), 3.35 (1H, m), 4.36 (1H, br.s), 6.28 (1H, m), 7.46 (1H, m), and 7.59 (2H, m).

Mass (m/z): 237 (M⁺+1)

Preparation 5

Benzylchloride (3.25 mL, 28.2 mmmol) was added to the mixture of 4-phenyl-4-hydroxypiperidine and t-BuOK (3.17 g, 28.2 mmol) in t-butanol (70 mL), and the mixture was refluxed for 2 hours. Methanol (30 mL) was added to the mixture and inorganic solid was filtered off. The solution was concentrated in vacuo and extracted with AcOEt, washed with brine. Solvent was removed in vacuo, and the residual solid was washed with diisopropylether/hexane (1:10) to give 1-benzyl-4-hydroxy-4-phenylpiperidine (6.32 g, 23.6 mmol, 84%) as colorless powder.

¹H NMR (300 MHz, CDCl₃, δ): 1.74(2H, dm, J=14.1 Hz), 2.18 (2H, td, J=13.0 Hz, 4.4 Hz), 2.48 (2H, tm, J=13.0 Hz), 2.80 (2H, dm, J=11.1 Hz), 3,59 (2H, s), 7.23-7.30 (3H, m), 7.33-7.38 (5H, m), and 7.52 (2H, dm, J=7.9 Hz).

Mass (m/z): 268 (M⁺+1).

Preparation 6

Sulfuric acid (16.7 mL, 314 mmol) was added dropwise to dispersion of 1-benzyl-4-hydroxy-4-phenylpiperidine (6 g, 22.4 mmol) in acetonitrile (25.8 mL, 494 mmol) at 0° C., and the mixture was stirred at room temperature for 30 minutes. The reaction mixture was poured into cold water. The solution was adjusted to pH 9 with saturated sodium hydrogen carbonate aqueous solution and 1N NaOH aqueous solution. The mixture was extracted with AcOEt, washed with saturated sodium hydrogen carbonate aqueous solution and brine. Solvent was removed in vacuo. Residual colorless solid was washed with ether to give 4-acetoamide-1-benzyl-4-phenylpiperidine (5.8 g, 19.0 mmol, 84%) as colorless powder.

¹H NMR (300 MHz, CDCl₃, δ): 2.03 (3H, m), 2.12 (2H, m), 2.30 (4H, m), 2.80 (2H, d, J=12.2 Hz), 3.53 (2H, s), 5.53 (1H, br.s), and 7.4-7.18 (10H, m).

Mass (m/z): 309 (M⁺+1)

Preparation 7

4-Acetoamide-1-benzyl-4-phenylpiperidine (2.7 g, 8.75 mmol) was dissolved in 6N aqueous HCl (7.27 mL, 43.8 mmol) at 130° C. After the solution was cooled to room temperature, 1N NaOH aqueous solution was added. The reaction mixture was extracted with AcOEt, washed with saturated sodium hydrogen carbonate aqueous solution. The organic layer was dried over MgSO₄ and the solvent was removed in vacuo. The residual pale yellow oil was purified by silica gel chromatography eluting with chloroform and methanol (100:5-100:20) to give 4-amino-1-benzyl-4-phenylpiperidine (1.7 g, 6.38 mmol, 73%) as pale yellow oil.

¹H NMR (300 MHz, CDCl₃, δ): 1.70(2H, m), 2.20 (2H, m), 2.50 (2H, m), 2.71 (2H, m), 3.57 (2H, s), 7.25 (2H, m), 7.35 (6H, tm, J=7.6 Hz), and 7.52 (2H, dm, J=7.24 Hz).

Mass (m/z): 267 (M⁺+1)

Preparation 8

4-Amino-1-benzyl-4-phenylpiperidine (500 mg, 1.88 mmol) and HCO₂NH₄ (1.18 g, 18.8 mmol), and Pd-C (10%, 500 mg) were disperted in ethanol/H2O (10 mL/10 mL). The mixture was refluxed for 4 hours. Insoluble products were filtrated off, and the solvent was removed in vacuo. The residue was purified by reverse phase chromatography eluting by water to give 4-amino-4-phenylpiperidine (20 mg, 11.3 mmol, 13.7%) as colorless solid.

¹H NMR (300 MHz, CDCl₃, δ): 1.73 (2H, m), 2.16 (2H, m), 2.79 (2H, m), 3.02 (2H, m), 7.22 (1H, tm, J=7.3 Hz), 7.35 (2H, tm, J=8.0 Hz), and 7.51 (2H, tm, J=7.3 Hz).

Mass (m/z): 177 (M⁺+1)

Preparation 9

Oxalyl chloride was added to a solution of 4-(1-phenyl-4-piperidyl)-butanoic acid (200 mg, 0.809 mmol) in DMF (5 mL) under ice water bath, then the mixture was stirred for 1 hour.

To a solution of 2-carbamoylaniline (110 mg, 0.809 mmol) in DMF (5 mL) was added N-ethyldiisopropylamine (0.169 mL, 0.97 mmol) under ice water bath, then the previous soluiton was added dropwise. After stirring 2 hours at room temperature, the mixture was poured into ice water, extracted ethyl acetate twice, washed with saturated aqueous NaHCO₃ and brine, and dried over sodium sulfate. Evaporation of the solvent gave the residue, and purified by silica gel chromatography eluting with chloroform and methanol (20:1) to give 2-[4-(1-phenyl-4-piperidyl)-butanoylamino]benzamide (100 mg, 0.26 mmol, 34%) as a pale yellow powder.

¹H NMR (300 MHz, CDCl₃, δ): 1.26-1.50 (5H, m), 1.72-1.89 (4H, m), 2.42 (2H, t, J=7.5 Hz), 2.68 (2H, t, J=7.0 Hz), 3.66 (2H, d, J=7.0 Hz), 6.81 (1H, t, J=7.8 Hz, 6.94 (2H, d, J=7.8 Hz), 7.08 (1H, t, J=7.8 Hz), 7.24 (2H, d, J=7.8 Hz), 7.42-7.56 (2H, m), 8.67 (1H, d, J=7.8 Hz), 11.15 (1H, s)

Mass (m/z): 366 (M⁺)

Preparation 10-(1)

Under a nitrogen atmosphere, a solution of butyllithium (1.6 M in hexane, 10.8 mL) was added dropwise to a solution of 1-bromo-4-methoxybenzene (3.04 g, 16.3 mmol) in tetrahydrofuran (30 mL) at −78° C. The mixture was stirred at the temperature for 30 minutes, and a solution of tert-butyl 4-oxo-1-piperidinecarboxylate (2.7 g, 13.6 mmol) in tetrahydrofuran (20 mL) was added dropwise. The mixture was allowed to warm to −20° C. with stirring for 2 hours. The reaction was quenched by addition of saturated aqueous ammonium chloride, and the organic materials were extracted with ethyl acetate. The organic layer was washed with water and brine and dried over magnesium sulfate. Purification over silica gel chromatography gave tert-butyl 4-hydroxy-4-(4-methoxyphenyl)-1-piperidinecarboxylate (3.04 g, 73.0%) as oil.

¹H NMR (200 MHz, CDCl₃, δ): 1.48 (9H, s), 1.73 (2H, br d, J=12.0 Hz), 1.97 (2H, dt, J=12.5, 2.4 Hz), 3.24 (2H, br t, J=11.6 Hz), 3.81 (3H, s), 4.02 (2H, br d, J=9.8 Hz), 6.89 (2H, d, J=8.9 Hz), 7.39 (2H, d, J=8.9 Hz)

Mass (APCI+, 50V): 330.3 (M⁺+Na)

Preparation 10-(2)

Trifluoroacetic acid (7.6 mL, 98.9 mmol) was added to an ice-cooled solution of tert-butyl 4-hydroxy-4-(4-methoxyphenyl)-1-piperidinecarboxylate (3.04 g, 9.89 mmol) in dichloromethane (15 mL), and the mixture was stirred at 0° C. for 1 hour. Trifluoroacetic acid and dichloromethane were removed in vacuo, and the crude product was treated with ethyl acetate and aqueous sodium hydrogen carbonate. The organic layer was separated, and dried over sodium sulfate. The evaporated residue was treated with a solution of hydrogen chloride (4 M in ethyl acetate, 5 mL) in ice-cooled ethyl acetate (15 mL) for 1 hour to give 4-(4-methoxyphenyl)-1,2,3,6-tetrahydropyridine hydrochloride (1.63 g, 73.0%) as powder.

¹H NMR (200 MHz, DMSO-d₆, δ): 2.66 (2H, br), 3.27 (2H, br), 3.70 (2H, br), 3.76 (3H, s), 6.08 (1H, m), 6.94 (2H, d, J=8.8 Hz), 7.42 (2H, d, J=8.8 Hz), 9.29 (2H, br)

Mass (API-ES+): 190.4 (M⁺+H)

Preparation 11

Tert-butyl 4-hydroxy4-[4-(trifluoromethyl)phenyl]-1-piperidinecarboxylate was prepared in a similar procedure to that of Preparation 10-(1), which was used for the next step (Preparation 12).

Preparation 12

Methanesulfonyl chloride (3.44 mL, 44.4 mmol) was added dropwise to a solution of tert-butyl 4-hydroxy-4-[4-(trifluoromethyl)phenyl]-1-piperidinecarboxylate (includes tert-butyl 4-oxo-1-piperidinecarboxylate, 5.11 g) in triethylamine (20.6 mL) and dichloromethane (60 mL) at −78° C. 4-Dimethylaminopyridine (90 mg, 0.74 mmol) was added, and the mixture was allowed to warm to 0° C. and was stirred for 2 hours at 0° C.

Quenched with water, and the organic materials were extracted with chloroform. Solvents were removed in vacuo, and the residue was dissolved in dichloromethane (50 mL) and triethylamine (20 mL), and stirred for 2 days at room temperature. Quenched by the addition of water, and the product was extracted with CHCl₃. Purification over silica gel (hexane:ethyl acetate=10:1) gave tert-butyl 3,6-tetrahydro-4-[4-(trifluoromethyl)phenyl]-1(2H)-pyridinecarboxylate (3.57 g, 73.7%).

¹H NMR (200 MHz, CDCl₃, δ): 1.50 (9H, s), 2.53 (2H, m), 3.65 (2H, t, J=5.7 Hz), 4.10 (2H, q, J=2.8 Hz), 6.12 (1H, br), 7.46 (2H, d, J=8.4 Hz), 7.58 (2H, d, J=8.5 Hz)

Mass (API-ES): 350.3 (M⁺+Na)

Preparation 13

A solution of hydrogen chloride (4 M in ethyl acetate, 16.4 mL) was added to a solution of tert-butyl 3,6-tetrahydro-4-[4-(trifluoromethyl)phenyl]-1(2H)-pyridinecarboxylate (3.57 g, 10.9 mmol) in ethyl acetate (4 mL) at 0° C. The mixture was stirred for 1.5 hr at the temperature. Evaporated to dryness, and the residue was washed with ethyl acetate and diisopropyl ether to give 4-[4-(trifluoromethyl)phenyl]-1,2,3,6-tetrahydropyridine hydrochloride (2.61 g, 90.8%) as white powder.

¹H NMR (200 MHz, DMSO-d₆, δ): 2.73 (2H, br), 3.32 (2H, t, J=6.0 Hz), 3.78 (2H, m), 6.37 (1H, br), 7.70 (2H, d, J=8.9 Hz), 7.76 (2H, d, J=9.0 Hz), 9.38 (2H, br s)

Mass (API-ES): 228.3 (M⁺+H)

Preparation 14

Under a nitrogen atmosphere, a mixture of tert-butyl 4-(((trifluoromethyl)sulfonyl)oxy)-3,6-dihydro-1(2H)-pyridinecarboxylate (1.0 g, 3.02 mmol), 4-cyanophenylboronic acid (532 mg, 3.62 mmol), triethylamine (1.26 mL, 9.05 mmol) and tetrakis(triphenylphosphine)palladium (35 mg, 0.030 mmol) in N,N-dimethylformamide (15 mL) was stirred for 2 hours at 100° C. Quenched with water, and the product was extracted with ethyl acetate. Solvents were removed in vacuo (treated with toluene once azeotropically) to give the crude product. It was treated with a solution of hydrogen chloride (4 M in ethyl acetate, 5 mL) in ice-cooled ethyl acetate (7 mL) for 1 hour. The precipitate was collected by filtration and washed with ethyl acetate and diisopropyl ether to give 4-(1,2,3,6-tetrahydro-4-pyridinyl)benzonitrile hydrochloride (460 mg, 54.5%) as white powder.

¹H NMR (200 MHz, DMSO-d₆, δ): 2.50 (2H, m), 2.70 (2H, br), 3.80 (2H, br), 6.42 (1H, m), 7.68 (2H, d, J=8.6 Hz), 7.86 (2H, d, J=8.6 Hz), 9.05 (2H, br)

Preparation 15

A mixture of 2-amino-3-iodobenzoic acid (1.12 g) and thionyl chloride (3.11 ml) was refluxed for 1 hour. The mixture was cooled, concentrated and co-evaporated with toluene twice. To 28% ammonia aqueous solution was added dropwise a solution of the residue in dichloromethane, then the resulting powder was collected, washed with water and dried in vacuo to give the 2-amino-3-iodobenzamide.

¹H NMR (DMSO-d₆, δ): 6.37 (1H, t, J=7.8 Hz), 6.58 (2H, brs), 7.30 (1H, brs), 7.59 (1H, dd, J=1.4 Hz,J=7.8 Hz), 7.90 (1H, brs).

Mass (ESI): 285.1 (M⁺+Na)

Preparation 16

The following compounds are prepared in a similar manner to that of Preparation 15.

(1) 2-Amino-3-ethylbenzamide

¹H NMR (DMSO-d₆, δ): 1.13 (3H, t, J=7.4 Hz), 2.45 (2H, q, J=7.4 Hz), 6.20-6.70 (3H, m), 6.80-7.20 (2H, m), 7.42 (1H, dd, J=1.3, 7.9 Hz), 7.71 (1H, brs)

Mass (ESI): 187.2 (M⁺+Na)

(2) 2-amino-3-bromobenzamide

Mass (ESI): 239.1 (M⁺+Na)

Preparation 17

Under a nitrogen atmosphere, a solution of 4-bromobutyryl chloride (4.9 g, 26.4 mmol) in dichloromethane (10 mL) was added dropwise to the solution of 2-aminobenzamide (3.0 g, 22 mmol) in pyridine (18 mL, 220 mmol) and dichloromethane (15 mL) at 0° C. The mixture was stirred for 1.5 hours at 0° C. The reaction mixture was poured into ice-cooled 1N hydrochloric acid, and the product was extracted with chloroform. The organic layer was washed with 1N hydrochloric acid and water and dried over sodium sulfate. The crude product was triturated with toluene to give 2-[(4-bromobutanoyl)amino]benzamide (5.11 g, 81.3%) as powder.

¹H NMR (200 MHz, CDCl₃, δ): 2.29 (2H, quint., J=6.8 Hz), 2.61 (2H, t, J=7.2 Hz), 3.52 (2H, t, J=6.4 Hz), 5.5-6.5 (2H, br), 7.09 (1H, dt, J=7.6, 1.1 Hz), 7.51 (1H, t, J=7.6 Hz), 7.53 (1H, d, J=7.6 Hz), 8.62 (1H, d, J=8.5 Hz), 11.25 (1H, s)

Mass (API-ES) 307.1, 309.1 (M⁺+Na)

Preparation 18

The following compounds are prepared in a similar manner to that of Preparation 17.

(1) 2-[(4-Bromobutanoyl)amino]-3-iodobenzamide

¹H NMR (DMSO-d₆, δ): 1.90-2.30 (2H, m), 2.43 (2H, t, J=7.4 Hz), 3.61 (2H, t, J=6.7 Hz), 7.10 (1H, t, J=7.8 Hz), 7.96 (1H, dd, J=1.3 Hz, J=7.8 Hz), 9.66 (1H, brs)

Mass (ESI): 433.0 (M⁺+Na)

(2) 3-Bromo-2-[(4-bromobutanoyl)amino]benzamide

¹H NMR (DMSO-d₆, δ): 1.80-2.10 (2H, m), 2.69 (2H, t, J=7.3 Hz), 3.51 (2H, t, J=6.3 Hz), 7.10-9.70 (6H, m)

Mass (ESI): 387.0 (M⁺+Na)

(3) 2-[(4-Bromobutanoyl)amino]-3-ethylbenzamide

¹H NMR (DMSO-d₆, δ): 0.90-3.80 (11H, m), 7.00-9.70 (6H, m)

Mass (ESI): 335.1 (M⁺+H)

(4) 2-[(4-bromobutanoyl)amino]-6-fluorobenzamide

MS (API-ES): 325.0 (M⁺+Na)

(5) 2-[(3-bromopropanoyl)amino]benzamide

MS (API-ES): 293.1 (M⁺+Na)

Preparation 19

A mixture of 2-aminobenzamide (45 mg), 4-(4-phenyl-3,6-dihydro-1(2H)-pyridinyl)pentanoic acid (85.7 mg), O-(7-azabenzotriazol-1-yl)-N,N,N′,N′-tetramethyluronium hexafluorophosphate (628 mg) and diisopropylethylamine (0.288 ml) was stirred at room temperature overnight. The mixture was diluted with water and extracted with dichloromethane three times. The combined extracts were washed with water three times, dried over magnesium sulfate and concentrated. The residue was purified by preparative thin layer chromatography using 10% methanol in dichloromethane as an eluent to give the 2-{[4-(4-phenyl-3,6-dihydro-1(2H)-pyridinyl)pentanoyl]amino}benzamide.

Mass (ESI): 388.3 (M⁺+H)

Preparation 20

Under a nitrogen atmosphere, triethylamine (0.73 mL, 5.26 mmol) was added to a solution of 2-[(4-bromobutanoyl)amino]benzamide (500 mg, 1.75 mmol) and 4-phenyl-1,2,3,6-tetrahydropyridine hydrochloride (412 mg, 2.10 mmol) in N,N-dimethylformamide (5 mL) at 0° C. The mixture was allowed to warm to room temperature and stirred for 24 hour. The reaction was quenched with water, and the product was extracted with chloroform. The organic layer was washed with water and dried over sodium sulfate. Purification over silica gel chromatography gave 2-{[4-(4-phenyl-3,6-dihydro-1(2H)-pyridinyl)butanoyl]amino}benzamide (477 mg, 74.8%) as pale-yellow powder.

¹H NMR (200 MHz, CDCl₃, δ): 2.01 (2H, quint., J=7.3 Hz), 2.41-2.56 (4H, m), 2.72 (2H, t, J=5.4 Hz), 3.76 (2H, d, J=5.7 Hz), 5.4-6.3 (2H, br), 6.05 (1H, m), 7.05 (1H, t, J=7.0 Hz), 7.21-7.37 (6H, m), 7.45-7.51 (2H, m), 8.64 (1H, d, J=8.6 Hz)

Mass (APCI): 364.20 (M⁺+H)

Preparation 21

The following compounds are prepared in a similar manner to that of Preparation 20.

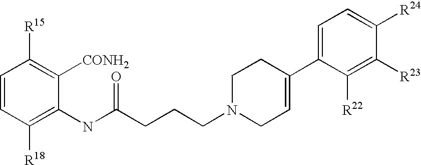

| No. | R¹⁵ | R¹⁸ | R²² | R²³ | R²⁴ | |
|---|---|---|---|---|---|---|
| (1) | H | I | H | H | H | ¹H NMR (DMSO-d₆, δ): 1.82(2H, m), 2.33(2H, t, |
| | | | | | | J=7.3Hz), 2.35-2.70(4H, m), 2.65(2H, t, J=5.4Hz), |
| | | | | | | 3.1(2H, d, J=2.8Hz), 6.15(1H, s), 6.80-7.80(9H, |
| | | | | | | m), 7.96(H, dd, J=1.4Hz, J=7.9Hz), 9.62(1H, s) |
| | | | | | | Mass(ESI): 490.2(M⁺ + Na) |
| (2) | H | Br | H | H | H | ¹H NMR(DMSO-d₆, δ): 1.70-2.00(2H, m), |
| | | | | | | 2.10-2.90(8H, m), 3.22(2H, d, J=6.1Hz), 6.16(1H, |
| | | | | | | s), 7.10-8.00(10H, m), 9.62(1H, brs) |
| | | | | | | Mass(APCI): 442.13(M⁺ + H) |
| (3) | H | Et | H | H | H | ¹H NMR(DMSO-d₆, δ): 1.10(3H, t, J=7.5Hz), |
| | | | | | | 1.60-1.90(2H, m), 2.20-2.80(10H, m), 3.09(2H, d, |
| | | | | | | J=2.6Hz), 6.16(1H, s), 7.10-7.70(10H, m), 9.38 |
| | | | | | | (1H, s) |
| | | | | | | Mass(APCI): 392.07(M⁺ + H) |
| (4) | H | H | F | H | H | Mass(APCI): 381.93(M⁺ + H) |
| (5) | H | H | H | F | H | Mass(APCI): 381.93(M⁺ + H) |
| (6) | H | H | OMe | H | H | Mass(APCI): 394.20(M⁺ + H) |
| (7) | H | H | H | OMe | H | Mass(APCI): 394.13(M⁺ + H) |
| (8) | H | H | H | H | OEt | Mass(API-ES): 408.3(M⁺ + H) |
| (9) | H | H | H | H | SMe | Mass(API-ES): 410.2(M⁺ + H) |
| (10) | H | H | H | H | OCF₃ | Mass(API-ES): 448.2(M⁺ + H) |
| (11) | H | H | H | H | Et | Mass(APCI) 390.07(M⁻ − H) |
| (12) | H | H | H | H | N(Me)₂ | Mass(APCI): 406.93(M⁺ + H) |
| (13) | H | H | H | H | t-Bt | Mass(APCI): 420.13(M⁺ + H) |
| (14) | H | H | H | H | Ph | Mass(APCI): 440.13(M⁺ + H) |
| (15) | H | H | H | H | OPh | Mass(APCI): 456.13(M⁺ + H) |
| (16) | H | H | H | H | Ac | Mass(APCI): 406.07(M⁺ + H) |
| (17) | F | H | H | H | H | Mass(API-ES): 382.4(M⁺ + H) |
| (18) | F | H | H | H | OMe | Mass(APCI): 411.80(M⁺ + H) |
| (19) | F | H | H | H | F | Mass(APCI): 399.87(M⁺ + H) |
| (20) | H | Cl | H | H | CN | Mass(API-ES): 423.3(M⁺ + H) |
| (21) | H | Cl | H | H | Ac | Mass(APCI): 440.07(M⁺ + H) |
| (22) | Cl | H | H | H | CN | Mass(API-ES): 423.3(M⁺ + H) |
| (23) | H | H | H | H | Me | ¹H NMR(200MHz, CDCl₃, δ): 2.01(2H, quint., |
| | | | | | | J=7.3Hz), 2.45-2.59(4H, m), 2.71(2H, t, J=5.6Hz), |
| | | | | | | 3.17(2H, d, J=3.2Hz), 5.4-6.4(2H, br), 6.01(1H, |
| | | | | | | m), 7.02(1H, t, J=6.5Hz), 7.11(2H, d, J=8.1Hz), |
| | | | | | | 7.25(2H, d, J=8.1Hz), 7.45-7.53(2H, m), 8.65(1H, |
| | | | | | | d, J=8.6Hz), 11.14(1H, s) |
| | | | | | | Mass(APCI): 378.20(M⁺ + H) |
| (24) | H | H | H | H | F | ¹H NMR(200MHz, CDCl₃, δ): 2.00(2H, quint., |
| | | | | | | J=7.3Hz), 2.45-2.59(6H, m), 2.71(2H, t, J=5.6Hz), |
| | | | | | | 3.16(2H, q, J=2.2Hz), 5.4-6.3(2H, br), 5.99(1H, |
| | | | | | | m), 6.97(2H, t, J=8.8Hz), 7.05(1H, t, J=7.6Hz), |
| | | | | | | 7.31(2H, dd, J=8.8, 5.4Hz), 7.45-7.52(2H, m), 8.65 |
| | | | | | | (1H, d, J=8.6Hz), 11.16(1H, s) |
| | | | | | | Mass(APCI): 381.93(M⁺ + H) |
| (25) | H | H | H | H | OMe | ¹H NMR(200MHz, CDCl₃, δ): 2.00(2H, quint., |
| | | | | | | J=7.3Hz), 2.45-2.58(6H, m), 2.70(2H, t, J=5.6Hz), |
| | | | | | | 3.16(2H, q, J=2.8Hz), 3.80(3H, s), 5.6-6.4(2H, |
| | | | | | | br), 5.96(1H, m), 8.37(2H, d, J=8.8Hz), 7.04(1H, |
| | | | | | | t, J=8.7Hz), 7.29(2H, d, J=8.8Hz), 7.44-7.52(2H, |
| | | | | | | m), 8.63(1H, dd, J=8.6, 1.2Hz), 11.15(1H, s) |
| | | | | | | Mass(APCI): 394.13(M⁺ + H) |
| (26) | H | H | H | H | CF₃ | ¹H NMR(200MHz, CDCl₃, δ): 2.01(2H, quint., |
| | | | | | | J=7.2Hz), 2.46-2.60(6H, m), 2.73(2H, t, J=5.6Hz), |
| | | | | | | 3.20(2H, q, J=2.9Hz), 5.5-6.4(2H, br), 6.14(1H, |
| | | | | | | m), 7.05(1H, t, J=7.4Hz), 7.41-7.60(6H, m), 8.65 |
| | | | | | | (1H, dd, J=8.6, 1.2Hz), 11.17(1H, s) |
| | | | | | | Mass(APCI): 432.00(M⁺ + H) |
| (27) | H | H | H | H | CN | ¹H NMR(200MHz, CDCl₃, δ): 2.01(2H, quint., |
| | | | | | | J=7.2Hz), 2.45-2.61(6H, m), 2.73(2H, t, J=5.6Hz), |
| | | | | | | 3.63(2H, q, J=6.1Hz), 5.4-6.3(2H, br), 6.28(1H, |
| | | | | | | m), 7.05(1H, t, J=7.7Hz), 7.40-7.61(4H, m), 7.58 |
| | | | | | | (2H, d, J=8.5Hz), 8.65(1H, d, J=8.6Hz), 11.17(1H, |
| | | | | | | s) |
| | | | | | | Mass(APCI): 389.00(M⁺ + H) |
| (28) | H | H | H | H | CH₂OH | ¹H NMR(DMSO-d₆, δ): 1.75-1.95(2H, m), 2.3-2.7 |
| | | | | | | (8H, m), 3.07(2H, d, J=2.7Hz), 4.47(2H, d, J=5.6 |
| | | | | | | Hz), 5.12(1H, t, J=5.6Hz), 6.11(1H, s), 7.08(1H, |
| | | | | | | dt, J=7.4, 1.1Hz), 7.25(2H, d, J=8.3Hz), 7.36(2H, |
| | | | | | | d, J=8.3Hz), 7.46(1H, dt, J=7.4, 1.4Hz), 7.69(1H, |
| | | | | | | brs), 7.77(1H, dd, J=7.9, 1.4Hz), 8.24(1H, brs), |
| | | | | | | 8.47(1H, brs), 11.67(1H, s) |
| | | | | | | Mass: 394.1(M⁺) |
| (29) | H | Cl | H | H | OMe | ¹H NMR(DMSO-d₆, δ): 1.7-1.95(2H, m), 2.3-2.75 |
| | | | | | | (6H, m), 3.09(2H, s), 3.74(3H, s), 6.03(1H, s), 6.88 |
| | | | | | | (2H, d, J=8.9Hz), 7.25-7.65(9H, m), 9.65(1H, s) |
| | | | | | | Mass: 428.1(M⁺ + H) |
| (30) | H | Cl | H | H | H | ¹H NMR(DMSO-d₆, δ): 1.7-1.95(2H, m), 2.25-2.7 |
| | | | | | | (6H, m), 3.08(2H, d, J=2.5Hz), 6.15(1H, s), 7.2-7.7 |
| | | | | | | (10H, m) |
| | | | | | | Mass: 398.3(M⁺ + H) |
| (31) | H | Cl | H | H | CF₃ | ¹H NMR(DMSO-d₆, δ): 1.7-1.9(2H, m), 2.25-2.75 |
| | | | | | | (6H, m), 3.12(2H, m), 6.33(1H, s), 7.25-7.70(9H, |
| | | | | | | m), 9.61(1H, s) |
| | | | | | | Mass: 466.0(M⁺) |
| (32) | H | Cl | H | H | CH₂OH | ¹H NMR(DMSO-d₆, δ): 1.7-1.9(2H, m), 2.25-2.75 |
| | | | | | | (6H, m), 3.08(2H, m), 4.47(2H, d, J=5.8Hz), 5.12 |
| | | | | | | (1H, t, J=5.8Hz), 6.13(1H, s), 7.2-7.6(9H, m), 9.61 |
| | | | | | | (1H, s) |
| | | | | | | Mass: 428.1(M⁺ + H) |

Preparation 22

The following compounds are prepared in a similar manner to that of Preparation 20.

(1) 2-({4-[4-(4-methylphenyl)-1-piperidyl]butanoyl}amino)benzamide

Mass (APCI): 379.93 (M⁺+H)

(2) 2-{[4-(4-phenyl-1-piperazinyl)butanoyl]amino}benzamide

Mass (APCI): 367.07 (M⁺+H)

Preparation 23-(1)

Palladium hydroxide on carbon (10%, 51.4 mg, 0.0366 mmol) was added to a solution of 2-({4-[4-[4-(methylthio)phenyl]-3,6-dihydro-1(2H)-pyridinyl]butanoyl}amino)benzamide (150 mg, 0.366 mmol) in a mixed solvent of methanol (2 mL) and ethyl acetate (2 mL). Purged by hydrogen (1 atm), the mixture was stirred at room temperature for 2 days. Purification over silica gel chromatography gave 2-[(4-{4-[4-(methylthio)phenyl]1-piperidyl}butanoyl)amino]benzamide (44 mg, 29.2%) as product.

Mass (APCI): 412.27 (M⁺⁺H)

Preparation 23-(2)

Palladium on carbon (10%, 37.5 mg, 0.0352 mmol) was added to a solution of 2-{[4-(4-phenyl-3,6-dihydro-1(2H)-pyridinyl)butanoyl]amino}benzamide (128 mg, 0.352 mmol) in a mixed solvent of methanol (2 mL) and ethyl acetate (3 mL). Purged by hydrogen (1 atm), the mixture was stirred at room temperature for 10 hour. Purification over silica gel chromatography gave 2-{[4-(4-phenyl-1-piperidyl)butanoyl]amino}benzamide (91 mg, 70.7%) as product.

Mass (APCI): 366.13 (M⁺+)

Preparation 24

The following compounds are prepared in a similar manner to that of Preparation 23-(2).

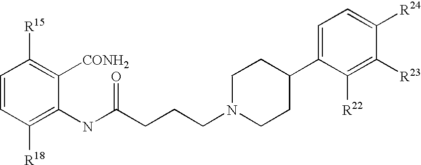

| | R¹⁵ | R¹⁸ | R²² | R²³ | R²⁴ | |
|---|---|---|---|---|---|---|
| (1) | H | H | H | H | OEt | Mass(API-ES): 410.4(M⁺ + H) |
| (2) | H | H | H | H | OCF₃ | Mass(API-ES): 450.3(M⁺ + H) |
| (3) | H | H | H | H | Et | Mass(APCI): 393.87(M⁺ + H) |
| (4) | H | H | H | H | N(Me)2 | Mass(APCI): 409.27(M⁺ + H) |
| (5) | H | H | H | H | Ph | Mass(APCI): 442.27(M⁺ + H) |
| (6) | H | H | H | H | OPh | Mass(APCI): 458.27(M⁺ + H) |
| (7) | H | H | H | H | Ac | Mass(APCI): 408.13(M⁺ + H) |
| (8) | H | H | H | H | OMe | Mass(APCI): 414.00(M⁺ + H) |
| (9) | H | H | H | H | F | Mass(APCI): 401.93(M⁺ + H) |
| (10) | H | H | H | H | CF₃ | Mass(APCI): 434.07(M⁺ + H) |
| (11) | H | H | H | H | F | ¹H NMR(DMSO-d₆, δ): 1.4-2.1(8H, m), 2.25-2.6 |
| | | | | | | (5H, m), 2.91(2H, t, J=11.6Hz), 6.95-7.20(5H, m), |
| | | | | | | 7.4-7.5(1H, m), 7.69(1H, brs), 7.80(1H, d, J=7.3 |
| | | | | | | Hz), 8.24(1H, brs), 8.53(1H, d, J=8.4Hz), 11.75 |
| | | | | | | (1H, s) |
| (12) | H | H | H | H | OMe | ¹H NMR(DMSO-d₆, δ): 1.4-2.05(8H, m), 2.25-2.45 |
| | | | | | | (3H, m), 2.85-3.0(2H, m), 3.70(3H, s), 6.79(2H, d, |
| | | | | | | J=8.7Hz), 7.02(2H, d, J=8.7Hz), 7.11(1H, dt, J=7.9, |
| | | | | | | 1.1Hz), 7.48(1H, dt, J=7.5, 1.1Hz), 7.69(1H, brs), |
| | | | | | | 7.81(1H, dd, J=7.9, 1.4Hz), 8.24(1H, brs), 8.53 |
| | | | | | | (1H, dd, J=8.3, 0.9Hz), 11.75(1H, s) |

Preparation 25

The following compounds are prepared in a similar manner to that of Preparation 20.

(1) 2-{[3-(4-phenyl-3,6-dihydro-1(2H)-pyridinyl)propanoyl]amino}benzamide

MS (APCI). 350.00 (M⁺+H)

(2) 2-{[5-(4-phenyl-3,6-dihydro-1(2H)-pyridinyl)pentanoyl]amino}benzamide

¹H NMR (200 MHz, CDCl₃, δ): 1.6-1.9 (4H, m), 2.4-2.6 (6H, m), 2.71 (2H, t, J=5.4 Hz), 3.16 (2H, q, J=2.9 Hz), 5.4-6.5 (2H, br), 6.05 (1H, m), 7.07 (1H, t, J=7.5 Hz), 7.2-7.5 (5H, m), 7.5-7.6 (2H, m), 8.67 (1H, d, J=8.6 Hz), 11.17 (1H, br s)

(3) 2-{[3-(4-benzyl-1-piperidyl)propanoyl]amino}benzamide

MS (APCI): 366.07 (M⁺+H)

(4) 2-{[3-(4-benzyl-1-piperazinyl)propanoyl]amino}benzamide

MS (APCI): 367.00 (M⁺+H)

Preparation 26

The following compounds are prepared in a similar manner to that of Preparation 20.

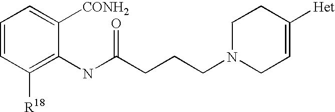

| No. | R¹⁸ | Het | |
|---|---|---|---|
| (1) | H | 1,3-thiazol-2-yl | Mass(APCI): 370.73(M⁺ + H) |
| (2) | H | 1-methyl-1H- | Mass(APCI): 367.93(M⁺ + H) |
| | | imidazol-2-yl | |
| (3) | H | 1-methyl-1H- | Mass(APCI): 368.00(M⁺ + H) |
| | | pyrazol-5-yl | |
| (4) | H | 2-thienyl | Mass(APCI): 369.80(M⁺ + H) |
| (5) | Cl | 2-thienyl | Mass(APCI): 403.87(M⁺ + H) |
| (6) | H | 3-thienyl | Mass(APCI): 369.93(M⁺ + H) |
| (7) | Cl | 3-thienyl | Mass(APCI): 403.93(M⁺ + H) |
| (8) | H | 4-methyl-2-thienyl | Mass(APCI): 384.00(M⁺ + H) |
| (9) | H | 5-acetyl-2-thienyl | Mass(APCI): 312.07(M⁺ + H) |
| (10) | H | 5-chloro-2-thienyl | Mass(APCI): 403.93(M⁺ + H) |
| (11) | H | 5-cyano-2-thienyl | Mass(APCI): 395.13(M⁺ + H) |
| (12) | H | 5-methyl-2-thienyl | Mass(APCI): 384.3(M⁺ + H) |
| (13) | H | 2-pyridinyl | Mass(APCI): 364.93(M⁺ + H) |
| (14) | H | 3-pyidinyl | Mass(APCI): 365.00(M⁺ + H) |
| (15) | Cl | 4-pyridinyl | ¹H NMR(200MHz, DMSO-d₆, δ): 1.7-1.9(2H, m), |
| | | | 2.25-2.55(6H, m), 2.6-2.7(2H, m), 3.12(2H, d, J=2.5Hz), |
| | | | 6.49(1H, s), 7.25-7.6(5H, m), 8.50(2H, dd, J=4.6, 1.6Hz), |
| | | | 9.61(1H, s) |
| | | | Mass: 399.1(M⁺ + H) |
| (16) | H | 4-pyridinyl | Mass(APCI): 364.93(M⁺ + H) |

Preparation 27

The following compounds are prepared in a similar manner to that of Preparation 23-(2).

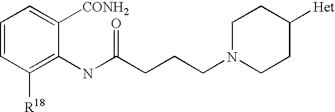

| No. | R¹⁸ | Het | |
|---|---|---|---|
| (1) | H | 1-methyl-1H- | Mass(API-ES): 370.4(M⁺ + H) |
| | | pyrazol-5-yl | |
| (2) | H | 2-thienyl | Mass(API-ES): 372.3(M⁺ + H) |
| (3) | H | 3-thienyl | MS(APCI): 372.07(M⁺ + H) |
| (4) | H | 4-methyl-2-thienyl | Mass(APCI): 386.13(M⁺ + H) |
| (5) | H | 5-methyl-2-thienyl | Mass(APCI): 386.07(M⁺ + H) |
| (6) | H | 4-pyridinyl | Mass(APCI): 365.00(M⁺ + H) |

Preparation 28

The following compounds are prepared in a similar manner to that of Preparation 20.

(1) 2-({4-[4-(4-Chlorophenyl)-3-oxo-1-piperazinyl]butanoyl}amino)benzamide

¹H NMR (DMSO-d₆, δ): 1.70-2.00 (2H, m), 2.20-2.70 (6H, m), 2.76 (2H, t, J=5.3 Hz), 3.60 (2H, t, J=5.3 Hz), 6.30-8.70 (10H, m), 11.71 (1H, brs)

Mass (ESI): 437.3 (M⁺+Na)

(2) 2-{[4-(3-phenyl-1-pyrrolidinyl)butanoyl]amino}benzamide

Mass (APCI): 352.27 (M⁺+H)

(3) 2-{[4-(4-phenyl-1H-imidazol-1-yl)butanoyl]amino}benzamide

¹H NMR (200 MHz, CDCl₃, δ): 2.25 (2H, quint., J=6.8 Hz), 2.44 (2H, t, J=6.1 Hz), 4.08 (2H, t, J=6.8 Hz), 6.0-6.9 (2H, br), 7.05 (1H, t, J=7.6 Hz), 7.1-7.7 (7H, m), 7.75 (2H, d, J=8.1 Hz), 8.62 (1H, d, J=8.4 Hz), 11.40 (1H, br s)

(4) 2-{[4-(1,4,5,6-tetrahydrobenzo[f]isoquinolin-3(2H)-yl)butanoyl]amino}benzamide

Mass (APCI): 389.73 (M⁺+H)

(5) 2-{[4-(spiro[1H-indene-1,4′-piperidin-1′-yl])butanoyl]amino}benzamide Mass

(APCI): 390.13 (M⁺+H)

(6) 2-{[4-(2,3-dihydrospiro[1H-indene-1,4′-piperidin-1′-yl])butanoyl]amino}-benzamide

Mass (APCI): 392.20 (M⁺+H)

Preparation 29

2-{[4-(4-phenyl-2,3,6,7-tetrahydro-1H-azepin-1-yl)butanoyl]amino}benzamide (142 mg, 25.1%) and 2-{[4-(5-phenyl-2,3,4,7-tetrahydro-1H-azepin-1-yl)butanoyl]-amino}benzamide (121 mg, 21.4%) were synthesized from 2-[(4-bromobutanoyl)-amino]benzamide (427 mg, 1.50 mmol) and a mixture of 5-phenyl-2,3,4,7-tetrahydro-1H-azepine hydrochloride and 4-phenyl-2,3,6,7-tetrahydro-1H-azepine hydrochloride (345 mg, 1.65 mmol) by a similar procedure to the Preparation 20.

2-{[4-(4-phenyl-2,3,6,7-tetrahydro-1H-azepin-1-yl)butanoyl]amino}benzamide

Mass (APCI): 378.20 (M⁺+H)

2-{[4-(5-phenyl-2,3,4,7-tetrahydro-1H-azepin-1-yl)butanoyl]amino}benzamide

Mass (APCI): 378.20 (M⁺+H)

Preparation 30

The following compounds are prepared in a similar manner to that of Preparation 23-(2).

(1) 2-{[4-(4Phenylhexahydro-1H-azepin-1-yl)butanoyl]amino}benzamide

Mass (APCI): 380.27 (M⁺+H)

(2) 2-{[4-(cis-1,4,4a,5,6,10b-hexahydrobenz[f]isoquinolin-3(2H)-yl)butanoyl]-amino}benzamide

Mass (API-ES): 392.4 (M⁺+H)

Preparation 31

Dimethylformamide (1.25 mL, 16.2 mmol) and oxaryl chloride (1.41 mL, 16.2 mmol) were added to a solution of 6-[(benzyloxy)carbonylamino]hexanoic acid (3.9 g, 14.7 mmol) in dichloromethane (5 mL) at 5° C. The prepared 6-{[(benzyloxy)carbonyl]amino}hexanoyl chloride was added to a solution of 2-aminobenzamide and diisopropylethylamine (2.8 mL, 1.1 eq) in dichrolomethane (5 mL) at 5° C. The mixture was stirred at room temperature for 2 hours. The mixture was extracted with AcOEt, and washed with saturated sodium hydrogen carbonate aqueous solution and brine. The organic layer was dried over MgSO₄ and the solvent was removed in vacuo to give 2-{[5-[(benzyloxy)carbonylamino]hexanoyl]amino}benzamide (2.8 g, 7.3 mmol, 50%) as yellow oil.

Mass: 384 (M⁺+1)

### EXAMPLE 1

1,2,3,6-Tetrahydro-4-phenylpyridine (54.8 g, 280 mmol) was added to the 10% aqueous acetonitrile solution of

8-chloro-1-hydroxy-2,3-dihydropyrrolo[2,1-b]quinazoline-9(1H)-one (26.5 g, 112 mmmol), then sodium cyanoborohydride (10.5 g, 168 mmol) and acetic acid (8.9 mL, 157 mmol) was added to the reaction mixture. The mixture was stirred at room temperature over night. Saturated sodium hydrogen carbonate aqueous solution was added to the reaction mixture. The precipitate was corrected with filtration and purified by silica gel chromatography eluting with chloroform and methanol (100:1-100:2). The resulting solid was recrystallized from 10% aqueous acetonitrile to give 5-chloro-2-[3-(4-phenyl-3,6-dihydro-1(2H)-pyridinyl)propyl]-4(3H)-quinazolinone (17 g, 44 mmol, 40%) as colorless fine needle.

¹H NMR (300 MHz, CDCl₃, δ): 2.05 (2H, quint, J=6.2 Hz), 2.66 (2H, t, J=6.2 Hz), 2.80-2.92 (6H, m), 3.31 (1H, m), 6.118 (1H, s), 7.32-7.47 (6H, m), and 7.55 (2H, m).

Mass (m/z): 380 (M⁺+1)

### EXAMPLE 2

4-Phenylpiperidine hydrochloride (334 mg, 1.69 mmol) was added to the 10% aqueous acetonitrile solution of 8-chloro-1-hydroxy-2,3-dihydropyrrolo[2,1-b]quinazoline-9(1H)-one (200 mg, 0.85 mmol), then sodium cyanoborohydride (133 mg, 2.11 mmol) and acetic acid (0.1 mL, 1.69 mmol) were added to the reaction mixture. The mixture was stirred at room temperature over night. The reaction mixture was extracted with ethyl acetate and washed with saturated sodium hydrogen carbonate aqueous solution and brine. The organic layer was dried over MgSO₄ and the solvent was removed in vacuo. The residue was purified by silica gel chromatography eluting with chloroform and methanol (100:5) to give 5-chloro-2-[3-(4-phenyl-1-piperidyl)propyl]-4(3H)-quinazolinone (96.6 mg, 0.25 mmol, 30%) as colorless solid.

¹H NMR (300 MHz, CDCl₃, δ): 1.88 (2H, m), 2.00 (2H, m), 2.25 (2H, m), 2.28 (5H, m), 2.60 (2H, m), 2.86 (2H, m), 3.19 (2H, m), 7.33-7.41 (6H, m), and 7.53 (2H, m).

Mass (m/z): 382 (M⁺+1)

### EXAMPLE 3

4-Cyano-4-phenylpiperidine hydrochloride (452 mg, 2.03 mmol) was added to the 10% aqueous acetonitrile solution of 8-chloro-1-hydroxy-2,3-dihydropyrrolo[2,1-b]quinazoline-9(1H)-one (160 mg, 0.676 mmol), then sodium cyanoborohydride (42.4 mg, 0.676 mmol) and acetic acid (46 mL) were added to the reaction mixture. The reaction mixture was stirred at room temperature for 4 hours. The mixture was extracted with ethyl acetate and washed with saturated sodium hydrogen carbonate aqueous solution and brine. The organic layer was dried over magnesium sulfate and the solvent was removed in vacuo. The residue was purified by preparative TLC, and recrystallized from methanol to give 5-chloro-2-[3-(4cyano4-phenyl-1-piperidyl)propyl]4(3H)-quinazolinone (22 mg, 0.055 mmol, 8%) as colorless powder.

¹H NMR (300 MHz, CDCl₃, δ): 2.01(2H, quint, J=5.5 Hz), 2.12 (2H, m), 2.73-2.67 (6H, m), 2.92 (2H, m), 3.22 (2H, m), 7.43-7.48(4H, m), 7.54(2H, m) and 7.77 (2H, m)

Mass (m/z): 407 (M⁺+1)

### EXAMPLE 4

4-Hydroxy-4-phenylpiperidine hydrochloride(592 mg, 3.34 mmol) was added to the 10% aqueous acetonitrile solution of 8-chloro-1-hydroxy-2,3-dihydropyrrolo[2,1-b]quinazoline-9(1H)-one (395 mg, 3.34 mmol), then sodium cyanoborohydride (157 mg, 2.5 mmol) and acetic acid (0.15 mL) were added to the reaction mixture. The reaction mixture was stirred at room temperature for 4 hours. The mixture was extracted with ethyl acetate and washed with saturated sodium hydrogen carbonate aqueous solution and brine. The organic layer was dried over magnesium sulfate and the solvent was removed in vacuo. The residue was purified by silica gel chromatography eluting with chloroform and methanol (100:5-50:50), and the obtained colorless solid was washed with ether to give 5-chloro-2-[3-(4-hydroxy-4-phenyl-1-piperidyl)propyl]-4(3H)-quinazolinone (190 mg, 0.48 mmol, 29%) as colorless powder.

¹H NMR (300 MHz, CDCl₃, δ): 1.82(2H, d, J=5.5 Hz), 2.01 (2H, m), 2.65-2.77 (6H, m), 2.90 (2H, m), 3.00 (2H, d, J=9.5 Hz), 7.30 (1H, dm, J=8.7 Hz), 7.43-7.48(3H, m), 7.53(2H, m) and 7.71 (2H, dm, J=7.3 Hz)

Mass (m/z): 398 (M⁺+1)

### EXAMPLE 5

4-Amino-4-phenylpiperidine (150 mg, 0.85 mmol) was added to 10% aqueous acetonitrile solution of 8-chloro-1-hydroxy-2,3-dihydropyrrolo[2,1-b]quinazoline-9(1H)-one (181 mg, 0.77 mmol). NaBH₃CN (64.1 mg, 1.02 mmol) and AcOH (0.146 mL, 2.55 mmol) were added to the mixture, and the mixture was stirred at room temperature for 4 hours. The reaction mixture was extracted with AcOEt, washed with saturated sodium hydrogen carbonate aqueous solution. Residual solid was purified by preparative TLC (chloroform/methanol 75:25) to give 2-[3-(4-amino-4-phenyl-1-piperidyl)propyl]-5-chloro-4(3H)-quinazolinone (3.5 mg, 0.008 mmol, 1%) as colorles powder.

¹H NMR (300 MHz, CDCl₃, δ): 1.86 (2H, m), 1.97 (2H, m), 2.58 (4H, m), 2.74 (4H, m), 2.86 (2H, m), 7.25 (1H, m), 7.38 (3H, m), 7.52 (2H, m), and 7.63 (2H, d, J=7.8 Hz).

Mass (m/z): 397 (M⁺+1)

### EXAMPLE 6

To a solution of 2-[4-(1-phenyl-4-piperidyl)-butanoylamino]benzamide in 1,4-dioxane (6 mL) was added 1N aqueous NaOH (6 mL). The mixture was stirred for 1 hour at room temperature, then H₂O was added and neutralized with 1N aqueous HCl. A white precipitate was filtered ,washed with Et₂O and dried at 40° C. to give 2-[3-(1-phenyl-4-piperidyl)propyl]-4(3H)-quinazolinone (75 mg, 0.21 mmol, 79%) as a pale yellow powder.

¹H NMR (300 MHz, CDCl₃, δ): 1.29-1.55 (5H, m), 1.84 (2H, d, J=10.6 Hz), 1.89-2.04 (2H, m), 2.68 (2H, t, J=10.0 Hz), 2.80 (2H, t, J=7.7 Hz), 3.66 (2H, d, 12.8 Hz), 6.82 (1H, t, J=7.0 Hz), 6.93 (2H, d, J=6.9 Hz), 7.15-7.30 (1H, m), 7.47 (1H, t, J=8.1 Hz), 7.66-7.85 (2H, m), 8.29 (1H, d, J=8.1 Hz), 11.36 (1H, s)

Mass: 348 (M⁺)

### EXAMPLE 7

A mixture of 3-nitroisatoic anhydride (0.11 g) and 4-(4-phenyl-3,6-dihydro-1(2H)-pyridinyl)butanimidamide (154 mg) in pyridine was refluxed for 24 hours. The mixture was diluted with water and extracted with dichloromethane three times. The combined extracts were dried over magnesium sulfate, concentrated and co-evaporated with toluene twice. The residue was purified by preparative thin layer chromatography on silica gel using 10% methanol in dichloromethane as an eluent to give 8-nitro-2-[3-(4-phenyl-3,6-dihydro-1(2H)-pyridinyl)propyl]-4(3H)-quinazolinone as a yellow powder.

¹H NMR (DMSO-d₆, δ): 1.80-2.10 (2H, m), 2.40-3.30 (10H, m), 6.02 (1H, s), 7.10-8.60 (8H, m).

Mass (ESI): 391.2 (M⁺+H)

### EXAMPLE 8

Under a nitrogen atmosphere, triethylamine (1.39 mL, 10.0 mmol) was added to a solution of 2-[(4-bromobutanoyl)amino]benzamide (285 mg, 1.00 mmol) and 4-phenyl-4-piperidinol (266 mg, 1.50 mmol) in N,N-dimethylformamide (3 mL) at 0° C. The mixture was allowed to warm to room temperature and stirred for 13 hours. The reaction was quenched with water, and the product was extracted with chloroform. The organic layer was washed with water and dried over sodium sulfate. The crude 2-{[4-(4-hydroxy-4-phenyl-1-piperidyl)butanoyl]amino}benzamide was dissolved in dioxane (3 mL). An aqueous solution of sodium hydroxide (1M, 3 mL) was added to the solution at room temperature, and the mixture was stirred at that temperature for 3 hour. The organic materials were extracted with chloroform, and the organic layer was washed with water and dried over sodium sulfate. Recrystalization of the crude product from chloroform-methanol gave 2-[3-(4-hydroxy-4-phenyl-1-piperidyl)propyl]-4(3H)-quinazolinone (223 mg, 61.4%).

¹H NMR (200 MHz, CDCl₃, δ): 1.7-1.9 (4H, m), 2.00 (2H, quint., J=5.4 Hz), 2.6-2.8 (5H, m), 2.9-3.1 (4H, m), 7.29 (2H, t, J=6.2 Hz), 7.42 (3H, t, J=7.4 Hz), 7.64 (1H, t, J=6.8 Hz), 7.73 (2H, d, J=8.1 Hz), 8.28 (1H, d, J=7.9 Hz)

Mass (APCI): 364.00 (M⁺+H)

### EXAMPLE 9

2-{[4-(4-Phenyl-3,6-dihydro-1(2H)-pyridinyl)butanoyl]amino}benzamide (475 mg, 1.31 mmol) was dissolved in dioxane (5 mL). An aqueous solution of sodium hydroxide (1M, 3.92 mL) was added to the solution at room temperature, and the mixture was stirred at that temperature for 15 hours. The organic materials were extracted with chloroform, and the organic layer was washed with water and dried over sodium sulfate. Recrystalization of the crude product from chloroform-methanol gave 2-{3-[4-phenyl-3,6-dihydro-1(2M-pyridinyl]propyl}-4(3H)-quinazolinone (329 mg, 72.9%).

¹H NMR (200 MHz, CDCl₃, δ): 2.05 (2H, quint., J=6.0 Hz), 2.66 (2H, t, J=6.0 Hz), 2.81-2.94 (4H, m), 3.31 (2H, d, J=3.2 Hz), 6.12 (1H, t, J=2.9 Hz), 7.21-7.49 (7H, m), 7.61-7.72 (2H, m), 8.23 (1H, d, J=6.6 Hz)

Mass (APCI): 346.20 (M⁺+H)

### EXAMPLE 10

The following compounds are prepared in a similar manner to that of Example 9. If necessary, the starting compounds of them were prepared in similar manners of Preparation 17, Preparation 20 and preparation 23-(2)

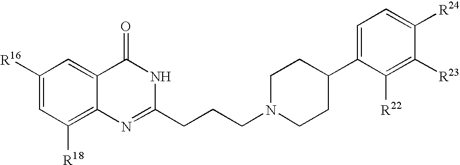

| No. | R¹⁶ | R¹⁸ | R²² | R²³ | R²⁴ | |
|---|---|---|---|---|---|---|
| (1) | H | H | H | H | OEt | ¹H NMR(200MHz, CDCl₃, δ): 1.41(3H, t, J=7.0Hz), |
| | | | | | | 1.8-2.1(4H, m), 2.1-2.4(4H, m), 2.4-2.6(3H, m), 2.9-3.0 |
| | | | | | | (2H, m), 3.19(2H, brd, J=7.7Hz), 4.03(2H, q, J=7.0 |
| | | | | | | Hz), 6.89(2H, d, J=8.7Hz), 7.2-7.5(4H, m), 7.5-7.8(2H, |
| | | | | | | m), 8.29(1H, d, J=8.0Hz) |
| | | | | | | Mass(API-ES): 392.4(M⁺ + H) |
| (2) | H | H | H | H | SMe | ¹H NMR(200MHz, CDCl₃, δ): 1.85(2H, brd, J=8.7Hz), |
| | | | | | | 1.96(2H, quint., J=5.4Hz), 2.1-2.4(4H, m), 2.48(3H, s), |
| | | | | | | 2.5-2.6(3H, m), 2.9-3.0(2H, m), 3.20(2H, brd, J=6.8 |
| | | | | | | Hz), 7.26(2H, d, J=8.4Hz), 7.36(2H, d, J=8.5Hz), 7.39 |
| | | | | | | (1H, t, J=8.2Hz), 7.6-7.8(2H, m), 8.29(1H, d, J=8.0Hz). |
| | | | | | | MS(APCI): 394.13(M⁺ + H) |
| (3) | H | H | H | H | OCF₃ | ¹H NMR(200MHz CDCl₃, δ): 1.86(2H, brd, J=9.9Hz), |
| | | | | | | 1.99(2H, quint., J=5.6Hz), 2.2-2.4(4H, m), 2.5-2.7(3H, |
| | | | | | | m), 2.9-3.0(2H, m), 3.22(2H, brd, J=9.0Hz), 7.20(2H, |
| | | | | | | d, J=7.9Hz), 7.4-7.5(3H, m), 7.63(1H, d, J=6.8Hz), |
| | | | | | | 7.68(1H, t, J=6.8Hz), 8.29(1H, d, J=7.9Hz), 14.10(1H, |
| | | | | | | br) |
| | | | | | | Mass(APCI): 432.07(M⁺ + H) |
| (4) | H | H | H | H | Et | ¹H NMR(200MHz, CDCl₃, δ): 1.24(3H, t, J=7.6Hz), |
| | | | | | | 1.8-2.1(4H, m), 2.2-2.4(4H, m), 2.4-2.7(5H, m), 2.9-3.0 |
| | | | | | | (2H, m), 3.1-3.3(2H, m), 7.19(2H, d, J=8.2Hz), 7.34 |
| | | | | | | (2H, d, J=8.1Hz), 7.42(1H, t), 7.6-7.8(2H, m), 8.2-8.4 |
| | | | | | | (1H, m) |
| | | | | | | Mass(API): 376.4(M⁺ + H) |
| (5) | H | H | H | H | N(Me)₂ | ¹H NMR(200MHZ, CDCl₃, δ): 1.7-2.1(4H, m), 2.1-2.3 |
| | | | | | | (4H, m), 2.3-2.6(3H, m), 2.9-3.0(8H, m), 3.18(2H, brd, |
| | | | | | | J=5.9Hz), 7.77(2H, d, J=8.8Hz), 7.30(2H, d, J=8.7Hz), |
| | | | | | | 7.67(1H, t), 7.6-7.7(2H, m), 8.30(1H, d, J=6.9Hz) |
| | | | | | | Mass(APCI): 391.13(M⁺ + H) |
| (6) | H | H | H | H | Ph | ¹H NMR(200MHz, DMSO-d₆, δ): 1.4-1.7(4H, m), |
| | | | | | | 1.8-2.0(4H, m), 2.3-2.5(3H, m), 2.68(2H, t, J=6.9Hz), |
| | | | | | | 2.97(2H, brd, J=10.9Hz), 7.20(2H, d, J=8.2Hz), |
| | | | | | | 7.3-7.7(9H, m), 7.77(1H, t, J=6.9Hz), 8.12(1H, d, J=7.9 |
| | | | | | | Hz), 12.43(1H, brs) |
| | | | | | | Mass(APCI): 424.20(M⁺ + H) |
| (7) | H | H | H | H | OPh | ¹H NMR(200MHz, DMSO-d₆, δ): 1.3-1.7(4H, m), |
| | | | | | | 1.8-2.0(4H, m), 2.3-2.5(3H, m), 2.67(2H, t, J=7.1Hz), |
| | | | | | | 2.95(2H, brd, J=11.0Hz), 6.90(2H, d, J=8.6Hz), 6.97 |
| | | | | | | (2H, d, J=7.5Hz), 7.1-7.2(3H, m), 7.3-7.5(3H, m), 7.59 |
| | | | | | | (1H, d), 7.76(1H, t, J=6.8Hz), 8.10(1H, d, J=8.0Hz), |
| | | | | | | 12.43(1H, brs) |
| | | | | | | Mass(API): 440.4(M⁺ + H) |
| (8) | H | H | H | H | Ac | ¹H NMR(200MHz, DMSO-d₆, δ): 1.4-1.7(4H, m), |
| | | | | | | 1.8-2.1(4H, m); 2.3-2.5(3H, m), 2.55(3H, s), 2.67(2H, t, |
| | | | | | | J=6.9Hz), 2.96(2H, brd, J=11.0Hz), 7.26(2H, d, J=8.3 |
| | | | | | | Hz), 7.46(1H, t, J=6.9Hz), 7.59(1H, d, J=7.6Hz), 7.76 |
| | | | | | | (1H, t, J=7.1Hz), 7.86(2H, d, J=8.3Hz), 8.11(1H, d, |
| | | | | | | J=7.9Hz), 12.42(1H, brs) |
| | | | | | | Mass(APCI): 390.07(M⁺ + H) |
| (9) | H | H | H | H | H | ¹H NMR(200MHz, CDCl₃, δ): 1.88(2H, d, J=9.5Hz), |
| | | | | | | 1.99(2H, quint., J=5.5Hz), 2.1-2.5(4H, m), 2.5-2.7(3H, |
| | | | | | | m), 2.9-3.0(2H, m), 3.21(2H, brd, J=7.9Hz), 7.1-7.5 |
| | | | | | | (6H, m), 7.63(1H, d, J=6.9Hz), 7.71(1H, t, J=6.8Hz), |
| | | | | | | 8.30(1H, d, J=7.9Hz) |
| | | | | | | Mass(APCI): 348.20(M⁺ + H) |
| (10) | H | H | H | H | Me | ¹H NMR(200MHz, CDCl₃, δ): 1.86(2H, brd, J=7.8Hz), |
| | | | | | | 1.94(2H, quint., J=5.9Hz), 2.1-2.4(4H, m), 2.34(3H, s), |
| | | | | | | 2.5-2.7(3H, m), 2.9-3.0(2H, m), 3.20(2H, brd, J=6.5 |
| | | | | | | Hz), 7,16(2H, d, J=7.9Hz), 7.31(2H, d, J=8.1Hz), 7.42 |
| | | | | | | (1H, t, J=8.1Hz), 7.6-7.7(2H, m), 8.2-8.3(1H, m) |
| | | | | | | Mass(API): 362.4(M⁺ + H) |
| (11) | H | H | H | H | CF₃ | ¹H NMR(200MHz, CDCl₃, δ): 1.87(2H, brd, J=11.1 |
| | | | | | | Hz), 1.93(2H, quint., J=5.7Hz), 2.1-2.5(4H, m), 2.5-2.8 |
| | | | | | | (3H, m), 2.9-3.0(2H, m), 3.23(2H, brd, J=10.4Hz), 7.43 |
| | | | | | | (1H, t, J=8.0Hz), 7.5-7.8(6H, m), 8.2-8.3(1H, m), 14.05 |
| | | | | | | (1H, br) |
| | | | | | | Mass(APCI): 416.00(M⁺ + H) |
| (12) | H | H | H | H | F | ¹H NMR(200MHz, DMSO-d₆, δ): 1.3-1.7(4H, m), |
| | | | | | | 1.8-2.1(4H, m), 2.3-2.4(3H, m), 2.67(2H, t, J=7.1Hz), |
| | | | | | | 2.94(2H, d, J=11.2Hz), 7.0-7.2(4H, m), 7.46(1H, t, |
| | | | | | | J=8.0Hz), 7.58(1H, d, J=7.5Hz), 7.7-7.8(1H, m), 8.11 |
| | | | | | | (1H, dd, J=7.9, 1.2Hz) |
| | | | | | | Mass: 365.9(M⁺) |
| (13) | H | H | H | H | OMe | ¹H NMR(200MHz, DMSO-d₆, δ): 1.3-1.7(4H, m), |
| | | | | | | 1.8-2.1(4H, m), 2.25-2.45(3H, m), 2.66(2H, t, J=7.1 |
| | | | | | | Hz), 2.93(2H, d, J=11.2Hz), 3.71(3H, s), 6.81(2H, d, |
| | | | | | | J=8.7Hz), 7.02(2H, d, J=8.7Hz), 7.45(1H, t, J=8.0Hz), |
| | | | | | | 7.58(1H, d, J=7.5Hz), 7.7-7.8(1H, m), 8.10(1H, dd, |
| | | | | | | J=7.9, 1.2Hz) |
| | | | | | | Mass: 377.8(M⁺) |
| (14) | H | Cl | H | H | F | ¹H NMR(200MHz, DMSO-d₆, δ): 1.3-1.7(4H, m), |
| | | | | | | 1.8-2.0(3H, m), 2.3-2.6(4H, m), 2.6-2.8(2H, m), 2.8-3.0 |
| | | | | | | (2H, m), 6.9-7.2(4H, m), 7.34(1H, t, J=8.0Hz), 7.83 |
| | | | | | | (1H, dd, J=8.0, 1.4Hz), 8.04(1H, dd, J=8.0, 1.4Hz) |
| | | | | | | Mass: 400(M⁺ + H) |
| (15) | H | Cl | H | H | Me | ¹H NMR(200MHz, DMSO-d₆, δ): 1.3-1.7(4H, m), 1.62 |
| | | | | | | (3H, s), 1.8-2.0(3H, m), 2.3-2.6(4H, m), 2.6-2.8(2H, m), |
| | | | | | | 2.8-3.0(2H, m), 6.9-7.2(4H, m), 7.31(1H, t, J=8.0Hz), |
| | | | | | | 7.81(1H, dd, J=8.0, 1.4Hz), 8.01(1H, dd, J=8.0, |
| | | | | | | 1.4Hz) |
| | | | | | | Mass: 396(M⁺ + H) |
| (16) | H | Cl | H | H | OMe | ¹H NMR(200MHz, DMSO-d₆, δ): 1.3-1.7(4H, m), |
| | | | | | | 1.8-2.0(3H, m), 2.3-2.6(4H, m), 2.6-2.8(2H, m), 2.8-3.0 |
| | | | | | | (2H, m), 3.70(3H, s), 6.80(2H, d, J=8Hz), 6.97(2H, d, J= |
| | | | | | | 8Hz), 7.43(1H, t, J=8Hz), 7.91(1H, dd, J=8.0, |
| | | | | | | 1.4Hz), 8.07(1H, dd, J=8.0, 1.4Hz) |
| | | | | | | Mass: 412(M⁺ + H) |
| (17) | H | Cl | H | H | H | ¹H NMR(200MHz, DMSO-d₆, δ): 1.3-1.7(4H, m), |
| | | | | | | 1.8-2.0(3H, m), 2.3-2.6(4H, m), 2.6-2.8(2H, m), 2.8-3.0 |
| | | | | | | (2H, m), 7.0-7.3(5H, m), 7.42(1H, t, J=8Hz), 7.91(1H, |
| | | | | | | dd, J=8.0, 1.4Hz), 8.07(1H, dd, J=8.0, 1.4Hz) |
| | | | | | | Mass: 382(M⁺ + H) |
| (18) | H | Me | H | H | Me | ¹H NMR(200MHz, DMSO-d₆, δ): 1.3-1.7(4H, m), |
| | | | | | | 1.7-2.0(3H, m), 2.24(3H, s), 2.4-2.6(2H, m), 2.67(3H, |
| | | | | | | s), 2.5-2.8(2H, m), 2.8-3.0(2H, m), 6.96(2H, d, 8Hz), |
| | | | | | | 7.05(2H, d, J=8Hz), 7.30(1H, t, J=8Hz), 7.60(1H, dd, |
| | | | | | | J=7.6, 1.4Hz), 7.93(1H, dd, J=7.6, 1.4Hz) |
| | | | | | | Mass: 376(M⁺ + H) |
| (19) | H | Me | H | H | OMe | ¹H NMR(200MHz, DMSO-d₆, δ): 1.3-1.7(3H, m), |
| | | | | | | 1.7-2.1(4H, m), 2.2-2.4(4H, m), 2.51(3H, s), 2.6-2.8 |
| | | | | | | (2H, m), 2.9-3.1(2H, m), 3.72(3H, s), 6.80(2H, d, 8Hz), |
| | | | | | | 7.01(2H, d, J=8Hz), 7.32(1H, t, J=8Hz), 7.62(1H, dd, |
| | | | | | | J=7.6, 1.4Hz), 7.94(1H, dd, J=7.6, 1.4Hz) |
| | | | | | | Mass: 392(M⁺ + H) |
| (20) | H | Me | H | H | F | ¹H NMR(200MHz, DMSO-d₆, δ): 1.3-1.7(4H, m), |
| | | | | | | 1.8-2.1(3H, m), 2.3-2.5(4H, m), 2.51(3H, s), 2.6-2.8 |
| | | | | | | (2H, m), 2.8-3.1(2H, m), 3.72(3H, s), 7.0-7.3(4H, m), |
| | | | | | | 7.61(1H, t, J=8Hz), 7.93(1H, dd, J=7.6, 1.4Hz), 7.95 |
| | | | | | | (1H, dd, J=7.6, 1.4Hz) |
| | | | | | | Mass: 380(M⁺ + H) |
| (21) | H | OMe | H | H | Me | ¹H NMR(200MHz, DMSO-d₆, δ): 1.3-2.1(7H,m), |
| | | | | | | 2.0-3.0(8H, m), 2.50(3H, s), 4.08(3H, s), 6.9-7.8(7H, m) |
| | | | | | | Mass: 392(M⁺ + H) |
| (22) | H | OMe | H | H | F | ¹H NMR(200MHz, DMSO-d₆, δ): 1.3-2.0(7H, m), |
| | | | | | | 2.2-3.0(8H, m), 2.49(3H, s), 3.90(3H, s), 6.9-7.8(7H, m) |
| | | | | | | Mass: 396(M⁺ + H) |
| (23) | H | OMe | H | H | OMe | ¹H NMR(200MHz, DMSO-d₆, δ): 1.3-2.0(7H, m), |
| | | | | | | 2.1-3.0(8H, m), 3.49(3H, s), 3.71(3H, s), 4.00(3H, s), |
| | | | | | | 6.81(2H, d, J=8Hz), 7.05(2H, d, J=8Hz), 7.2-7.8(3H, |
| | | | | | | m) |
| | | | | | | Mass: 408(M⁺ + H) |
| (24) | Cl | H | H | H | H | ¹H NMR(200MHz, DMSO-d₆, δ): 1.2-1.7(4H, m), |
| | | | | | | 1.8-2.0(4H, m), 2.2-2.5(3H, m), 2.6-3.0(4H, m), 6.9-7.3 |
| | | | | | | (5H, m), 7.61(1H, d, J=8Hz), 7.79(1H, d, J=8Hz), |
| | | | | | | 8.05(1H, s) |
| | | | | | | Mass: 382(M⁺ + H) |
| (25) | Cl | H | H | H | OMe | ¹H NMR(200MHz, DMSO-d₆, δ): 1.2-1.7(4H, m), |
| | | | | | | 1.8-2.0(4H, m), 2.2-2.5(3H, m), 2.6-3.0(4H, m), 3.70 |
| | | | | | | (3H, s), 6.79(2H, d, J=8Hz), 6.96(2H, d, J=8Hz), 7.60 |
| | | | | | | (1H, d, J=8Hz), 7.79(1H, d, J=8Hz), 8.00(1H, s) |
| | | | | | | Mass: 412(M⁺ + H) |
| (26) | Cl | H | H | H | Me | ¹H NMR(200MHz, DMSO-d₆, δ): 1.2-1.7(4H, m), |
| | | | | | | 1.8-2.0(4H, m), 2.2-2.5(3H, m), 2.34(3H, s), 2.6-3.0 |
| | | | | | | (4H, m), 6.95(2H, d, J=8Hz), 7.05(2H, d, J=8Hz), |
| | | | | | | 7.55(1H, d, J=8Hz), 7.75(1H, d, J=8Hz), 8.00(1H, s) |
| | | | | | | Mass: 396(M⁺ + H) |
| (27) | Cl | Cl | H | H | H | ¹H NMR(200MHz, DMSO-d₆, δ): 1.2-2.0(8H, m), |
| | | | | | | 2.2-2.4(3H, m), 2.5-3.0(4H, m), 7.0-7.5(5H, m), 8.0-8.2 |
| | | | | | | (2H, m) |
| | | | | | | Mass: 417(M⁺ + H) |

### EXAMPLE 11

The following compounds are prepared in a similar manner to that of Example 9. If necessary, the starting compounds of them were prepared in similar manners of Preparation 17, Preparation 20 and preparation 23-(2)

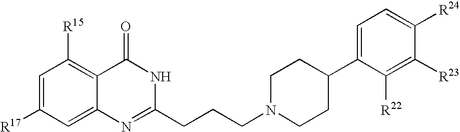

| No. | R¹⁵ | R¹⁷ | R²² | R²³ | R²⁴ | |
|---|---|---|---|---|---|---|
| (1) | Cl | H | H | H | OMe | ¹H NMR(200MHz, DMSO-d₆, δ): 1.2-2.0(8H, m), |
| | | | | | | 2.2-2.4(3H, m), 2.5-2.8(2H, m), 2.8-3.0(2H, m), 3.70 |
| | | | | | | (3H, s), 6.80(2H, d, J=8.0Hz), 7.01(2H, d, J=8.0Hz), |
| | | | | | | 7.3-7.8(3H, m) |
| | | | | | | Mass: 412(M⁺ + H) |
| (2) | Cl | H | H | H | H | ¹H NMR(200MHz, DMSO-d₆, δ): 1.2-2.0(8H, m), |
| | | | | | | 2.2-2.4(3H, m), 2.5-2.8(2H, m), 2.8-3.0(2H, m), 7.0-7.7 |
| | | | | | | (8H, m) |
| | | | | | | Mass: 382(M⁺ + H) |
| (3) | F | H | H | H | OMe | ¹H NMR(200MHz, CDCl₃, δ): 1.8-2.1(4H, m), 2.1-2.3 |
| | | | | | | (4H, m), 2.4-2.6(3H, m), 2.8-3.0(2H, m), 3.19(2H, brd, |
| | | | | | | J=6.2Hz), 3.80(3H, s), 6.89(2H, d, J=8.7Hz), 7.05(1H, |
| | | | | | | dd, J=9.5, 8.4Hz), 7.32(2H, d, J=8.7Hz), 7.41(1H, d, |
| | | | | | | J=8.2Hz), 7.62(1H, dt, J=8.1, 5.5Hz) |
| | | | | | | Mass(API): 396.3(M⁺ + H) |
| (4) | F | H | H | H | F | ¹H NMR(200MHz, CDCl₃, δ): 1.84(2H, brd, J=8.2Hz), |
| | | | | | | 1.97(2H, quint., J=5.7Hz), 2.1-2.4(4H, m), 2.4-2.7(3H, |
| | | | | | | m), 2.8-3.0(2H, m), 3.21(2H, brd, J=6.5Hz), 6.9-7.1 |
| | | | | | | (3H, m), 7.3-7.5(3H, m), 7.62(1H, dt, J=8.2, 5.5Hz) |
| | | | | | | Mass(API): 384.3(M⁺ + H) |

### EXAMPLE 12

The following compounds are prepared in a similar manner to that of Example 9. If necessary, the starting compounds of them were prepared in similar manners of Preparation 17 and Preparation 20.

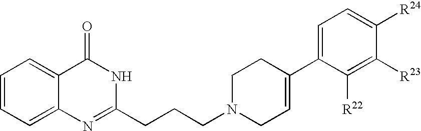

| No. | R²² | R²³ | R²⁴ | |
|---|---|---|---|---|
| (1) | F | H | H | ¹H NMR(200MHz, CDCl₃, δ): 2.05(2H, quint., J=6.4 |
| | | | | Hz), 2.67(2H, t, J=6.2Hz), 2.7-3.0(6H, m), 3.31(2H, q, |
| | | | | J=3.2Hz), 6.02(1H, m), 7.0-7.5(5H, m), 7.6-7.8(2H, |
| | | | | m), 8.25(1H, d, J=7.8Hz), 12.64(1H, br) |
| | | | | Mass(API) 364.3(M⁺ + H) |
| (2) | H | F | H | ¹H NMR(200MHz, CDCl₃, δ): 2.05(2H, quint., J=7.1 |
| | | | | Hz), 2.66(2H, t, J=6.0Hz), 2.7-3.0(6H, m), 3.30(2H, q, |
| | | | | J=3.2Hz), 6.13(1H, m), 6.95(1H, t, J=8.2Hz), 7.1-7.5 |
| | | | | (4H, m), 7.6-7.8(2H, m), 8.23(1H, d, J=7.9Hz), |
| | | | | 12.55(1H, br) |
| | | | | Mass(API): 364.4(M⁺ + H) |
| (3) | OMe | H | H | ¹H NMR(200MHz, CDCl₃, δ): 2.04(2H, quint., J=6.2 |
| | | | | Hz), 2.66(2H, t, J=6.2Hz), 2.7-3.0(6H, m), 3.29(2H, q, |
| | | | | J=2.6Hz), 3.84(3H, s), 5.83(1H, m), 6.88(1H, d, J=8.2 |
| | | | | Hz), 6.95(1H, t, J=7.4Hz), 7.2-7.3(2H, m), 7.42(1H, t, |
| | | | | J=7.3Hz), 7.6-7.8(2H, m), 8.28(1H, d, J=11.2Hz) |
| | | | | Mass(APCI): 376.13(M⁺ + H) |
| (4) | H | OMe | H | ¹H NMR(200MHz, CDCl₃, δ): 2.05(2H, quint., J=7.2 |
| | | | | Hz), 2.66(2H, t, J=6.0Hz), 2.7-3.0(6H, m), 3.30(2H, q, |
| | | | | J=1.6Hz), 3.84(3H, s), 6.10(1H, m), 6.82(1H, dd, |
| | | | | J=8.1, 2.6Hz), 7.00(1H, t, J=2.3Hz), 7.06(1H, d, J=7.9 |
| | | | | Hz), 7.26(1H, t, J=7.9Hz), 7.41(1H, t, J=7.3Hz), |
| | | | | 7.6-7.8(2H, m), 8.23(1H, d, J=7.9Hz) |
| | | | | Mass(APCI): 376.07(M⁺ + H) |
| (5) | H | H | OEt | ¹H NMR(200MHz, CDCl₃, δ): 1.42(3H, t, J=7.0Hz), |
| | | | | 2.04(2H, quint., J=6.0Hz), 2.65(2H, t, J=6.0Hz), |
| | | | | 2.7-3.0(4H, m), 3.29(2H, d, J=3.2Hz), 4.05(2H, q, |
| | | | | J=7.0Hz), 6.01(1H, brs), 6.87(2H, d, J=8.8Hz), 7.3-7.5 |
| | | | | (3H, m), 7.6-7.8(2H, m), 8.23(1H, d, J=7.9Hz) |
| | | | | Mass(API-ES): 390.3(M⁺ + H) |
| (6) | H | H | SMe | ¹H NMR(200MHz, CDCl₃, δ): 2.05(2H, quint., J=6.1 |
| | | | | Hz), 2.49(3H, s), 2.65(2H, t, J=6.0Hz), 2.7-3.0(6H, m), |
| | | | | 3.30(2H, d, J=3.3Hz), 6.08(1H, t, J=3.5Hz), 7.24(2H, |
| | | | | d, J=7.5Hz), 7.3-7.5(3H, m), 7.6-7.8(2H, m), 8.23(1H, |
| | | | | dd, J=7.9, 1.0Hz) |
| | | | | Mass(API-ES): 392.3(M⁺ + H) |
| (7) | H | H | OCF₃ | ¹H NMR(200MHz, CDCl₃, δ): 2.05(2H, quint., J=5.9 |
| | | | | Hz), 2.67(2H, t, J=5.9Hz), 2.7-3.0(6H, m), 3.31(2H, q, |
| | | | | J=3.3Hz), 6.08(1H, t, J=3.5Hz), 7.19(2H, d, J=8.0Hz), |
| | | | | 7.42(1H, t, J=6.6Hz), 7.48(2H, d, J=8.7Hz), 7.6-7.8 |
| | | | | (2H, m), 8.23(1H, dd, J=8.0, 0.9Hz) |
| | | | | MS(APCI): 429.87(M⁺ + H) |
| (8) | H | H | Et | ¹H NMR(200MHz, CDCl₃, δ): 1.24(3H, t, J=7.6Hz), |
| | | | | 2.05(2H, quint., J=6.1Hz), 2.5-3.0(10H, m), 3.29(2H, |
| | | | | q, J=3.3Hz), 6.06(1H, m), 7.17(2H, d, J=8.4Hz), |
| | | | | 7.3-7.5(3H, m), 7.6-7.8(2H, m), 8.23(1H, d, J=8.0Hz) |
| | | | | MS(APCI) 373.73(M⁺ + H) |
| (9) | H | H | N(Me)₂ | ¹H NMR(200MHz, CDCl₃, δ): 2.04(2H, quint., J=6.3 |
| | | | | Hz), 2.64(2H, t, J=6.0Hz), 2.7-2.9(4H, m), 2.95(6H, s), |
| | | | | 3.61(2H, m), 5.98(1H, t, J=3.5Hz), 6.72(2H, d, J=8.9 |
| | | | | Hz), 7.3-7.5(3H, m), 7.6-7.8(2H, m), 8.24(1H, d, J=7.9 |
| | | | | Hz) |
| | | | | MS(API-ES): 389.4(M⁺ + H) |
| (10) | H | H | t-Bu | ¹H NMR(200MHz, CDCl₃, δ): 1.33(9H, s), 2.04(2H, |
| | | | | quint., J=6.1Hz), 2.65(2H, t, J=6.0Hz), 2.8-3.0(4H, m), |
| | | | | 3.30(2H, q, J=3.2Hz), 6.08(1H, brs), 7.3-7.5(5H, m), |
| | | | | 7.63(1H, d, J=6.8Hz), 7.71(1H, t, J=6.7Hz), 8.23(1H, |
| | | | | d, J=7.9Hz) |
| | | | | MS(APCI): 402.00(M⁺ + H) |
| (11) | H | H | Ph | ¹H NMR(200MHz, DMSO-d₆, δ): 1.97(2H, quint., J=6.0 |
| | | | | Hz), 2.4-2.5(4H, m), 2.6-2.8(4H, m), 3.12(2H, brs), |
| | | | | 6.20(1H, m), 7.3-7.5(6H, m), 7.5-7.8(6H, m), 8.06(1H, |
| | | | | d, J=7.9Hz), 12.49(1H, brs) |
| | | | | MS(APCI): 422.07(M⁺ + H) |
| (12) | H | H | OPh | ¹H NMR(200MHz, DMSO-d₆, δ): 1.96(2H, quint., J=6.9 |
| | | | | Hz), 2.4-2.5(4H, m), 2.6-2.7(4H, m), 3.08(2H, brs), |
| | | | | 6.07(1H, brs), 6.95(2H, d, J=8.7Hz), 7.01(2H, d, J=8.3 |
| | | | | Hz), 7.14(1H, t, J=7.4Hz), 7.39(2H, t, J=7.5Hz), 7.40 |
| | | | | (2H, d, J=8.8Hz), 7.59(1H, d, J=7.6Hz), 7.77(1H, t), |
| | | | | 8.04(1H, d, J=7.8Hz), 12.22(1H, brs) |
| | | | | MS(API-ES): 438.3(M⁺ + H) |
| (13) | H | H | Ac | ¹H NMR(200MHz, CDCl₃, δ): 2.06(2H, quint., J=6.1 |
| | | | | Hz), 2.61(3H, s), 2.68(2H, t, J=6.0Hz), 2.8-3.0(4H, m), |
| | | | | 3.33(2H, d, J=3.2Hz), 6.24(1H, t, J=3.6Hz), 7.42(1H, |
| | | | | t), 7.54(2H, d, J=8.6Hz), 7.6-7.8(2H, m), 7.94(2H, d, |
| | | | | J=8.6Hz), 8.22(1H, d, J=7.4Hz) |
| (14) | H | H | Me | ¹H NMR(200MHz, CDCl₃, δ): 2.05(2H, quint., J=6.2 |
| | | | | Hz), 2.35(3H, s), 2.65(2H, t, J=6.0Hz), 2.78-2.93(6H, |
| | | | | m), 3.30(2H, d, J=3.2Hz), 6.06(1H, m), 7.15(2H, d, |
| | | | | J=8.1Hz), 7.35(2H, d, J=8.2Hz), 7.43(1H, d, J=6.5 |
| | | | | Hz), 7.65(1H, t, J=6.9Hz), 7.71(1H, t, J=8.2Hz), 8.24 |
| | | | | (1H, dd, J=8.0, 1.2Hz) |
| | | | | MS(APCI): 360.13(M⁺ + H) |
| (15) | H | H | OMe | ¹H NMR(200MHz, CDCl₃, δ): 2.04(2H, quint., J=6.0 |
| | | | | Hz), 2.65(2H, t, J=6.0Hz), 2.79-2.93(6H, m), 3.29(2H, |
| | | | | d, J=3.2Hz), 3.82(3H, s), 6.01(1H, m), 6.88(2H, d, |
| | | | | J=8.8Hz), 7.37-7.46(3H, m), 7.63(1H, d, J=7.0Hz), |
| | | | | 7.71(1H, t, J=7.8Hz), 8.23(1H, d, J=7.8Hz) |
| | | | | MS(APCI): 376.07(M⁺ + H) |
| (16) | H | H | F | ¹H NMR(200MHz, CDCl₃, δ): 2.05(2H, quint., J=6.1 |
| | | | | Hz), 2.66(2H, t, J=5.9Hz), 2.79-2.93(6H, m), 3.30(2H, |
| | | | | d, J=3.0Hz), 6.03(1H, m), 7.03(2H, t, J=8.7Hz), |
| | | | | 7.37-7.46(3H, m), 7.65(1H, t, J=6.9Hz), 7.71(1H, t, |
| | | | | J=7.5Hz), 8.23(1H, d, J=6.9Hz) |
| | | | | MS(APCI): 364.00(M⁺ + H) |
| (17) | H | H | CF₃ | ¹H NMR(200MHz, CDCl₃, δ): 2.06(2H, quint., J=6.1 |
| | | | | Hz), 2.68(2H, t, J=5.9Hz), 2.83-2.94(6H, m), 3.33(2H, |
| | | | | d, J=3.1Hz), 6.18(1H, m), 7.41(1H, t, J=7.3Hz), |
| | | | | 7.53-7.76(6H, m), 8.23(1H, d, J=6.6Hz) |
| | | | | MS(APCI): 413.93(M⁺ + H) |
| (18) | H | H | CN | ¹H NMR(200MHz, CDCl₃, δ): 2.03(2H, quint., J=6.0 |
| | | | | Hz), 2.68(2H, t, J=5.9Hz), 2.78-2.94(6H, m), 3.33(2H, |
| | | | | q, J=3.3Hz), 6.21(1H, m), 7.43(1H, t, J=8.1Hz), |
| | | | | 7.51-7.72(6H, m), 8.22(1H, dd, J=7.8, 1.1Hz) |
| | | | | MS(APCI): 370.93(M⁺ + H) |
| (19) | H | H | CH₂OH | ¹H-NMR(DMSO-d₆, δ): 1.9-2.1(2H, m), 2.3-2.8(10H, |
| | | | | m), 3.07(2H, d, J=2.8Hz), 4.47(2H, s), 6.08(1H, s), |
| | | | | 7.25(2H, d, J=8.4Hz), 7.34(2H, d, J=8.4Hz), 7.4-7.5 |
| | | | | (1H, m), 7.59(2H, d, J=7.5Hz), 7.7-7.8(1H, m), 8.0-8.1 |
| | | | | (1H, m) |
| | | | | Mass: 376.0(M⁺ + H) |
| (20) | H | H | Cl | ¹H NMR(200MHz, DMSO-d₆, δ): 2.0-2.2(2H, m), |
| | | | | 2.3-2.8(8H, m), 3.0-3.2(2H, m), 6.12(1H, m), 7.0-7.8 |
| | | | | (8H, m) |
| | | | | Mass: 380(M⁺ + H) |

### EXAMPLE 13

The following compounds were prepared in a similar manner to that of Example 9. If necessary, the starting compounds of them were prepared in similar manners of Preparation 17 and Preparation 20.

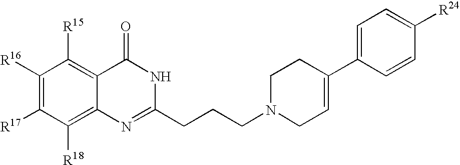

| No. | R¹⁵ | R¹⁶ | R¹⁷ | R¹⁸ | R²⁴ | |
|---|---|---|---|---|---|---|
| (1) | Cl | H | H | H | CN | ¹H NMR(200MHz, DMSO-d₆, δ): d/ppm 1.94(2H, |
| | | | | | | quint., J=6.8Hz), 2.3-2.5(4H, m), 2.5-2.7(4H, m), 3.09 |
| | | | | | | (2H, brs), 6.31(1H, brs), 7.39(1H, d, J=7.6Hz), 7.5-7.7 |
| | | | | | | (4H, m), 7.77(2H, d, J=8.5Hz), 12.23(1H, brs) |
| | | | | | | Mass(APCI): 405.00(M⁺ + H) |
| (2) | Cl | H | H | H | OMe | ¹H NMR(200MHz, DMSO-d₆, δ): 1.8-2.0(2H, m), |
| | | | | | | 2.2-2.8(8H, m), 3.0-3.2(2H, m), 3.74(3H, s), 5.97(1H, |
| | | | | | | m), 6.87(2H, d, J=8.0Hz), 7.35(2H, d, J=8.0Hz), 7.40 |
| | | | | | | (1H, dd, J=7.6, 1.4Hz), 7.51(1H, dd, J=7.6, 1.4Hz), |
| | | | | | | 7.65(1H, t, J=7.6Hz) |
| | | | | | | Mass: 410(M⁺ + H) |
| (3) | F | H | H | H | H | ¹H NMR(200MHz, CDCl₃, δ): 2.04(2H, quint., J=6.1 |
| | | | | | | Hz), 2.66(2H, t, J=6.0Hz), 2.7-3.0(6H, m), 3.31(2H, |
| | | | | | | m), 6.10(1H, m), 7.04(1H, dd, J=10.5, 8.2Hz), 7.2-7.5 |
| | | | | | | (6H, m), 7.63(1H, dt, J=8.1, 5.4Hz) |
| | | | | | | MS(APCI): 364.07(M⁺ + H) |
| (4) | F | H | H | H | OMe | ¹H NMR(200MHz, CDCl₃, δ): 2.03(2H, quint., J=6.7 |
| | | | | | | Hz), 2.65(2H, t, J=6.0Hz), 2.7-2.9(6H, m), 3.29(2H, q, |
| | | | | | | J=3.2Hz), 6.00(1H, t, J=3.5Hz), 6.87(2H, d, J=8.9Hz), |
| | | | | | | 7.04(1H, dd, J=10.5, 8.1Hz), 7.38(2H, d, J=8.9Hz), |
| | | | | | | 7.40(1H, t, J=6.3Hz), 7.62(1H, dt, J=8.2, 5.5Hz) |
| | | | | | | MS(API-ES): 394.4(M⁺ + H) |
| (5) | F | H | H | H | F | ¹H NMR(200MHz, CDCl₃, δ): 2.04(2H, quint., J=6.0 |
| | | | | | | Hz), 2.66(2H, t, J=6.0Hz), 2.7-2.9(6H, m), 3.29(2H, d, |
| | | | | | | J=2.9Hz), 6.03(1H, m), 6.9-7.1(3H, m), 7.3-7.5(3H, |
| | | | | | | m), 7.5-7.7(1H, m) |
| | | | | | | MS(APCI): 381.87(M⁺ + H) |
| (6) | H | H | Cl | H | H | ¹H NMR(200MHz, DMSO-d₆, δ): 1.8-2.0(2H, m), |
| | | | | | | 2.2-2.8(8H, m), 3.0-3.2(2H, m), 6.08(1H, m), 7.1-7.5 |
| | | | | | | (6H, m), 7.65(1H, d, J=2.0Hz), 8.02(1H, d, J=8.0Hz) |
| | | | | | | Mass: 380(M⁺ + H) |
| (7) | H | Cl | H | H | F | ¹H NMR(200MHz, DMSO-d₆, δ): 1.8-2.0(2H, m), |
| | | | | | | 2.2-2.8(8H, m), 2.9-3.1(2H, m), 6.01(1H, m), 7.0-7.5 |
| | | | | | | (5H, m), 7.60(1H, d, J=8Hz), 7.70(1H, dd, J=8.0, 1.6 |
| | | | | | | Hz), 7.93(1H, d, 1.6Hz) |
| | | | | | | Mass: 398(M⁺ + H) |
| (8) | H | Cl | H | H | OMe | ¹H NMR(200MHz, DMSO-d₆, δ): 1.8-2.0(2H, m), |
| | | | | | | 2.2-2.8(8H, m), 2.9-3.1(2H, m), 5.94(1H, m), 6.86(2H, |
| | | | | | | d, J=8Hz), 7.28(2H, d, J=8Hz), 7.59(1H, d, J=8Hz), |
| | | | | | | 7.75(1H, dd, J=8.0, 1.6Hz), 7.93(1H, d, 1.6Hz) |
| | | | | | | Mass: 410(M⁺ + H) |
| (9) | H | Cl | H | H | H | ¹H NMR(200MHz, DMSO-d₆, δ): 1.8-2.0(2H, m), |
| | | | | | | 2.2-2.8(8H, m), 2.9-3.1(2H, m), 6.05(1H, m), 7.1-7.5 |
| | | | | | | (5H, m), 7.61(1H, d, J=8Hz), 7.78(1H, dd, J=8.0, 1.6 |
| | | | | | | Hz), 8.01(1H, d, 1.6Hz) |
| | | | | | | Mass: 380(M⁺ + H) |
| (10) | H | Cl | H | Cl | H | ¹H NMR(200MHz, DMSO-d₆, δ): 1.8-2.0(2H, m), |
| | | | | | | 2.2-2.8(8H, m), 3.0-3.2(2H, m), 5.99(1H, m), 7.2-7.4 |
| | | | | | | (5H, m), 7.80(1H, d, J=1.4Hz), 8.02(1H, d, J=1.2Hz) |
| | | | | | | Mass: 415(M⁺ + H) |
| (11) | H | Cl | H | Cl | OMe | ¹H NMR(200MHz, DMSO-d₆, δ): 1.8-2.0(2H, m), |
| | | | | | | 2.2-2.8(8H, m), 3.0-3.2(2H, m), 3.74(3H, s), 5.88(1H, |
| | | | | | | m), 6.85(2H, d, J=8Hz), 7.22(2H, J=8Hz), 7.88(1H, d, |
| | | | | | | J=1.5Hz), 8.11(1h, d, J=1.5Hz) |
| | | | | | | Mass: 445(M⁺ + H) |
| (12) | H | Cl | H | Cl | F | ¹H NMR(200MHz, DMSO-d₆, δ): 1.8-2.0(2H, m), |
| | | | | | | 2.2-2.8(8H, m), 3.0-3.2(2H, m), 5.95(1H, m), 6.9-7.3 |
| | | | | | | (4H, m), 7.86(1H, d, J=1.5Hz), 8.00(1H, d, J=1.5Hz) |
| | | | | | | Mass: 433(M⁺ + H) |
| (13) | H | Cl | H | Cl | Me | ¹H NMR(200MHz, DMSO-d₆, δ): 1.8-2.0(2H, m), |
| | | | | | | 2.2-2.8(5H, m), 2.48(3H, s), 3.0-3.2(2H, m), 5.95(1H, |
| | | | | | | m), 7.0-7.3(4H, m), 8.01(1H, d, J=1.5Hz), 8.06(1H, d, |
| | | | | | | J=1.5Hz) |
| | | | | | | Mass: 429(M⁺ + H) |
| (14) | H | F | H | H | F | ¹H NMR(200MHz, DMSO-d₆, δ): 2.0-2.2(2H, m), |
| | | | | | | 2.3-2.9(8H, m), 3.0-3.2(2H, m), 6.04(1H, m), 7.1-7.3 |
| | | | | | | (2H, m), 7.3-7.5(2H, m), 7.6-7.9(3H, m) |
| | | | | | | Mass: 382(M⁺ + H) |
| (15) | H | F | H | H | OMe | ¹H NMR(200MHz, DMSO-d₆, δ): 2.0-2.2(2H, m), |
| | | | | | | 2.3-2.8(8H, m), 3.0-3.2(2H, m), 3.74(3H, s), 5.97(1H, |
| | | | | | | m), 6.87(2H, d, J=8Hz), 7.33(2H, d, J=8Hz), 7.6-7.9 |
| | | | | | | (3H, m) |
| | | | | | | Mass: 394(M⁺ + H) |
| (16) | H | F | H | H | Cl | ¹H NMR(200MHz, DMSO-d₆, δ): 2.0-2.2(2H, m), |
| | | | | | | 2.3-2.8(8H, m), 3.0-3.2(2H, m), 6.12(1H, m), 7.0-7.8 |
| | | | | | | (7H, m) |
| | | | | | | Mass: 398(M⁺ + H) |
| (17) | H | Me | H | H | H | ¹H NMR(200MHz, DMSO-d₆, δ): 2.0-2.2(2H, m), 2.32 |
| | | | | | | (3H, s), 2.8-3.8(10H, m), 6.16(1H, m), 7.2-7.9(9H, m) |
| | | | | | | Mass: 360(M⁺ + H) |
| (18) | H | Me | H | H | F | ¹H NMR(200MHz, DMSO-d₆, δ): 2.0-2.2(2H, m), 2.51 |
| | | | | | | (3H, s), 2.8-3.8(10H, m), 6.13(1H, m), 7.1-7.7(6H, m), |
| | | | | | | 7.86(1H, s) |
| | | | | | | Mass: 378(M⁺ + H) |
| (19) | H | H | H | I | H | ¹H NMR(DMSO-d₆, δ): 1.80-2.20(2H, m), 2.30-2.90 |
| | | | | | | (8H, m), 3.10(2H, d, J=3.1Hz), 6.06(1H, s), 7.00-7.60 |
| | | | | | | (6H, m), 8.03(1H, dd, J=1.4Hz, J=7.8Hz), 8.30(1H, dd, |
| | | | | | | J=1.4Hz, J=7.8Hz) |
| | | | | | | Mass(APCI): 470.20(M⁺ + H) |
| (20) | H | H | H | Br | H | ¹H NMR(DMSO-d₆, δ): 1.80-2.10(2H, m), 2.20-2.90 |
| | | | | | | (8H, m), 3.10(2H, d, J=2.7Hz), 6.07(1H, s), 7.10-7.60 |
| | | | | | | (6H, m), 7.90-8.20(2H, m), 12.42(1H, brs) |
| | | | | | | Mass(APCI): 424.33(M⁺ + H) |
| (21) | H | H | H | Et | H | ¹H NMR(DMSO-d₆, δ): 1.24(3H, t, J=7.4Hz), 1.80- |
| | | | | | | 2.10(2H, m), 2.20-2.80(8H, m), 3.00(2H, q, J=7.4Hz), |
| | | | | | | 6.11(1H, s), 7.10-7.50(6H, m), 7.63(1H, dd, J=1.6, 7.3 |
| | | | | | | Hz), 7.91(1H, dd, J=1.6, 7.9Hz) |
| | | | | | | Mass(APCI): 373.49(M⁺ + H) |
| (22) | H | H | H | Cl | OMe | ¹H-NMR(DMSO-d₆, δ): 1.9-2.1(2H, m), 2.46(2H, s), |
| | | | | | | 2.5-2.8(6H, m), 3.05(2H, s), 3.74(3H, s), 5.95(1H, s), |
| | | | | | | 6.86(2H, d, J=8.7Hz), 7.28(2H, d, J=8.7Hz), 7.38(1H, |
| | | | | | | t, J=7.8Hz), 7.81(1H, d, J=7.8Hz), 7.99(1H, d, J=7.8 |
| | | | | | | Hz) |
| | | | | | | Mass: 410.0(M⁺ + H) |
| (23) | H | H | H | Cl | H | ¹H-NMR(DMSO-d₆, δ): 1.9-2.1(2H, m), 2.29(2H, s), |
| | | | | | | 2.45-2.8(6H, m), 3.07(2H, d, J=3.1Hz), 6.06(1H, s), |
| | | | | | | 7.2-7.4(6H, m), 7.90(1H, dd, J=7.8, 1.5Hz), 7.99(1H, |
| | | | | | | dd, J=7.8, 1.4Hz), 12.46(1H, brs) |
| | | | | | | Mass: 380.1(M⁺ + H) |
| (24) | H | H | H | Cl | CF₃ | ¹H-NMR(DMSO-d₆, δ): 1.9-2.1(2H, m), 2.3-2.5(2H, |
| | | | | | | m), 2.5-2.8(6H, m), 3.10(2H, d, J=2.6Hz), 6.24(1H, s), |
| | | | | | | 7.36(1H, t, J=7.8Hz), 7.56(2H, d, J=8.3Hz), 7.66(2H, |
| | | | | | | d, J=8.3Hz), 7.91(1H, dd, J=7.8, 1.4Hz), 7.98(1H, dd, |
| | | | | | | J=7.8, 1.4Hz) |
| | | | | | | Mass: 448.1(M⁺ + H) |
| (25) | H | H | H | Cl | CH2OH | ¹H-NMR(DMSO-d₆, δ): 1.9-2.1(2H, m), 2.3-2.5(2H, |
| | | | | | | m), 2.5-2.8(4H, m), 3.07(2H, d, J=2.9Hz), 4.46(2H, d, |
| | | | | | | J=5.0Hz), 5.12(1H, t, J=5.4Hz), 6.05(1H, s), 7.24(2H, |
| | | | | | | d, J=8.4Hz), 7.31(2H, d, J=8.4Hz), 7.38(1H, t, J=7.9 |
| | | | | | | Hz), 7.90(1H, dd, J=7.9, 1.4Hz), 7.99(1H, dd, J=7.9, |
| | | | | | | 1.4Hz) |
| | | | | | | Mass: 410.0(M⁺ + H) |
| (26) | H | H | H | Cl | F | ¹H NMR(200MHz, DMSO-d₆, δ): 1.8-2.1(2H, m), |
| | | | | | | 2.2-2.8(8H, m), 3.3(2H, br.s), 6.03(1H, m), 7.0-7.2(2H, |
| | | | | | | m), 7.3-7.6(2H, m), 7.42(1H, t, J=8.0Hz), 7.90(1H, dd, |
| | | | | | | J=8.0, 1.4Hz), 7.99(1H, dd, J=8.0, 1.4Hz) |
| | | | | | | Mass: 398(M⁺ + H) |
| (27) | H | H | H | Cl | OMe | ¹H NMR(200MHz, DMSO-d₆, δ): 1.8-2.1(2H, m), |
| | | | | | | 2.2-2.8(8H, m), 3.1(2H, br.s), 3.74(3H, s), 5.98(1H, m), |
| | | | | | | 6.87(2H, d, J=8.8Hz), 7.28(1Ht, J=8.2Hz), 7.29(2H, |
| | | | | | | d, J=8.8Hz), 7.79(1H, dd, J=8.8, 1.4Hz), 7.96(1H, dd, |
| | | | | | | J=8.8, 1.4Hz) |
| | | | | | | Mass: 410(M⁺ + H) |
| (28) | H | H | H | Cl | Me | ¹H NMR(200MHz, DMSO-d₆, δ): 1.8-2.1(2H, m), 2.1 |
| | | | | | | (3H, s), 2.2-2.8(8H, m), 3.1(2H, br.s), 6.03(1H, m), 7.11 |
| | | | | | | (2H, d, J=8.8Hz), 7.22(2H, d, J=8.8Hz), 7.29(1H, t, |
| | | | | | | J=8.8Hz), 7.81(1H, dd, J=8.8, 1.4Hz), 7.96(1H, dd, |
| | | | | | | J=8.8, 1.4Hz) |
| | | | | | | Mass: 394(M⁺ + H) |
| (29) | H | H | H | Cl | Cl | ¹H NMR(200MHz, DMSO-d₆, δ): 1.8-2.1(2H, m), |
| | | | | | | 2.3-2.8(8H, m), 3.05(2H, br.s), 6.13(1H, m), 7.2-7.5 |
| | | | | | | (5H, m), 7.83(1H, dd, J=8.0, 1.4Hz), 7.96(1H, dd, |
| | | | | | | J=8.0, 1.4Hz) |
| | | | | | | Mass: 415(M⁺ + H) |
| (30) | H | H | H | Cl | H | ¹H NMR(200MHz, DMSO-d₆, δ): 1.8-2.1(2H, m), |
| | | | | | | 2.3-2.8(8H, m), 3.05(2H, br.s), 6.07(1H, m), 7.2-7.5 |
| | | | | | | (5H, m), 7.86(1H, dd, J=8.0, 1.4Hz), 7.97(1H, dd, |
| | | | | | | J=8.0, 1.4Hz) |
| | | | | | | Mass: 380(M⁺ + H) |
| (31) | H | H | H | Me | CF₃ | ¹H NMR(200MHz, DMSO-d₆, δ): 1.8-2.0(2H, m), 2.50 |
| | | | | | | (3H, s), 2.3-2.7(8H, m), 3.1-3.3(2H, m), 6.28(1H, br.s), |
| | | | | | | 7.29(1H, t, J=8.0Hz), 7.5-7.8(5H, m), 7.88(1H, d, J=8 |
| | | | | | | Hz) |
| | | | | | | Mass: 428(M⁺ + H) |
| (32) | H | H | H | Me | Cl | ¹H NMR(200MHz, DMSO-d₆, δ): 1.8-2.0(2H, m), 2.50 |
| | | | | | | (3H, s), 2.4-2.8(8H, m), 3.0-3.2(2H, m), 6.15(1H, m), |
| | | | | | | 7.2-7.6(5H, m), 7.60(1H, dd, J=7.6, 1.4Hz), 7.88(1H, |
| | | | | | | dd, J=7.6, 1.4Hz) |
| | | | | | | Mass: 394(M⁺ + H) |
| (33) | H | H | H | Me | Me | ¹H NMR(200 MHz, DMSO-d₆, δ): 1.8-2.0(2H, m), 2.40 |
| | | | | | | (3H, s), 2.59(3H, s), 2.4-2.8(8H, m), 3.0-3.2(2H, m), |
| | | | | | | 6.06(1H, m), 7.11(2H, d, J=8Hz), 7.24(2H, d, J=8Hz), |
| | | | | | | 7.30(1H, t, J=8Hz), 7.61(1H, dd, J=7.6, 1.4Hz), 7.89 |
| | | | | | | (1H, dd, J=7.6, 1.4Hz) |
| | | | | | | Mass: 374(M⁺ + H) |
| (34) | H | H | H | Me | OMe | ¹H NMR(200MHz, DMSO-d₆, δ): 1.8-2.0(2H, m), 2.59 |
| | | | | | | (3H, s), 2.4-2.8(8H, m), 3.0-3.2(2H, m), 3.74(3H, s), |
| | | | | | | 5.99(1H, m), 6.87(2H, d, J=8Hz), 7.25(2H, d, J=8Hz), |
| | | | | | | 7.25(1H, t, J=8Hz), 7.60(1H, dd, J=7.6, 1.4Hz), 7.89 |
| | | | | | | (1H, dd, J=7.6, 1.4Hz) |
| | | | | | | Mass: 389(M⁺ + H) |
| (35) | H | H | H | Me | F | ¹H NMR(200MHz, DMSO-d₆, δ): 1.8-2.0(2H, m), 2.59 |
| | | | | | | (3H, s), 2.4-2.8(8H, m), 3.0-3.2(2H, m), 6.05(1H, m), |
| | | | | | | 7.0-7.5(5H, m), 7.80(1H, dd, J=7.6, 1.4Hz), 7.95(1H, |
| | | | | | | dd, J=7.6, 1.4Hz) |
| | | | | | | Mass: 378(M⁺ + H) |
| (36) | H | H | H | OMe | H | ¹H NMR(200MHz, DMSO-d₆, δ): 1.8-2.0(2H, m), |
| | | | | | | 2.4-2.8(8H, m), 3.0-3.2(2H, m), 3.89(3H, m), 6.11(1H, |
| | | | | | | m), 7.1-7.7(7H, m) |
| | | | | | | Mass: 376(M⁺ + H) |
| (37) | H | H | H | OMe | CF₃ | ¹H NMR(200MHz, DMSO-d₆, δ): 1.8-2.0(2H, m), |
| | | | | | | 2.6-2.9(8H, m), 3.0-3.2(2H, m), 3.88(3H, m), 6.29(1H, |
| | | | | | | m), 7.2-7.8(7H, m) |
| | | | | | | Mass: 444(M⁺ + H) |
| (38) | H | H | H | OMe | Cl | ¹H NMR(200MHz, DMSO-d₆, δ): 1.8-2.0(2H, m), |
| | | | | | | 2.4-2.8(8H, m), 3.0-3.2(2H, m), 3.88(3H, m), 6.15(1H, |
| | | | | | | m), 7.2-7.7(7H, m) |
| | | | | | | Mass: 410(M⁺ + H) |
| (39) | H | H | H | OMe | Me | ¹H NMR(200MHz, DMSO-d₆, δ): 1.8-2.0(2H, m), 2.27 |
| | | | | | | (3H, s), 2.4-2.8(8H, m), 3.0-3.2(2H, m), 3.88(3H, m), |
| | | | | | | 6.07(1H, m), 7.1-7.7(7H, m) |
| | | | | | | Mass: 390(M⁺ + H) |
| (40) | H | H | H | OMe | OMe | ¹H NMR(200MHz, DMSO-d₆, δ): 1.8-2.0(2H, m), 2.27 |
| | | | | | | (3H, s), 2.4-2.8(8H, m), 3.0-3.2(2H, m), 3.88(3H, m), |
| | | | | | | 4.09(3H, s), 5.99(1H, m), 6.8-7.7(7H, m) |
| | | | | | | Mass: 406(M⁺ + H) |
| (41) | H | H | H | Cl | CN | ¹H NMR(200MHz, DMSO-d₆, δ): d/ppm 1.98(2H, |
| | | | | | | quint., J=6.9Hz), 2.3-2.8(8H, m), 3.11(2H, d, J=2.9 |
| | | | | | | Hz), 6.29(1H, brs), 7.36(1H, t, J=7.9Hz), 7.53(2H, d, |
| | | | | | | J=8.5Hz), 7.77(2H, d, J=8.4Hz), 7.90(1H, d, J=7.8 |
| | | | | | | Hz), 7.97(1H, d, J=7.9Hz), 12.49(1H, br) |
| | | | | | | Mass(APCI): 405.00(M⁺ + H) |
| (42) | H | H | H | Cl | Ac | ¹H NMR(200MHz, DMSO-d₆, δ): d/ppm 1.99(2H, |
| | | | | | | quint., J=6.9Hz), 2.3-2.8(8H, m), 3.11(2H, d, J=2.8 |
| | | | | | | Hz), 6.26(1H, brs), 7.37(1H, t, J=7.8Hz), 7.49(2H, d, |
| | | | | | | J=8.4Hz), 7.90(2H, d, J=8.4Hz), 7.91(1H, d, J=7.8 |
| | | | | | | Hz), 7.98(1H, d, J=7.9Hz), 12.44(1H, br) |
| | | | | | | Mass(API-ES): 422.2(M⁺ + H) |

### EXAMPLE 14

The following compounds are prepared in a similar manner to that of Example 9. If necessary, the starting compounds of them were prepared in similar manners of Preparation 17 and Preparation 20.

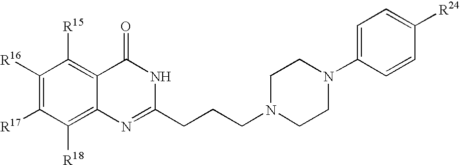

| No. | R¹⁵ | R¹⁶ | R¹⁷ | R¹⁸ | R²⁴ | |
|---|---|---|---|---|---|---|
| (1) | H | H | H | H | H | ¹H NMR(200MHz, CDCl₃, δ): 2.05(2H, quint., J=6.0 |
| | | | | | | Hz), 2.62(2H, t, J=5.8Hz), 2.78(4H, t, J=5.0Hz), |
| | | | | | | 2.8-3.0(2H, m), 3.45(4H, t, J=5.0Hz), 6.87(1H, t, J=7.2 |
| | | | | | | Hz), 6.98(2H, d, J=7.8Hz), 7.28(2H, t, J=8.0Hz), 7.42 |
| | | | | | | (1H, t, J=7.4Hz), 7.6-7.8(2H, m), 8.23(1H, d, J=8.0 |
| | | | | | | Hz), 12.92(1H, brs) |
| | | | | | | Mass(APCI): 349.20(M⁺ + H) |
| (2) | H | H | H | Cl | H | ¹H NMR(200MHz, DMSO-d₆, δ): 1.8-2.0(2H, m), |
| | | | | | | 2.3-2.8(8H, m), 3.0-3.2(2H, m), 6.7-7.2(5H, m), 7.33 |
| | | | | | | (1H, t, J=8.0Hz), 7.85(1H, dd, J=8.0, 1.4Hz), 8.01(1H, |
| | | | | | | dd, J=8.0, 1.4Hz) |
| | | | | | | Mass: 383(M⁺ + H) |
| (3) | H | H | H | Cl | OMe | ¹H NMR(200MHz, DMSO-d₆, δ): 1.8-2.0(2H, |
| | | | | | | m), 2.3-3.0(12H, m), 3.67(3H, s), 6.8-7.0(4H, m), 7.36 |
| | | | | | | (1H, t, J=8.0Hz), 7.88(1H, dd, J=8.0, 1.4Hz), 7.99(1H, |
| | | | | | | dd, J=8.0, 1.4Hz) |
| | | | | | | Mass: 413(M⁺ + H) |
| (4) | H | H | H | Cl | CN | ¹H NMR(200MHz, DMSO-d₆, δ): 1.8-2.0(2H, |
| | | | | | | m), 2.3-2.9(8H, m), 3.1-3.3(4H, m), 6.97(2H, d, J=8.0 |
| | | | | | | Hz), 7.06(1H, t, J=8.0Hz), 7.55(2H, d, J=8.0Hz), 8.00 |
| | | | | | | (1H, dd, J=8.0, 1.2Hz), 8.02(1H, dd, J=8.0, 1.2Hz) |
| | | | | | | Mass: 408(M⁺ + H) |
| (5) | H | H | H | Cl | Me | ¹H NMR(200MHz DMSO-d₆, δ): 1.8-2.0(2H, m), 2.18 |
| | | | | | | (3H, s), 2.1-2.9(8H, m), 2.8-3.0(4H, m), 6.75(2H, d, |
| | | | | | | J=8.0Hz), 7.00(2H, d, J=8.0Hz), 7.40(1H, t, J=8.0Hz), |
| | | | | | | 7.91(1H, dd, J=8.0, 1.2Hz), 8.01(1H, dd, J=8.0, 1.2 |
| | | | | | | Hz) |
| | | | | | | Mass: 398(M⁺ + H) |
| (6) | H | H | H | Cl | Ph | ¹H NMR(200MHz, DMSO-d₆, δ): 1.8-2.0(2H, |
| | | | | | | m), 2.3-3.2(12H, m), 6.9-7.7(10H, m), 7.80(1H, dd, |
| | | | | | | J=8.0, 1.2Hz), 7.95(1H, dd, J=8.0, 1.2Hz) |
| | | | | | | Mass: 459(M⁺ + H) |
| (7) | H | H | H | Cl | F | ¹H NMR(200MHz, DMSO-d₆, δ): 1.8-2.0(2H, m), |
| | | | | | | 2.3-3.2(12H, m), 6.7-7.1(4H, m), 7.35(1H, t, J=8.0Hz), |
| | | | | | | 7.86(1H, dd, J=8.0, 1.2Hz), 8.00(1H, dd, J=8.0, 1.2Hz) |
| | | | | | | Mass: 401(M⁺ + H) |
| (8) | H | H | H | Cl | NO₂ | ¹H NMR(200MHz, DMSO-d₆, δ): 1.8-2.0(2H, m), |
| | | | | | | 2.3-3.0(12H, m), 6.99(2H, d, J=9.6Hz), 7.39(1H, t, |
| | | | | | | J=7.9Hz), 7.90(1H, dd, J=7.9, 1.6Hz), 8.0-8.2(3H, m) |
| | | | | | | Mass: 428(M⁺ + H) |
| (9) | H | H | H | Cl | CF₃ | ¹H NMR(200MHz, DMSO-d₆, δ): 1.8-2.0(2H, m), |
| | | | | | | 2.3-3.0(8H, m), 3.0-3.2(4H, m), 7.00(2H, d, J=8.6Hz), |
| | | | | | | 7.3-7.6(3H, m), 7.91(1H, dd, J=7, 9, 1.4Hz), 8.02(1H, |
| | | | | | | dd, J=7.9, 1.4Hz) |
| | | | | | | Mass: 451(M⁺ + H) |
| (10) | H | H | H | Me | F | ¹H NMR(200MHz, DMSO-d₆, δ): 1.8-2.0(2H, m), |
| | | | | | | 2.2-2.8(8H, m), 2.52(3H, s), 2.8-3.0(2H, m), 6.8-7.1 |
| | | | | | | (4H, m), 7.31(1H, t, J=8Hz), 7.62(1H, d, J=8Hz), 7.90 |
| | | | | | | (1H, d, J=8Hz) |
| | | | | | | Mass: 381(M⁺ + H) |
| (11) | H | H | H | Me | Cl | ¹H NMR (200MHz, DMSO-d₆, δ): 1.8-2.0(2H, m), |
| | | | | | | 2.2-2.8(8H, m), 2.52(3H, s), 2.8-3.0(2H, m), 6.90(2H, |
| | | | | | | d, J=8Hz), 7.22(2H, d, J=8Hz), 7.28(1H, t, J=8Hz), |
| | | | | | | 7.59(1H, d, J=8Hz), 7.88(1H, d, J=8Hz) |
| | | | | | | Mass: 397(M⁺ + H) |
| (12) | H | H | H | OMe | Cl | ¹H NMR(200MHz, DMSO-d₆, δ): 1.6-2.0(4H, m), |
| | | | | | | 2.2-2.8(5H, m), 3.0-3.3(4H, m), 3.88(3H, s), 6.8-7.7 |
| | | | | | | (7H, m) |
| | | | | | | Mass: 413(M⁺ + H) |
| (13) | H | H | H | OMe | F | ¹H NMR(200MHz, DMSO-d₆, δ): 1.6-2.0(4H, m), |
| | | | | | | 2.2-2.8(5H, m), 3.0-3.3(4H, m), 3.88(3H, s), 6.8-7.7 |
| | | | | | | (7H, m) |
| | | | | | | Mass: 397(M⁺ + H) |
| (14) | H | H | H | OMe | Cl | ¹H NMR(200MHz, DMSO-d₆, δ): 1.8-2.0(2H, m), |
| | | | | | | 2.2-3.0(8H, m), 3.0-3.2(2H, m), 6.8-7.0(2H, m), 7.1-7.3 |
| | | | | | | (2H, m), 7.4-7.8(3H, m) |
| | | | | | | Mass: 401(M⁺ + H) |
| (15) | H | Cl | H | H | Cl | ¹H NMR(200MHz, DMSO-d₆, δ): 1.8-2.0(2H, m), |
| | | | | | | 2.2-2.8(8H, m), 2.9-3.1(2H, m), 6.88(2H, d, J=8Hz), |
| | | | | | | 7.18(2H, d, J=8Hz), 7.55(1H, d, J=8Hz), 7.81(1H, d, |
| | | | | | | J=8Hz), 7.99(1H, s) |
| | | | | | | Mass: 417(M⁺ + H) |
| (16) | H | Cl | H | H | F | ¹H NMR(200MHz, DMSO-d₆, δ): 1.8-2.0(2H, m), |
| | | | | | | 2.2-2.8(8H, m), 2.9-3.1(2H, m), 6.7-7.1(4H, m), 7.59 |
| | | | | | | (1H, d, J=8Hz), 7.79(1H, d, J=8Hz), 8.52(1H, s) |
| | | | | | | Mass: 401(M⁺ + H) |
| (17) | H | Cl | H | H | H | ¹H NMR(200MHz, DMSO-d₆, δ): 1.8-2.0(2H, m), |
| | | | | | | 2.2-2.8(8H, m), 2.9-3.1(2H, m), 6.7-7.2(5H, m), 7.61 |
| | | | | | | (1H, d, J=8Hz), 7.80(1H, d, J=8Hz), 8.32(1H, s) |
| | | | | | | Mass: 383(M⁺ + H) |
| (18) | H | Cl | H | H | NO₂ | ¹H NMR(200MHz, DMSO-d₆, δ): 1.8-2.0(2H, m), |
| | | | | | | 2.2-3.2(10H, m), 6.8-7.1(2H, m), 7.62(1H, d, J=8Hz), |
| | | | | | | 7.80(1H, d, J=8Hz), 7.9-8.1(3H, m) |
| | | | | | | Mass: 428(M⁺ + H) |
| (19) | H | Cl | H | H | Ph | ¹H NMR(200MHz, DMSO-d₆, δ): 1.8-2.0(2H, m), |
| | | | | | | 2.2-3.2(10H, m), 6.8-7.8(10H, m), 7.81(1H, d, J=8Hz), |
| | | | | | | 7.98(1H, s) |
| | | | | | | Mass: 459(M⁺ + H) |
| (20) | Cl | H | H | H | Cl | ¹H NMR(200MHz, DMSO-d₆, δ): 1.7-2.0(2H, m), |
| | | | | | | 2.3-2.7(8H, m), 2.9-3.2(4H, m), 6.89(2H, d, J=8Hz), |
| | | | | | | 7.26(2H, d, J=8Hz), 7.3-7.7(3H, m) |
| | | | | | | Mass: 417(M⁺ + H) |
| (21) | H | H | H | H | Br | ¹H NMR(200MHz, DMSO-d₆, δ): 1.7-2.0(2H, m), |
| | | | | | | 2.3-2.7(8H, m), 3.1-3.3(4H, m), 6.84(2H, d, J=9.2Hz), |
| | | | | | | 7.32(2H, d, J=9.2Hz), 7.37(1H, t, J=9.0Hz), 7.71(1H, |
| | | | | | | d, J=9.0Hz), 7.78(1H, td, J=9.0, 1.2Hz), 8.04(1H, dd, |
| | | | | | | J=9.0, 1.2Hz) |
| | | | | | | Mass: 428(M⁺ + H) |
| (22) | H | H | H | H | Cl | ¹H NMR(200MHz, DMSO-d₆, δ): 1.7-2.0(2H, m), |
| | | | | | | 2.3-2.7(8H, m), 3.1-3.3(4H, m), 6.88(2H, d, J=9.2Hz), |
| | | | | | | 7.35(2H, d, J=9.2Hz), 7.38(1H, t, J=9.0Hz), 7.71(1H, |
| | | | | | | d, J=9.0Hz), 7.78(1H, td, J=9.0, 1.2Hz), 8.05(1H, dd, |
| | | | | | | J=9.0, 1.2Hz) |
| | | | | | | Mass: 383(M⁺ + H) |
| (23) | H | H | H | H | F | ¹H NMR(200MHz, DMSO-d₆, δ): 1.7-2.0(2H, m), |
| | | | | | | 2.3-2.7(8H, m), 3.1-3.3(4H, m), 6.8-7.0(4H, m), 7.40 |
| | | | | | | (1H, t, J=9.0Hz), 7.79(1H, d, J=9.0Hz), 7.82(1H, td, |
| | | | | | | J=9.0, 1.2Hz), 8.06(1H, dd, J=9.0, 1.2Hz) |
| | | | | | | Mass: 367(M⁺ + H) |
| (24) | H | H | H | H | OMe | ¹H NMR(200MHz, DMSO-d₆, δ): 1.7-2.0(2H, m), |
| | | | | | | 2.3-2.7(8H, m), 2.8-3.0(4H, m), 3.67(3H, m), 6.6-7.0 |
| | | | | | | (4H, m), 7.40(1H, t, J=9.0Hz), 7.56(1H, d, J=9.0Hz), |
| | | | | | | 7.70(1H, td, J=9.0, 1.2Hz), 8.05(1H, dd, J=9.0, 1.2Hz) |
| | | | | | | Mass: 379(M⁺ + H) |
| (25) | H | H | H | H | OH | ¹H NMR(200MHz, DMSO-d₆, δ): 1.7-2.0(2H, m), |
| | | | | | | 2.3-2.7(8H, m), 2.8-3.0(4H, m), 6.6-7.0(4H, m), 7.43 |
| | | | | | | (1H, t, J=9.0Hz), 7.58(1H, d, J=9.0Hz), 7.76(1H, td, |
| | | | | | | J=9.0, 1.2Hz), 8.06(1H, dd, J=9.0, 1.2Hz) |
| | | | | | | Mass: 365(M⁺ + H) |
| (26) | H | H | H | H | NO₂ | ¹H NMR(200MHz, DMSO-d₆, δ): 1.7-2.0(2H, m), |
| | | | | | | 2.3-2.7(8H, m), 3.2-3.5(4H, m), 7.02(2H, d, J=8.0Hz), |
| | | | | | | 7.33(1H, t, J=9.0Hz), 7.52(1H, d, J=9.0Hz), 7.69(1H, |
| | | | | | | td, J=9.0, 1.2Hz), 8.06(1H, dd, J=9.0, 1.2Hz), 8.07(2H, |
| | | | | | | d, J=8.0Hz) |
| | | | | | | Mass: 394(M⁺ + H) |
| (27) | H | H | H | H | NH₂ | ¹H NMR(200MHz, DMSO-d₆, δ): 1.7-2.0(2H, m), |
| | | | | | | 2.3-2.7(8H, m), 2.8-3.0(4H, m), 6.44(2H, d, J=8.0Hz), |
| | | | | | | 6.81(2H, d, J=8Hz), 7.39(1H, t, J=9.0Hz), 7.57(1H, d, |
| | | | | | | J=9.0Hz), 7.75(1H, td, J=9.0, 1.2Hz), 8.06(1H, dd, |
| | | | | | | J=9.0, 1.2Hz) |
| | | | | | | Mass: 364(M⁺ + H) |
| (28) | H | H | H | H | N(Me)₂ | ¹H NMR(200MHz, DMSO-d₆, δ): 1.7-2.0(2H, m), |
| | | | | | | 2.3-2.7(8H, m), 2.8-3.0(4H, m), 6.5-7.0(4H, m), 7.39 |
| | | | | | | (1H, t, J=9.0Hz), 7.57(1H, d, J=9.0Hz), 7.75(1H, td, |
| | | | | | | J=9.0, 1.2Hz), 8.06(1H, dd, J=9.0, 1.2Hz) |
| | | | | | | Mass: 392(M⁺ + H) |
| (29) | H | H | H | H | NHBz | ¹H NMR(200MHz, DMSO-d₆, δ): 1.7-2.0(2H, m), |
| | | | | | | 2.3-2.7(8H, m), 3.0-3.2(4H, m), 6.7-8.2(14H, m) |
| | | | | | | Mass: 467(M⁺ + H) |
| (30) | H | H | H | H | NHAc | ¹H NMR(200MHz, DMSO-d₆, δ): 1.7-2.0(2H, m), 1.98 |
| | | | | | | (3H, s), 2.3-2.7(8H, m), 2.8-3.0(4H, m), 6.81(2H, d, |
| | | | | | | J=8Hz), 7.38(2H, d, J=8Hz), 7.39(1H, t, J=9.0Hz), |
| | | | | | | 7.57(1H, d, J=9.0Hz), 7.77(1H, td, J=9.0, 1.2Hz), 8.06 |
| | | | | | | (1H, dd, J=9.0, 1.2Hz) |
| | | | | | | Mass: 406(M⁺ + H) |
| (31) | H | H | H | H | CN | ¹H NMR(200MHz, DMSO-d₆, δ): 1.7-2.0(2H, m), |
| | | | | | | 2.3-2.7(8H, m), 3.3-3.5(4H, m), 6.98(2H, d, J=8Hz), |
| | | | | | | 7.39(1H, t, J=9.0Hz), 7.56(2H, d, J=8Hz), 7.57(1H, d, |
| | | | | | | J=9.0Hz), 7.80(1H, td, J=9.0, 1.2Hz), 8.06(1H, dd, |
| | | | | | | J=9.0, 1.2Hz) |
| | | | | | | Mass: 374(M⁺ + H) |
| (32) | H | H | H | H | COOH | ¹H NMR(200MHz, DMSO-d₆, δ): 1.7-2.0(2H, m), |
| | | | | | | 2.3-2.7(8H, m), 3.3-3.5(4H, m), 6.90(2H, d, J=8Hz), |
| | | | | | | 7.35(1H, t, J=9.0Hz), 7.57(1H, d, J=9.0Hz), 7.71(2H, |
| | | | | | | d, J=8Hz), 7.80(1H, td, J=9.0, 1.2Hz), 8.06(1H, dd, |
| | | | | | | J=9.0, 1.2Hz) |
| | | | | | | Mass: 393(M⁺ + H) |
| (33) | H | H | H | H | OPh | ¹H NMR(200MHz, DMSO-d₆, δ): 1.7-2.0(2H, m), |
| | | | | | | 2.3-2.7(8H, m), 3.0-3.3(4H, m), 6.8-7.0(7H, m), 7.2-7.5 |
| | | | | | | (3H, m), 7.60(1H, d, J=8Hz), 7.59(1H, t, J=8Hz), 8.06 |
| | | | | | | (1H, dJ=8.0Hz) |
| | | | | | | Mass: 441(M⁺ + H) |
| (34) | H | H | H | H | Ac | ¹H NMR(200MHz, DMSO-d₆, δ): 1.7-2.0(2H, m), |
| | | | | | | 2.3-2.7(8H, m), 3.3-3.5(4H, m), 6.93(2H, d, J=8Hz), |
| | | | | | | 7.42(1H, t, J=9.0Hz), 7.58(1H, d, J=9.0Hz), 7.77(2H, |
| | | | | | | d, J=8Hz), 7.80(1H, td, J=9.0, 1.2Hz), 8.06(1H, dd, |
| | | | | | | J=9.0, 1.2Hz) |
| | | | | | | Mass: 391(M⁺ + H) |
| (35) | H | H | H | H | Ph | ¹H NMR(200MHz, DMSO-d₆, δ): 1.7-2.0(2H, m), |
| | | | | | | 2.3-2.7(8H, m), 3.3-3.5(4H, m), 6.9-8.1(13H, m) |
| | | | | | | Mass: 391(M⁺ + H) |
| (36) | H | H | H | H | Me | ¹H NMR(200MHz, DMSO-d₆, δ): 1.7-2.0(2H, m), 2.1 |
| | | | | | | (3H, s), 2.3-2.7(8H, m), 3.3-3.5(4H, m), 6.88(2H, d, |
| | | | | | | J=8.0Hz), 6.81(2H, d, J=8Hz), 7.39(1H, t, J=9.0Hz), |
| | | | | | | 7.57(1H, d, J=9.0Hz), 7.75(1H, td, J=9.0, 1.2Hz), 8.06 |
| | | | | | | (1H, dd, J=9.0, 1.2Hz) |
| | | | | | | Mass: 363(M⁺ + H) |
| (37) | H | H | H | H | CF₃ | ¹H NMR(200MHz, DMSO-d₆, δ): 1.7-2.0(2H, m), |
| | | | | | | 2.3-2.7(8H, m), 3.3-3.5(4H, m), 6.8-8.2(8H, m) |
| | | | | | | Mass: 417(M⁺ + H) |

### EXAMPLE 15

A mixture of 8-chloro-2-[3-(4-phenyl-3,6-dihydro-1 (2H)-pyridinyl)propyl]-4(3H)-quinazolinone (50 mg), 1-methylpiperazine (19.8 mg), palladium (II) acetate (2.96 mg), 2-(di-t-butylphosphino)biphenyl (7.86 mg), sodium t-butoxide (23 mg) in toluene (0.4 ml and tetrahydrofuran (0.2 ml) was stirred at 80° C. under nitrogen atmosphere overnight. The mixture was cooled, diluted with water and extracted with dichloromethane twice. The combined extracts were dried over magnesium sulfate and concentrated. The residue was purified by preparative thin layer chromatography on silica gel using 10% methanol in dichloromethane to give the 8-(4-methyl-1-piperazinyl)-2-[3-(4-phenyl-3,6-dihydro-1(2H)-pyridinyl)propyl]-4(3H)-quinazolinone.

Mass (APCI): 444.3 (M⁺+H)

### EXAMPLE 16

The following compounds are prepared in a similar manner to that of Example 15.

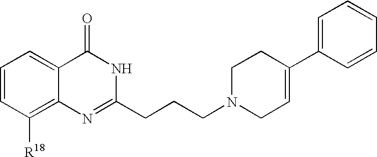

| No. | R¹⁸ | |
|---|---|---|
| (1) | 1-piperidyl | Mass (ESI): 429.3 (M⁺ + H) |
| (2) | (2R,6S)-2,6-Dimethyl- | Mass (ESI): 459.3 (M⁺ + H) |
| | 4-morpholinyl | |
| (3) | 1-pyrrolidynyl | ¹H NMR (200 MHz, DMSO-d₆, δ): 1.8-2.1 (2H, m), 2.3-2.8 |
| | | (8H, m), 3.05 (2H, br.s), 6.20 (1H, m), 7.0-7.9 (8H, m) |
| | | Mass: 415 (M⁺ + H) |
| (4) | 4-morpholinyl | ¹H NMR (200 MHz, DMSO-d₆, δ): 1.8-2.1 (2H, m), 2.1-3. |
| | | (16H, m), 3.7-3.9 (2H, m), 6.10 (1H, m), 7.0-8.0 (8H, m) |
| | | Mass: 431 (M⁺ + H) |

### EXAMPLE 17

To a suspension of 8-nitro-2-[3-(4-phenyl-3,6-dihydro-1(2H)-pyridinyl)propyl]4(3H)-quinazolinone (50 mg) in ethanol (10 ml) and water (5 ml) were added iron powder (57 mg) and ammonium chloride (5.8 mg). After stirring under reflux for 1 hour, the mixture was filtered and the filtrate was concentrated. The residue was purified by preparative thin layer chromatography using 10% methanol in dichloromethane as an eluent to give 8-Amino-2-[3-(4-phenyl-3,6-dihydro-1(2H)-pyridinyl)propyl]-4(3H)-quinazolinone as a brown powder.

¹H NMR (DMSO-d₆, δ): 1.80-2.20 (1H, m), 2.30-3.30 (10H, m), 5.58 (2H, brs), 6.13 (1H, s), 6.80-7.70 (8H, m), 12.03 (1H, brs)

Mass (ESI): 361.4 (M⁺+H)

### EXAMPLE 18

A slurry of 8-amino-2-[3-(4phenyl-3,6-dihydro-1(2H)-pyridinyl)propyl]-4(3H)-quinazolinone (40 mg), 37% aqueous formaldehyde (0.088 ml), acetic acid (0.032 ml) and sodium cyanoborohydride (70 mg) in acetonitrile (10 ml) was stirred at room temperature overnight. The reaction was quenched with aqueous sodium hydrogen carbonate and extracted with dichloromethane three times. The combined extracts were dried over magnesium sulfate and concentrated. The residue was purified by preparative thin layer chromatography using 10% methanol in dichloromethane as an eluent to give 8-dimethylamino-2-[3-(4-phenyl-3,6-dihydro-1(2H)-pyridinyl)propyl]-4(3H)-quinazolinone e (18 mg) as a yellow solid.

¹H NMR (DMSO-d₆, δ): 1.80-2.20 (2H, m), 2.30-2.90 (10H, m), 2.96 (6H, s), 6.15 (1H, s), 7.00-7.70 (8H, m), 12.15 (1H, brs)

Mass (ESI): 389.4 (M⁺+H)

### EXAMPLE 19

The following compounds are prepared in a similar manner to that of Preparation 18.

(1) 8-benzylamino-2-{3-[4-phenyl-3,6-dihydro-1(2H)pyridinyl]propyl}-4(3H)-quinazolinone

¹H NMR (200 MHz, DMSO-d₆, δ): 1.8-2.1 (2H, m), 2.1-3.0 (8H, m), 3.0-3.2 (2H, m), 4.47 (2H, d, J =6 Hz), 6.09 (1H, m), 6.56 (1H, t, J =6.2 Hz), 6.69 (1H, d, J=6.2 Hz), 7.0-7.5 (12H, m)

Mass: 451 (M⁺+H)

### EXAMPLE 20

A solution of 8-amino-2-[3-(4-phenyl-3,6-dihydro-1(2H)-pyridinyl)propyl]-4(3H)-quinazolinone (30 mg) and acetic anhydride (17 mg) in dichloromethane was stirred at room temperature overnight. The mixture was concentrated and purified by preparative thin layer chromatography (10% methanol in dichloromethane) to give N-{4-Oxo-2-[(4-phenyl-3,6-dihydro-1(2H)-pyridinyl)propyl]-3,4-dihydro-8-quinazolinyl}acetamide as a pale yellow powder.

¹H NMR (200 MHz, DMSO-d₆, δ): 1.80-2.20 (2H, m), 2.22 (3H, s), 2.30-3.00 (8H, m), 3.10 (2H, d, J=3.0 Hz), 6.10 (1H, s), 7.10-7.60 (6H, m), 7.70 (1H, dd, J=1.4 8.0 Hz), 8.57 (1H, dd, J=1.4, 8.0 Hz), 9.51 (1H, s), 12.38 (1H, brs).

Mass (ESI): 403.4 (M⁺+H)

### EXAMPLE 21

A mixture of 8-iodo-2-[3-(4-phenyl-3,6-dihydro-1(2H)-pyridinyl)propyl]-4(3H)-quinazolinone (45 mg), (trimethylsilyl)acetylene (14.1 mg), dichlorobis(triphenylphosphine)palladium (II) (6.7 mg), copper iodide (1.82 mg) and triethylamine (0.027 ml) in N,N-dimethylformamide was stirred at room temperature under nitrogen overnight. The mixture was diluted with water and extracted with dichloromethane twice. The combined extracts were washed with water twice, dried over magnesium sulfate and concentrated. The residue was purified by preparative thin layer chromatography using 10% methanol in dichloromethane as an eluent to give 2-[3-(4-Phenyl-3,6-dihydro-1(2H)-pyridinyl)propyl]-8-[(trimethylsilyl)ethynyl]-4(3H)-quinazolinone as a colorless powder (13 mg).

¹H NMR (200 MHz, CDCl₃, δ): 0.33 (9H, s), 0.70-3.30 (12H, m), 6.08 (1H, s), 7.10-8.30 (8H, m)

Mass (ESI): 441.64 (M⁺+H)

### EXAMPLE 22

A solution of 2-[3-(4-Phenyl-3,6-dihydro-1(2H)-pyridinyl)propyl]-8-[(trimethylsilyl)ethynyl]-4(3H)-quinazolinone (202 mg) in methanol was stirred at room temperature in the presence of potassium carbonate (190 mg) for 3 hours. The mixture was diluted with water and extracted with dichloromethane twice. The combined extracts were dried over magnesium sulfate and concentrated. The residue was purified by preparative thin layer chromatography on silica gel using 10% methanol in dichloromethane as an eluent to give 8-Ethynyl-2-[3-(4-phenyl-3,6-dihydro-1(2H)-pyridinyl)propyl]-4(3H)-quinazolinone the objective compound, which was converted to the corresponding hydrochloride salt (59 mg) by treatment of 4N hydrogen chloride in ethyl acetate.

¹H NMR (DMSO-d6, d): 2.10-2.40 (2H, m), 2.60-3.00 (4H, m), 3.00-4.20 (6H, m), 4.51 (1H, s), 6.22 (1H, s), 7.10-7.80 (6H, m), 7.94 (1H, dd, J=1.5, 7.9 Hz (1H, dd, J=1.5, 7.9 Hz), 10.32 (1H, brs), 12.44 (1H, brs)

Mass (APCI): 370.07 (M⁺+H)

### EXAMPLE 23

The following compounds are prepared in a similar manner to that of Example 21.

(1) 8-phenyl-2-{3-[4-phenyl-3,6-dihydro-1(2H)-pyridinyl]propyl}-4(3H)-quinazolinone

¹H NMR (200 MHz, DMSO-d₆, δ): 1.8-2.1 (2H, m), 2.1-3.0 (8H, m), 3.0-3.2 (2H, m), 6.09 (1H, m), 7.0-8.2 (13H, m)

Mass: 422 (M⁺+H)

### Example 24

Under a nitrogen atmosphere, (diethylamino)sulfur trifluoride (0.363 mL, 2.75 mmol) was added dropwise to a solution of 2-[3-(4-hydroxy-4-phenyl-1-piperidyl)propyl]-4(3H)-quinazolinone (100 mg, 0.275 mm in dichloromethane (10 mL) at −78° C. The mixture was stirred for 2 hours (to −50° C.). (Diethylamino)sulfer trifluoride (0.363 mL, 2.75 mmol) was added, and the mixture was stirred for further 2h (to 0° C.). Quenched with saturated aqueous sodium hydrogencarbonate, the organic materials were extracted with ethyl acetate. Purification over silica gel chromatography gave 2-[3-(4-fluoro-4-phenyl-1-piperidyl)propyl]-4(3H)-quinazolinone (34 mg, 33.8%).

¹H NMR (200 MHz, CDCl₃, δ): 1.9-2.1 (4H, m), 2.5-2.9 (6H, m), 2.9-3.1 (4H, m), 7.31 (1H, t, J=7.1 Hz), 7.44 (3H, t, J=7.9 Hz), 7.6-7.8 (4H, m), 8.29 (1H, d, J=7.9 Hz). MS (APCI): 365.80 (M⁺+H)

### EXAMPLE 25

2-{3-[4-phenyl-3,6-dihydro-1(2H)-pyridinyl]propyl}-4(3H)-quinazolinone (110 mg, 0.310 mmol) was suspended in a mixed solvent of chloroform (1 mL) and ethyl acetate (2 mL). To this suspension, a solution of hydrogen chloride (4M, 2.33 mL) was added, and the mixture was stirred for 1 hour. The white precipitate was collected by filtration to give 2-{3-[4-phenyl-3,6-dihydro-1(2H)-pyridinyl]propyl}4(3H)-quinazolinone hydrochloride (124 mg, 104%) as product.

¹H NMR (200 MHz, DMSO-d₆, δ): 2.29 (2H, quint., J=7.6 Hz), 2.8-2.9 (4H, m), 3.30 (2H, dd, J=8.6, 6.8 Hz), 3.5-4.2 (4H, m), 6.21 (1H, br s), 7.2-7.6 (6H, m), 7.73 (1H, d, J=7.7 Hz), 7.86 (1H, t, J=6.9 Hz), 8.13 (1H, d, J=7.9 Hz). MS (APCI): 346.13 (M⁺+H)

### EXAMPLE 26

The following compounds are prepared in a similar manner to that of Preparation 25.

(1) 8-chloro-2-{3-[4-(4-acetylphenyl)-3,6-dihydropyridin-1(2H)-yl]propyl}-4(3H)-quinazolinone hydrochloride

¹H NMR (200 MHz, DMSO-d₆, δ): 2.1-2.4 (2H, m), 2.59 (3H, s), 2.7-3.0 (4H, m), 3.2-3.5 (3H, m), 3.6-4.2 (3H, m), 6.40 (1H, br s), 7.46 (1H, t, J=7.8 Hz), 7.65 (2H, d, J=8.4 Hz), 7.9-8.0 (3H, m), 8.06 (1H, d, J=7.9 Hz), 10.65 (1H, br), 12.54 (1H, br)

Mass (APCI): 422.07 (M⁺+H)

(2) 8-chloro-2-{3-[4-phenyl-3,6-dihydropyridin-1(2H)-yl]propyl}4(3H)-quinazolinone hydrochloried

¹H NMR (200 MHz, DMSO-d₆, δ): 2.1-2.45 (2H, m), 2.65-3.05 (4H, m), 3.15-3.45 (3H, m), 3.55-3.9 (2H, m), 3.95-4.15 (1H, m), 6.20 (1H, s), 7.3-7.55 (6H, m), 7.95 (1H, dd, J=7.8, 1.4 Hz), 8.05 (1H, dd, J=7.8, 1.4 Hz)

(3) 8-chloro-2-{3-[4-[4-(tifluoromethyl)phenyl]-3,6-dihydropyridin-1(2H)-yl]propyl}-4(3H)-quinazolinone hydrochloride

¹H NMR (DMSO-d₆, δ): 2.15-2.35 (2H, m), 2.75-2.95 (4H, m), 3.25-3.45 (2H, m), 3.45-4.20 (4H, m), 6.37 (1H, s), 7.45 (1H, t, J=7.8 Hz), 7.73 (4H, s), 7.94 (1H, dd, J=7.8, 1.4 Hz), 8.05 (1H, dd, J=7.8, 1.4 Hz), 10.59 (1H, br s), 12.53 (1H, br s)

(4) 8-Chloro-2-{3-[4-(4-(hydroxymethyl)phenyl)-3,6-dihydropyridin-1(2H)-yl]-propyl}4(3H)-quinazolinone hydrochloride

¹H NMR (DMSO-d₆, δ): 2.15-2.40 (2H, m), 2.7-2.9 (4H, m), 3.6-4.2 (6H, m), 4.50 (2H, s), 5.72 (1H, s), 6.18 (1H, s), 7.32 (2H, d, J=8.3 Hz), 7.4-7.5 (3H, m), 7.94 (1H, dd, J=7.8, 1.4 Hz), 8.06 (1H, dd, J=7.8, 1.4 Hz), 10.59 (1H, br s), 12.53 (1H, br s)

### EXAMPLE 27

Under a nitrogen atmosphere, 1M boron tribromide in dichloromethane (1.99 ml) was added to a solution of

2-{3-[4-(4-methoxyphenyl)piperidin-1-yl]propyl}-4(3H)-quinazolinone (150 mg) in dichloromethane (7.5 ml) at 0° C. The mixture was stirred for 2 hours and the solvent was evaporated. The residue was diluted with aqueous sodium hydrogencarbonate and the aqueous phase was removed with decant. The crude product was triturated with a mixture of chloroform and methanol (10:1) and the resulting precipitate was collected by filtration. The precipitate was washed with chloroform-methanol and dried under reduced pressure to afford 2-{3-[4-(4-hydroxyphenyl)piperidin-1-yl]propyl}-4(3H)-quinazolinone (122 mg).

¹H NMR (200 MHz, DMSO-d₆, δ): 1.7-2.1 (4H, m), 2.1-2.3 (2H, m), 2.6-3.3 (9H, m), 6.72 (2H, d, J=8.5 Hz), 6.90 (2H, d, J=8.5 Hz), 7.51 (1H, dt, J=8.1, 1.1 Hz), 7.63 (1H, d, J=8.0 Hz), 7.82 (1H, dt, J=8.4, 1.5 Hz), 8.11 (1H, dd, J=7.9, 1.1 Hz)

Mass: 361.80(M⁺)

### EXAMPLE 28

The following compounds are prepared in a similar manner to that of Example 27.

(1) 2-{3-[4-(4-hydroxyphenyl)-3,6-dihydropyridin-1(2H)-yl]propyl}-4(3H)-quinazolinone

¹H NMR (DMSO-d₆, δ): 2.1-2.4 (2H, m), 2.65-2.95 (4H1, m), 3.2-3.5 (3H, m), 3.6-4.2 (3H, m), 6.03 (1H, s), 6.77 (2H, d, J=8.7 Hz), 7.32 (2H, d, J=8.7 Hz), 7.56 (1H, t, J=7.3 Hz), 7.67 (1H, d, J=8.1 Hz), 7.85 (1H, t, J=7.4 Hz), 8.14 (1H, dd, J=7.8, 1.2 Hz)

Mass: 362.3 (M⁺+H)

### EXAMPLE 29

Under a nitrogen atmosphere, dimethylsulfoxide (0.093 ml) in dichloromethane was added to a stirred solution of oxalylchloride (0.06 ml) in dichloromethane (10 ml) at −78° C. The mixture was stirred for 1 hour. To this solution was added a solution of 2-{3-[4-(4-hydroxymethyl)phenyl]-3,6-dihydropyridin-1(2H)-yl}propyl}-4(3H)-quinazolinone (130 mg) in a mixture of dichloromethane (1.5 ml) and dimethylsulfoxide (0.5 ml) at −70° C. The mixture was stirred for 30 minutes and to this solution was added triethyl amine (0.25 ml) at the same temperature. The whole mixture was gradually warmed to −20° C. and the reaction was quenched with water. The aqueous layer was separated and the organic layer was washed with brine, dried over magnesium sulfate. After evaporation of the solvent, the residue was purified by preparative TLC eluting with chloroform-methanol to afford 2-{3-[4-(4-formylphenyl)-3,6-dihydropyridin-1(2H)-yl]propyl}-4(3H)-quinazolinone (47 mg).

¹H NMR (200 MHz, DMSO-d₆, δ): 1.85-2.1 (2H, m), 2.4-2.8 (10H, m), 3.12 (2H, d, J=2.8 Hz), 6.35 (1H, s), 7.42 (1H, t, J=6.9 Hz), 7.5-7.65 (3H, m), 7.7-7.8 (1H, m), 7.86 (2H, d, J=8.3 Hz), 8.04 (1H, dd, J=7.9, 1.3 Hz), 9.97 (1H, s), 12.21 (1H, br s)

Mass: 374.0 (M⁺)

### EXAMPLE 30

3-Chloro-2-({4-[4-(4-cyanophenyl)-3,6-dihydro-1(2H)-pyridinyl]butanoyl}amino) benzamide (152 mg, 0.359 mmol) was dissolved in a mixed solvent of dioxane (2 mL) and methanol (3 mL). An aqueous solution of sodium hydroxide (1 M, 1.08 mL) was added to the solution at room temperature, and the mixture was stirred at that temperature for 1 hour. The organic materials were extracted with chloroform, and the organic layer was washed with water and dried over sodium sulfate. The crude product was suspended in a mixed solvent of chloroform (1 mL) and ethyl acetate (2 mL). To this suspension, a solution of hydrogen chloride (4M, 2.0 mL) was added, and the mixture was stirred for 1 hour. The white precipitate was collected by filtration to give 8-chloro-2-{3-[4-(4-cyanophenyl)-3,6-dihydropyridin-1(2H)-yl]propyl}-4(3)-quinazolinone (140 mg, 88.3%) as product.

¹H NMR (200 MHz, DMSO-d₆, δ): 2.1-2.3 (2H, m), 2.7-2.9 (4H, m), 3.2-3.4 (3H, m), 3.7-4.0 (2H, m), 4.0-4.2 (1H, m), 6.44 (1H, br s), 7.46 (1H, t, J=7.9 Hz), 7.70 (2H, d, J=8.5 Hz), 7.87 (2H, d, J=8.4 Hz), 7.95 (1H, d, J=7.8 Hz), 8.06 (1H, d, J=7.9 Hz), 10.51 (1H, br), 12.53 (1H, br)

Mass (APCI): 405.07 (M⁺+H)

### EXAMPLE 31

The following compounds are prepared in a similar manner to that of Example 9. If necessary, the starting compounds of them were prepared in similar manners of Preparation 17 and Preparation 20.

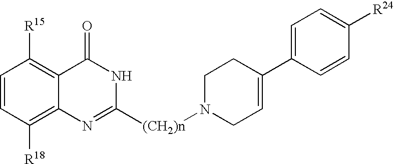

| No. | R¹⁵ | R¹⁸ | R²⁴ | n | |
|---|---|---|---|---|---|
| (1) | Cl | H | CN | 1 | ¹H NMR (200 MHz, CDCl₃, δ): 2.6-2.8 (2H, m), 2.8-3.0 (2H, m), |
| | | | | | 3.3-3.5 (2H, m), 3.66 (2H, s), 6.18 (1H, m), 7.3-7.8 (7H, m) |
| | | | | | Mass: 377 (M⁺ + H) |
| (2) | Cl | H | H | 2 | ¹H NMR (200 MHz, DMSO-d₆, δ): 2.7-3.1 (4H, m), 3.2-3.4 (2H, |
| | | | | | m), 6.39 (1H, m), 7.2-7.9 (8H, m) |
| | | | | | Mass: 366 (M⁺ + H) |
| (3) | Cl | H | CN | 2 | ¹H NMR (200 MHz, DMSO-d₆, δ): 2.7-3.1 (4H, m), 3.2-3.4 (2H, |
| | | | | | m), 6.39 (1H, m), 7.2-7.8 (7H, m) |
| | | | | | Mass: 391 (M⁺ + H) |
| (4) | Cl | H | OMe | 2 | ¹H NMR (200 MHz, DMSO-d₆, δ): 2.2-2.8 (8H, m), 3.2-3.4 (2H, |
| | | | | | m), 3.82 (3H, s), 6.03 (1H, m), 6.88 (1H, d J=8.6 Hz), 7.2-7.8 (6H, |
| | | | | | m) |
| | | | | | Mass: 396 (M⁺ + H) |
| (5) | H | Me | OMe | 2 | 1H NMR (200 MHz, DMSO-d₆, δ): 2.3-2.5 (2H, m), 2.52 (3H, s), |
| | | | | | 2.6-2.9 (6H, m), 3.74 (3H, s), 6.04 (1H, m), 6.88 (2H, d, J=8 Hz), |
| | | | | | 7.2-7.4 (3H, m), 7.62 (1H, d, J=8 Hz), 7.90 (1H, d, J=8 Hz) |
| | | | | | Mass: 376 (M⁺ + H) |
| (6) | H | Me | CN | 2 | ¹H NMR (200 MHz, DMSO-d₆, δ): 2.4-2.5 (2H, m), 2.52 (3H, s), |
| | | | | | 2.6-2.9 (6H, m), 6.40 (1H, m), 7.31 (1H, t, J=8 hz), 7.6-7.8 (5H, |
| | | | | | m), 7.90 (1H, d, J=8 Hz) |
| | | | | | Mass: 371 (M⁺ + H) |
| (7) | H | Me | CF₃ | 2 | ¹H NMR (200 MHz, DMSO-d₆, δ): 2.4-2.5 (2H, m), 2.52 (3H, s), |
| | | | | | 2.6-2.9 (6H, m), 6.35 (1H, m), 7.33 (1H, t, J=8 Hz), 7.6-7.8 (5H, |
| | | | | | m), 7.91 (1H, d, J=8 Hz) |
| | | | | | Mass: 414 (M⁺+ H) |
| (8) | H | H | H | 2 | ¹H NMR (200 MHz, CDCl₃, δ): 2.72 (2H, br), 2.9-3.0 (6H, m), 3.38 |
| | | | | | (2H, q, J=3.1 Hz), 6.10 (1H, br s), 7.3-7.5 (6H, m), 7.62 (1H, d, |
| | | | | | J=7.3 Hz), 7.72 (1H, t, J=7.6 Hz), 8.25 (1H, d, J=6.5 Hz). |
| | | | | | Mass (APCI): 331.67 (M⁺+ H) |
| (9) | H | H | H | 4 | ¹H NMR (200 MHz, CDCl₃, δ): 1.6-1.9 (2H, m), 1.95 (2H, quint., |
| | | | | | J=7.3 Hz), 2.5-2.7 (4H, m), 2.7-2.9 (4H, m), 3.22 (2H, q, J=3.1 Hz), |
| | | | | | 6.06 (1H, br s), 7.2-7.5 (6H, m), 7.67 (1H, d, J=6.8 Hz), 7.75 (1H, t, |
| | | | | | J=6.7 Hz), 8.26 (1H, d, J=6.6 Hz). |
| | | | | | Mass (APCI): 360.20 (M⁺ + H) |

### EXAMPLE 32

The following compounds are prepared in a similar manner to that of Example 9. If necessary, the starting compounds of them were prepared in similar manners of Preparation 17, Preparation 20 and preparation 23-(2)

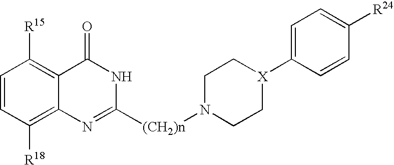

| No | R¹⁵ | R¹⁸ | R²⁴ | n | X | |
|---|---|---|---|---|---|---|
| (1) | H | Me | Cl | 2 | N | ¹H NMR (200 MHz, DMSO-d₆, δ): 2.51 (7H, m), 2.6-2.8 |
| | | | | | | (4H, m), 2.8-3.0(4H, m), 3.1-3.3 (4H, m), 6.92 (2H, d,J= |
| | | | | | | 8 Hz), 7.21 (2H, d, J=8 Hz), 7.31 (1H, t, J=8 Hz), 7.61 (1H, d, J= |
| | | | | | | 8 Hz), 7.91 (1H, d, J=8 Hz) |
| | | | | | | Mass: 383 (M⁺ + H) |
| (2) | Cl | H | Ph | 2 | N | ¹H NMR (200 MHz, DMSO-d₆, δ): 2.6-3.0 (8H, m), 3.1-3.3 |
| | | | | | | (4H, m), 7.0-7.8 (12H, m) |
| | | | | | | Mass: 445 (M⁺ + H) |
| (3) | H | Me | CN | 2 | N | ¹H NMR (200 MHz, DMSO-d₆, δ): 2.4-2.7 (7H, m), 2.6-2.8 |
| | | | | | | (4H, m), 3.2-3.3 (4H, m), 7.02 (2H, d, J=8 Hz), 7.33 (1H, t, |
| | | | | | | J=8 Hz), 7.56 (2H, d, J=8 Hz), 7.63 (1H, d, J=8 Hz), 7.91 |
| | | | | | | (1H, d, 1 8Hz) |
| | | | | | | Mass: 374 (M⁺ + H) |
| (4) | H | Cl | CN | 2 | N | ¹H NMR (200 MHz, DMSO-d₆, δ): 2.5-2.7 (4H, m), 2.7-2.9 |
| | | | | | | (4H, m), 3.1-3.3 (4H, m), 6.93 (2H, d, J=8 Hz), 7.22 (2H, d, |
| | | | | | | J=8 Hz), 7.36 (1H, t, J=8 Hz), 7.87 (1H, d, J=8 Hz), 8.01 |
| | | | | | | (1H, d, J=8 Hz) |
| | | | | | | Mass: 404 (M⁺ + H) |
| (5) | H | H | Bzl | 2 | CH | ¹H NMR (CDCl₃, δ): 1.3-1.9 (5H, m), 2.07 (2H, t, J=11.5 Hz), |
| | | | | | | 2.60 (2H, d, J=6.3 Hz), 2.7-2.9 (4H, m), 3.08 (2H, d, |
| | | | | | | J=11.9 Hz), 7.1-7.4 (5H, m), 7.43 (1H, t, J=7.4 Hz), 7.61 |
| | | | | | | (1H, d, J=7.1 Hz), 7.72 (1H, t, J=6.9 Hz), 8.27 (1H, d, J=6.5 Hz). |
| | | | | | | Mass (API-ES): 348.3 (M⁺ + H) |
| (6) | H | H | Bzl | 2 | N | ¹H NMR (200 MHz, CDCl₃, δ): 2.65 (8H, br), 2.8-2.9 (4H, |
| | | | | | | m), 3.57 (2H, s), 7.2-7.4 (5H, m), 7.43 (1H, t, J=7.4 Hz), |
| | | | | | | 7.61 (1H, d, J=7.2 Hz), 7.72 (1H, t, J=7.6 Hz), 8.27 (1H, d, |
| | | | | | | J=7.9 Hz) |
| | | | | | | Mass (API-ES): 349.4 (M⁺ + H) |

### EXAMPLE 33

The following compounds are prepared in a similar manner to that of Example 9. If necessary, the starting compounds of them were prepared in similar manners of Preparation 17 and Preparation 20.

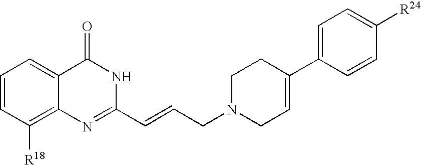

| No. | R¹⁸ | R²⁴ | |
|---|---|---|---|
| (1) | Cl | F | ¹H NMR (200 MHz, CDCl₃, δ): 2.5-2.7 (2H, m), 2.7-2.9 (2H, m), 3.01 (2H, |
| | | | d, J=3.0 Hz), 3.46 (2H, dd, J=6.0, 1.2 Hz), 6.02 (1H, , m), 6.30 (1H, d, J= |
| | | | 11.6 Hz), 7.0-7.4 (6H, m), 7.81 (1H, dd, J=8, 1.2 Hz), 8.20 (1H, dd, J=8, |
| | | | 1.2 Hz) |
| | | | Mass: 396 (M⁺ + H) |
| (2) | Cl | Cl | ¹H NMR (200 MHz, CDCl₃, δ): 2.5-2.7 (2H, m), 2.7-2.9 (2H, m), 3.2-3.3 |
| | | | (2H, m), 3.4-3.6 (2H, m), 6.10 (1H, , m), 6.55 (1H, d, J=11.6 Hz), 7.0-7.4 |
| | | | (6H, m), 7.81 (1H, dd, J=8, 1.2 Hz), 8.20 (1H, dd, J=8, 1.2 Hz) |
| | | | Mass: 413 (M⁺ + H) |
| (3) | Cl | CF₃ | ¹H NMR (200 MHz, CDCl₃, δ): 2.5-2.7 (2H, m), 2.84 (2H, t, J=5.6 Hz), |
| | | | 3.30 (2H, d, J=3.2 Hz), 3.4-3.5 (2H, m), 6.10 (1H, , m), 6.61 (1H, d, J= |
| | | | 11.6 Hz), 7.0-7.4 (6H, m), 7.83 (1H, dd, J=8, 1.2 Hz), 8.19 (1H, dd, J=8, |
| | | | 1.2 Hz) |
| | | | Mass: 445 (M⁺ + H) |
| (4) | Cl | OMe | ¹H NMR (200 MHz, CDCl₃, δ): 2.5-2.7 (2H, m), 2.82 (2H, t, J=5.4 Hz), |
| | | | 3.30 (2H, d, J=3.2 Hz), 3.4-3.5 (2H, m), 3.81 (3H, s), 6.00 (1H, m), 6.84 |
| | | | (1H, d, J=11.6 Hz), 6.8-7.4 (6H, m), 7.80 (1H, dd, J=8, 1.2 Hz), 8.20 (1H, |
| | | | dd, J=8, 1.2 Hz) |
| | | | Mass: 408 (M⁺ + H) |
| (5) | Me | OMe | ¹H NMR (200 MHz, CDCl₃, δ): 2.5-2.7 (5H, m), 2.84 (2H, t, J=5.4 Hz), |
| | | | 3.30 (2H, d, J=3.2 Hz), 3.4-3.5 (2H, m), 3.81 (3H, s), 6.01 (1H, m), 6.58 |
| | | | (1H, d, J=11.6 Hz), 6.8-7.4 (6H, m), 7.58 (1H, dd, J=8, 1.2 Hz), 8.13 (1H, |
| | | | dd, J=11.6 Hz), 7.0-7.4 (6H, m), 7.59 (1H, dd, J=8, 1.2 Hz), 8.13 (1H, |
| | | | Mass: 388 (M⁺ + H) |
| (6) | Me | Me | ¹H NMR (200 MHz, CDCl₃, δ): 2.23 (3H, s), 2.5-2.7 (5H, m), 2.84 (2H, t, J= |
| | | | 5.4 Hz), 3.30 (2H, d, J=3.2 Hz), 3.4-3.5 (2H, m), 6.06 (1H, m), 6.62 (1H, d, |
| | | | J=11.6 Hz), 7.0-7.4 (6H, m), 7.59 (1H, dd, J=8, 1.2 Hz), 8.10 (1H, dd, J=8, |
| | | | 1.2 Hz) |
| | | | Mass: 372 (M⁺ + H) |
| (7) | Me | CF₃ | ¹H NMR (200 MHz, CDCl₃, δ): 2.2-2.4 (8H, m), 2.81 (2H, t, J=5.4 Hz), |
| | | | 3.22 (2H, d, J=3.2 Hz), 3.4-3.5 (2H, m), 6.20 (1H, m), 6.78 (1H, d, J= |
| | | | 11.6 Hz), 7.0-7.6 (7H, m), 8.12 (1H, dd, J=8, 1.2 Hz) |
| | | | Mass: 426 (M⁺ + H) |
| (8) | Me | F | ¹H NMR (200 MHz, CDCl₃, δ): 2.2-2.4 (8H, m), 2.81 (2H, t, J=5.4 Hz), |
| | | | 3.22 (2H, d, J=3.2 Hz), 3.4-3.5 (2H, m), 6.20 (1H, m), 6.78 (1H, d, J= |
| | | | 11.6 Hz), 7.0-7.6 (7H, m), 8.12 (1H, dd, J=8, 1.2 Hz) |
| | | | Mass: 376 (M⁺ + H) |
| (9) | Me | Cl | ¹H NMR (200 MHz, CDCl₃, δ): 2.2-2.4 (8H, m), 2.81 (2H, t, J=5.4 Hz), |
| | | | 3.22 (2H, d, J=3.2 Hz), 3.4-3.5 (2H, m), 6.23 (1H, m), 6.55 (1H, d, J= |
| | | | 11.6 Hz), 7.0-7.6 (711, m), 8.00 (1H, dd, J=8, 1.2 Hz) |
| | | | Mass: 392 (M⁺ + H) |
| (10) | H | CF₃ | ¹H NMR (200 MHz, CDCl₃, δ): 2.5-2.6 (2H, m), 2.86 (2H, t, J=5.4 Hz), |
| | | | 3.34 (2H, d, J=3.2 Hz), 3.4-3.5 (2H, m), 6.20 (1H, m), 6.59 (1H, d, J= |
| | | | 11.6 Hz), 7.0-7.8 (8H, m), 8.26 (1H, d, J=7.8 Hz) |
| | | | Mass: 412 (M⁺ + H) |
| (11) | H | F | ¹H NMR (200 MHz, CDCl₃, δ): 2.5-2.6 (2H, m), 2.86 (2H, t, J=5.4 Hz), |
| | | | 3.32 (2H, d, J=3.2 Hz), 3.4-3.5 (2H, m), 6.03 (1H, m), 6.59 (1H, d, J= |
| | | | 11.6 Hz), 7.0-7.8 (8H, m), 8.32 (1H, d, J=7.8 Hz) |
| | | | Mass: 362 (M⁺ + H) |
| (12) | H | Cl | ¹H NMR (200 MHz, CDCl₃, δ): 2.5-2.6 (2H, m), 2.86 (2H, t, J=5.4 Hz), |
| | | | 3.32 (2H, d, J=3.2 Hz), 3.4-3.5 (2H, m), 3.77 (3H, s), 6.03 (1H, m), 6.59 |
| | | | (1H, d, J=11.6 Hz), 6.8-7.8 (8H, m), 8.29(1H, d, J=7.8 Hz) |
| | | | Mass: 374 (M⁺ + H) |
| (13) | H | Cl | ¹H NMR (200 MHz, CDCl₃, δ): 2.5-2.6 (2H, m), 2.86 (2H, t, J=5.4 Hz), |
| | | | 3.32 (2H, d, J=3.2 Hz), 3.4-3.5 (2H, m), 6.05 (1H, m), 6.51 (1H, d, J= |
| | | | 11.6 Hz), 6.8-7.8 (8H, m), 8.22 (1H, d, J=7.8 Hz) |
| | | | Mass: 378 (M⁺ + H) |
| (14) | H | H | ¹H NMR (200 MHz, CDCl₃, δ): 2.5-2.6 (2H, m), 2.86 (2H, t, J=5.4 Hz), |
| | | | 3.32 (2H, d, J=3.2 Hz), 3.4-3.5 (2H, m), 6.10 (1H, m), 6.58 (1H, d, J= |
| | | | 11.6 Hz), 7.0-7.8 (9H, m), 8.27 (1H, d, J=7.8 Hz) |
| | | | Mass: 344 (M⁺ + H) |

### EXAMPLE 34

The following compounds are prepared in a similar manner to that of Example 9. If necessary, the starting compounds of them were prepared in similar manners of Preparation 17 and Preparation 20.

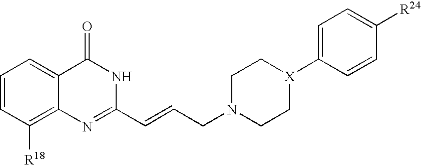

| No. | X | R¹⁸ | R²⁴ | |
|---|---|---|---|---|
| (1) | CH | Cl | Cl | ¹H NMR (200 MHz, CDl₃, δ): 1.8-2.6 (7H, m), 3.0-3.3 (2H, m), |
| | | | | 3.3-3.5 (2H, m), 6.62 (1H, d, J=12 Hz), 7.0-7.5 (6H, m), 7.82 (1H, |
| | | | | dd, J=8.0, 1.4 Hz), 8.20 (1H, dd, J=8.0, 1.4 Hz) |
| | | | | Mass: 415 (M⁺ + H) |
| (2) | CH | Cl | CF₃ | ¹H NMR (200 MHz, CDCl₃, δ): 1.8-2.8 (7H, m), 3.1-3.3 (2H, m), |
| | | | | 3.3-3.5 (2H, m), 6.62 (1H, d, J=12 Hz), 7.0-7.6 (6H, m), 7.84 (1H, |
| | | | | dd, J=8.0, 1.4 Hz), 8.20 (1H, dd, J=8.0, 1.4 Hz) |
| | | | | Mass: 448 (M⁺ + H) |
| (3) | CH | Cl | OMe | ¹H NMR (200 MHz, CDCl₃, δ): 1.8-2.8 (7H, m), 3.1-3.3 (2H, m), |
| | | | | 3.3-3.5 (2H, m), 3.79 (3H, s), 6.59 (1H, d, J=12 Hz), 6.8-7.4 (6H, |
| | | | | m), 7.84 (1H, dd, J=8.0, 1.4 Hz), 8.20 (1H, dd, J=8.0, 1.4 Hz) |
| | | | | Mass: 410 (M⁺ + H) |
| (4) | CH | Me | CF₃ | ¹H NMR (200 MHz, CDCl₃, δ): 1.8-2.8 (7H, m), 2.64 (3H, s), |
| | | | | 3.1-3.3 (2H, m), 3.3-3.5 (2H, m), 6.54(1H, d, J=12 Hz), 7.0-7.4 |
| | | | | (7H, m), 8.15 (1H, dd, J=8.0, 1.4 Hz) |
| | | | | Mass: 428 (M⁺ + H) |
| (5) | CH | Me | OMe | ¹H NMR (200 MHz, CDCl₃, δ): 1.8-2.8 (7H, m), 2.64 (3H, s), |
| | | | | 3.1-3.3 (2H, m), 3.3-3.5 (2H, m), 3.79 (3H, s), 6.51 (1H, d, J= |
| | | | | 12 Hz), 6.8-7.6(7H, m), 8.16(1H, dd, J=8.0, 1.4 Hz) |
| | | | | Mass: 390 (M⁺ + H) |
| (6) | CH | Me | Me | ¹H NMR (200 MHz, CDCl₃, δ): 1.8-2.8 (7H, m), 2.32 (3H, s), 2.64 |
| | | | | (3H, s), 3.1-3.3 (2H, m), 3.3-3.5 (2H, m), 6.51 (1H, d, J=12 Hz), |
| | | | | 6.8-7.6 (7H, m), 8.16 (1H, dd, J=8.0, 1.4 Hz) |
| | | | | Mass: 374 (M⁺ + H) |
| (7) | CH | Me | Cl | ¹H NMR (200 MHz, CDCl₃, δ): 1.8-2.8 (7H, m), 2.64 (3H, s), |
| | | | | 3.1-3.3 (2H, m), 3.3-3.5 (2H, m), 6.51 (1H, d, J=12 Hz), 6.8-7.6 |
| | | | | (7H, m), 8.16(1H, dd, J=8.0, 1.4 Hz) |
| | | | | Mass: 394 (M⁺ + H) |
| (8) | CH | Me | F | ¹H NMR (200 MHz, CDCl₃, δ): 1.8-2.8 (7H, m), 2.64 (3H, s), |
| | | | | 3.1-3.3 (2H, m), 3.3-3.5 (2H, m), 6.51 (1H, d, J=12 Hz), 6.8-7.6 |
| | | | | (7H, m), 8.20 (1H, dd, J=8.0, 1.4 Hz) |
| | | | | Mass: 367 (M⁺ + H) |
| (9) | N | Me | F | ¹H NMR (200 MHz, CDCl₃, δ): 2.63 (3H, s), 2.7-2.9 (2H, m), |
| | | | | 3.1-3.3 (2H, m), 3.4-3.6 (2H, m), 6.58 (1H, d, J=16.2 Hz), 6.8-7.6 |
| | | | | (6H, m), 7.60 (1H, d, J=7.0 Hz), 8.15 (1H, dd, J=7.0, 1.4 Hz) |
| | | | | Mass: 378 (M⁺ + H) |
| (10) | N | Me | CN | ¹H NMR (200 MHz, CDCl₃, δ): 2.3-2.8 (7H, m), 3.2-3.5 (6H, m), |
| | | | | 6.45 (1H, d, J=15 Hz), 6.8-7.8 (7H, m), 7.91 (1H, d, J=8 Hz) |
| | | | | Mass: 386 (M⁺ + H) |
| (11) | N | Me | Cl | ¹H NMR (200 MHz, CDCl₃, δ): 2.3-2.8 (7H, m), 3.2-3.5 (6H, m), |
| | | | | 6.45 (1H, d, J=15 Hz), 6.8-7.8 (7H, m), 7.91 (1H, d, J=8 Hz) |
| | | | | Mass: 395 (M⁺ + H) |
| (12) | N | Cl | Cl | ¹H NMR (200 MHz, CDCl₃, δ): 2.5-2.7 (4H, m), 3.2-3.4 (4H, m), |
| | | | | 3.4-3.6 (2H, m), 6.62 (1H, d, J=16 Hz), 6.81 (2H, d, J=8 Hz), |
| | | | | 7.1-7.4(4H, m), 7.84 (1H, dd, J=8, 1.2 Hz), 8.20 (1H, dd, J= |
| | | | | 8, 1.2 Hz) |
| | | | | Mass: 416 (M⁺ + H) |
| (13) | N | Cl | F | ¹H NMR (200 MHz, CDCl₃, δ): 2.5-2.7 (4H, m), 3.2-3.4 (4H, m), |
| | | | | 3.4-3.6 (2H, m), 6.64(1H, d, J=16 Hz), 6.7-7.4 (6H, m), 7.84 (1H, |
| | | | | dd, J=8, 1.2 Hz), 8.21 (1H, dd, J=8, 1.2 Hz) |
| | | | | Mass: 399 (M⁺ + H) |
| (14) | N | Cl | CN | ¹H NMR (200 MHz, CDCl₃): d ¹H NMR (200 MHz, CDCl₃, |
| | | | | δ): 2.5-2.7 (4H, m), 3.2-3.4 (4H, m), 3.4-3.6 (2H, m), 6.62 (1H, d, J= |
| | | | | 16 Hz), 6.7-7.4 (6H, m), 7.84 (1H, dd, J=8, 1.2 Hz), 8.20 (1H, dd, J= |
| | | | | 8, 1.2 Hz) |
| | | | | Mass: 406 (M⁺ + H) |
| (15) | N | H | Cl | ¹H NMR (200 MHz, CDCl₃, δ): 2.5-2.7(4H, m), 3.2-3.4(4H, m), |
| | | | | 3.4-3.6 (2H, m), 6.52 (1H, d, J=16 Hz), 6.7-7.4 (8H, m), 8.28 (1H, |
| | | | | dd, J=8, 1.2 Hz) |
| | | | | Mass: 381. (M⁺ + H) |

### EXAMPLE 35

The following compounds are prepared in a similar manner to that of Example 9. If necessary, the starting compounds of them were prepared in similar manners of Preparation 17 and Preparation 20

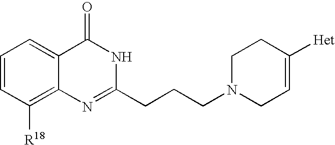

| No. | R¹⁸ | Het | |
|---|---|---|---|
| (1) | H | 1,3-thiazol-2-yl | ¹H NMR (200 MHz, CDCl₃, δ): 2.06 (2H, quint., J=6.4 Hz), |
| | | | 2.67 (2H, t, J=6.1 Hz), 2.8-3.0 (6H, m), 3.34 (2H, d, J=3.3 |
| | | | Hz), 6.62 (1H, t, J=3.7 Hz), 7.23 (1H, d, J=3.3 Hz), 7.41 (1H, |
| | | | t, J=7.3 Hz), 7.6-7.7 (2H, m), 7.77 (1H, d, J=3.3 Hz), 8.22 |
| | | | (1H, d, J=3.9 Hz), 12.22 (1H, br). |
| | | | Mass (APCI): 352.93 (M⁺ + H) |
| (2) | H | 1-methyl-1H- | ¹H NMR (200 MHz, CDCl₃, δ): 2.05 (2H, quint., J=6.0 Hz), |
| | | imidazol-2-yl | 2.69 (2H, t, J=5.9 Hz), 2.8-3.0 (6H, m), 3.32 (2H, d, J=3.2 |
| | | | Hz), 3.79 (3H, s), 5.97 (1H, t, J=3.4 Hz), 6.86 (1H, d, J=1.1 |
| | | | Hz), 7.02 (1H, d, J=1.1 Hz), 7.41 (1H, t, J=8.1 Hz), 7.63 (1H, |
| | | | d, J=6.9 Hz), 7.71 (1H, t, J=8.2 Hz), 8.20 (1H, d, J=8.0 Hz). |
| | | | Mass (APCI): 349.93 (M⁺ + H) |
| (3) | H | 1-methyl-1H- | ¹H NMR (200 MHz, CDCl₃, δ): 2.05 (2H, quint., J=5.9 Hz), |
| | | | 2.69 (4H, t, J=5.8 Hz), 2.8-3.0 (4H, m), 3.31 (2H, q, J=3.1 |
| | | | Hz), 3.97 (3H, s), 5.89 (1H, br s), 6.20 (1H, d, J=1.9 Hz), 7.42 |
| | | | (1H, t, J=7.3 Hz), 7.43 (1H, d, J=1.8 Hz), 7.63 (1H, d, J=7.0 |
| | | | Hz), 7.72 (1H, t, J=6.8 Hz), 8.23 (1H, d, J=8.0 Hz). |
| | | | (APCI): 350.00 (M⁺ + H) |
| (4) | H | 2-thienyl | ¹H NMR (200 MHz, CDCl₃, δ): 2.04 (2H, quint., J=6.3 Hz), |
| | | | 2.64 (2H, t, J=6.1 Hz), 2.8-3.0 (6H, m), 3.28 (2H, d, J=3.2 |
| | | | Hz), 6.12 (1H, br s), 6.9-7.1 (2H, m), 7.15 (1H, d, J=4.9 Hz), |
| | | | 7.42 (1H, t, J=8.1 Hz), 7.63 (1H, d, J=6.9 Hz), 7.72 (1H, t, |
| | | | J=6.7 Hz), 8.23 (1H, d, J=8.0 Hz) |
| | | | Mass (APCI): 351.87 (M⁺ + H) |
| (5) | Cl | 2-thienyl | ¹H NMR (200 MHz, CDCl₃, δ): 2.05 (2H, quint., J=6.0 Hz), |
| | | | 2.67 (2H, t, J=5.9 Hz), 2.8-3.0 (6H, m), 3.31 (2H, d, J=3.4 |
| | | | Hz), 6.12 (1H, t, J=3.5 Hz), 6.9-7.1 (2H, m), 7.15 (1H, d, |
| | | | J=4.9 Hz), 7.31 (1H, t, J=7.8 Hz), 7.78 (1H, d, J=7.7 Hz), 8.14 |
| | | | (1H, d, J=7.9 Hz). |
| | | | Mass (APCI): 385.80 (M⁺ + H) |
| (6) | H | 3-thienyl | ¹H NMR (200 MHz, CDCl₃, δ): 2.04 (2H, quint., J=5.1 Hz), |
| | | | 2.64 (2H, t, J=6.0 Hz), 2.7-3.0 (6H, m), 3.29 (2H, d, J=3.3 |
| | | | Hz), 6.11 (1H, br s), 7.1-7.3 (3H, m), 7.41 (1H, t, J=8.1 Hz), |
| | | | 7.6-7.8 (2H, m), 8.23 (1H, d, J=8.4 Hz), 12.47 (1H, br) |
| | | | Mass (APCI): 352.13 (M⁺ + H) |
| (7) | Cl | 3-thienyl | ¹H NMR (200 MHz, DMSO-d6, δ): 1.97 (2H, quint., J=7.0 |
| | | | Hz), 2.39 (2H, br), 2.4-2.5 (2H, m), 2.61 (2H, t, J=5.3 Hz), |
| | | | 2.73 (2H, t, J=7.3 Hz), 3.06 (2H, d, J=3.1 Hz), 6.01 (1H, br s), |
| | | | 6.9-7.1 (2H, m), 7.34 (1H, d, J=6.3 Hz), 7.38 (1H, t, J=7.8 |
| | | | Hz), 7.91 (1H, d, J=7.8 Hz), 7.99 (1H, d, J=7.9 Hz) |
| | | | Mass (API-ES): 386.2 (M⁺ + H) |
| (8) | H | 4-methyl-2-thienyl | ¹H NMR (200 MHz, CDCl₃, δ): 2.04 (2H, quint., J=6.3 Hz), |
| | | | 2.22 (3H, s), 2.63 (2H, t, J=6.1 Hz), 2.7-3.0 (6H,, m), 3.26 |
| | | | (2H, d, J=3.3 Hz), 6.07 (1H, t, J=3.6 Hz), 6.71 (1H, s), 6.83 |
| | | | (1H, s), 7.41 (1H, t, J=7.3 Hz), 7.5-7.8 (2H, m), 8.23 (1H, d, |
| | | | J=7.8 Hz) |
| | | | Mass (APCI): 366.00 (M⁺ + H) |
| (9) | H | 5-acetyl-2-thienyl | ¹H NMR (200 MHz, DMSO-d₆, δ): 1.94 (2H, quint., J=7.0 |
| | | | Hz), 2.3-2.7 (11H, m), 3.08 (2H, br s), 6.31 (1H, br s), 7.15 |
| | | | (1H, d, J=3.9 Hz), 7.42 (1H, t, J=7.1 Hz), 7.59 (1H, d, J=8.0 |
| | | | Hz), 7.76 (1H, t, J=7.1 Hz), 7.82 (1H, d, J=4.0 Hz), 8.04 (1H, |
| | | | d, J=7.8 Hz), 12.19 (1H, br s) |
| | | | Mass (APCI): 394.00 (M⁺ + H) |
| (10) | H | 5-chloro-2-thienyl | ¹H NMR (200 MHz, DMSO-d₆, δ): 1.93 (2H, quint., J=7.3 |
| | | | Hz), 2.3-2.7 (8H, m), 3.04 (2H, d, J=2.9 Hz), 5.98 (1H, br s), |
| | | | 6.87 (1H, d, J=3.9 Hz), 7.01 (1H, d, J=3.9 Hz), 7.43 (1H, t, |
| | | | J=7.5 Hz), 7.59 (1H, d, J=7.5 Hz), 7.76 (1H, t, J=7.1 Hz), 8.05 |
| | | | (1H, d, J=7.9 Hz), 12.20 (1H, br s) |
| | | | Mass (APCI): 385.87 (M⁺ + H) |
| (11) | H | 5-cyano-2-thienyl | ¹H NMR (200 MHz, DMSO-d₆, δ): 1.94 (2H, quint., J=7.2 |
| | | | Hz), 2.3-2.8 (8H, m), 3.09 (2H, d, J=2.9 Hz), 6.31 (1H, s), |
| | | | 7.20 (1H, d, J=3.9 Hz), 7.42 (1H, t, J=7.5 Hz), 7.58 (1H, d, |
| | | | J=7.7 Hz), 7.76 (1H, t, J=7.6 Hz), 7.86 (1H, d, J=4.0 Hz), 8.04 |
| | | | (1H, d, J=7.9 Hz), 12.19 (1H, br) |
| (12) | H | 5-methyl-2-thienyl | ¹H NMR (200 MHz, CDCl₃, δ): 2.03 (2H, quint., J=6.3 Hz), |
| | | | 2.45 (3H, s), 2.63 (2H, t, J=6.1 Hz), 2.7-3.0 (6H, m), 3.26 |
| | | | (2H, d, J=3.1 Hz), 5.97 (1H, br s), 6.62 (1H, d, J=3.5 Hz), |
| | | | 6.79 (1H, d, J=3.5 Hz), 7.41 (1H, t, J=7.3 Hz), 7.63 (1H, d, |
| | | | J=7.0 Hz), 7.71 (1H, t, J=6.8 Hz), 8.23 (1H, d, J=7.8 Hz) |
| | | | Mass (APCI): 365.93 (M⁺ + H) |
| (13) | H | 2-pyridinyl | ¹H NMR (200 MHz, CDCl₃, δ): 2.06 (2H, quint., J=6.1 Hz), |
| | | | 2.68 (2H, t, J=6.0 Hz), 2.8-3.0 (6H, m), 3.37 (2H, d, J=3.9 |
| | | | Hz), 6.69 (1H, t, J=3.4 Hz), 7.16 (1H, dd, J=7.4, 4.8 Hz), |
| | | | 7.3-7.5 (2H, m), 7.6-7.8 (3H, m), 8.22 (1H, d, J=7.8 Hz), 8.57 |
| | | | (1H, d, J=3.9 Hz) |
| | | | Mass (API-ES): 347.2 (M⁺ + H) |
| (14) | H | 3-pyridinyl | ¹H NMR (200 MHz, CDCl₃, δ): 2.06 (2H, quint., J=6.1 Hz), |
| | | | 2.68 (2H, t, J=5.9 Hz), 2.8-3.0 (6H, m), 3.32 (2H, d, J=3.2 |
| | | | Hz), 6.15 (1H, br s), 7.28 (1H, dd,, J=7.9, 4.9 Hz), 7.41 (1H, t, |
| | | | J=7.3 Hz), 7.6-7.8 (3H, m), 8.22 (1H, d, J=7.9 Hz), 8.50 (1H, |
| | | | d, J=4.8 Hz), 8.71 (1H, d, J=2.1 Hz), 12.60 (1H, br) |
| | | | Mass (APCI): 347.13 (M⁺ + H) |
| (15) | Cl | 4-pyridinyl | ¹H NMR (200 MHz, DMSO-d₆, δ): 1.9-2.1 (2H, m), 2.37 (2H, |
| | | | s), 2.45-2.8 (6H, m), 3.10 (2H, d, J=2.8 Hz), 6.15 (1H, s), |
| | | | 7.3-7.4 (3H, m), 7.90 (1H, dd, J=7.8, 1.4 Hz), 7.97 (1H, dd, |
| | | | J=7.8, 1.4 Hz), 8.3-8.4 (2H, m), 12.44 (1H, br s) |
| (16) | H | 4-pyridinyl | ¹H NMR (200 MHz, CDCl₃, δ): 2.06 (2H, quint., J=6.1 Hz), |
| | | | 2.68 (2H, t, J=6.0 Hz), 2.7-3.0 (6H, m), 3.33 (2H, d, J=3.3 |
| | | | Hz), 6.33 (1H, br s), 7.33 (2H, d, J=6.2 Hz), 7.41 (1H, t, J=7.4 |
| | | | Hz), 7.64 (1H, d, J=7.0 Hz), 7.72 (1H, t, J=7.5 Hz), 8.22 (1H, |
| | | | d, J=7.9 Hz), 8.57 (2H, d, J=6.2 Hz), 12.49 (1H, br) |
| | | | Mass (API-ES): 347.3 (M⁺ + H) |

### EXAMPLE 36

The following compounds are prepared in a similar manner to that of Example 9. If necessary, the starting compounds of them were prepared in similar manners of Preparation 17 and Preparation 20.

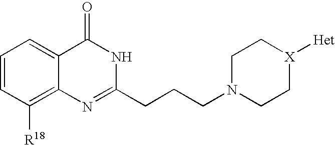

| No. | R¹⁸ | X | Het | |
|---|---|---|---|---|
| (1) | H | CH | 1-methyl-1H- | ¹H NMR (200 MHz, CDCl₃, δ): 1.8-2.0 (4H, m), 2.1-2.4 |
| | | | pyrazol-5-yl | (4H, m), 2.5-2.7 (3H, m), 2.8-3.0 (2H, m), 3.1-3.3 (2H, m), |
| | | | | 6.32 (1H, br s), 7.3-7.5 (2H, m), 7.63 (1H, d, J=6.9 Hz), |
| | | | | (1H, t, J=6.8 Hz), 8.27 (1H, d, J=7.7 Hz) |
| | | | | Mass (APCI): 361.93 (M⁺ + H) |
| (2) | H | CH | 2-thienyl | ¹H NMR (200 MHz, CDCl₃, δ): 1.9-2.4 (8H, m), 2.58 (2H, t, |
| | | | | J=5.7 Hz), 2.8-3.0 (3H, m), 3.14 (2H, br d, J=5.0 Hz), |
| | | | | 6.9-7.0 (2H, m), 7.15 (1H, d, J=6.3 Hz), 7.42 (1H, t), |
| | | | | 7.6-7.8 (2H, m), 8.27 (1H, d, J=7.8 Hz) |
| | | | | Mass (APCI-ES): 354.3 (M⁺ + H) |
| (3) | H | CH | 3-Thienyl | ¹H NMR (200 MHz, CDCl₃, δ): 1.8-2.0 (4H, m), 2.2-2.4 |
| | | | | (4H, m), 2.5-2.6 (2H, m), 2.6-2.8 (1H, m), 2.9-3.0 (2H, m), |
| | | | | 3.16 (2H, br d, J=5.4 Hz), 7.1-7.3 (3H, m), 7.42 (1H, t), |
| | | | | 7.6-7.8 (2H, m), 8.27 (1H, d, J=7.9 Hz) |
| | | | | Mass (APCI): 354.13 (M⁺ + H) |
| (4) | H | CH | 4-methyl-2- | ¹H NMR (200 MHz, CDCl₃, δ): 1.9-2.3 (11H, m), 2.56 (2H, |
| | | | thienyl | J=5.7 Hz), 2.7-3.0 (3H, m), 3.12 (2H, br d, J=7.3 Hz), |
| | | | | 6.71 (1H, s), 6.77 (1H, s), 7.42 (1H, t, J=7.4 Hz), 7.62 (1H, |
| | | | | d, J=7.1 Hz), 7.71 (1H, t, J=6.7 Hz), 8.26 (1H, d, J=8.0 Hz) |
| | | | | MS (APCI): 368.20 (M⁺ + H) |
| (5) | H | CH | 5-methyl-2- | ¹H NMR (200 MHz, CDCl₃, δ): 1.9-2.3 (8H, m), 2.45 (3H, |
| | | | thienyl | s), 2.56 (2H, t, J=5.7 Hz), 2.7-3.0 (3H, m), 3.0-3.2 (2H, m), |
| | | | | 6.60 (1H, d, J=3.3 Hz), 6.73 (1H, d, J=3.3 Hz), 7.41 (1H, t, |
| | | | | J=7.3 Hz), 7.5-7.8 (2H, m), 8.27 (1H, d, J=8.0 Hz) |
| | | | | Mass (APCI): 368.13 (M⁺ + H) |
| (6) | H | CH | 4-pyridinyl | ¹H NMR (200 MHz, CDCl₃, δ): 1.87 (2H, br d, J=11.1 Hz), |
| | | | | 1.99 (2H, quint., J=5.5 Hz), 2.1-2.4 (4H, m), 2.4-2.7 (3H, |
| | | | | m), 2.9-3.0 (2H, m), 3.23 (2H, br d, J=9.4 Hz), 7.38 (2H, d, |
| | | | | J=6.1 Hz), 7.44 (1H, t, J=8.9 Hz), 7.63 (1H, d), 7.72 (1H, t, |
| | | | | J=6.8 Hz), 8.30 (1H, d, J=8.4 Hz), 8.57 (1H, d, J=6.1 Hz) |
| | | | | Mass (APCI): 348.87 (M⁺ + H) |
| (7) | H | N | 2-pyridinyl | ¹H NMR (200 MHz, DMSO-d₆, δ): 1.7-2.0 (2H, m), 2.3-2.7 |
| | | | | (8H, m), 3.3-3.4 (4H, m), 7.40 (1H, t, J=8 Hz), 7.48 (1H, d, |
| | | | | J=8 Hz), 7.7-8.2 (4H, m), 8.26 (1H, d, J=1.2 Hz) |
| | | | | Mass: 350 (M + 1) |
| (8) | Cl | N | 2-pyridinyl | ¹H NMR (200 MHz, DMSO-d₆, δ): 1.7-2.0 (2H, m), 2.3-2.8 |
| | | | | (8H, m), 3.1-3.4 (2H, m), 6.61 (1H, m), 7.2-8.0 (6H, m), |
| | | | | 8.51 (1H, m) |
| | | | | Mass: 381 (M⁺ + H) |
| (9) | H | N | 4-pyridinyl | ¹H NMR (200 MHz, DMSO-d₆, δ): 1.7-2.0 (2H, m), 2.3-2.7 |
| | | | | (8H, m), 3.2-3.4 (4H, m), 6.76 (2H, d, J=8 Hz), 7.42 (1H, t, |
| | | | | J=8 Hz), 7.58 (1H, d, J=8 Hz), 7.72 (1H, t, J=8 Hz), |
| | | | | 8.1-8.3 (3H, m) |
| | | | | Mass: 350 (M⁺ + H) |
| (10) | CL | N | 4-pyridinyl | ¹H NMR (200 MHz, DMSO-d₆, δ): 1.7-2.0 (2H, m), 2.3-2.8 |
| | | | | (8H, m), 3.1-3.4 (2H, m), 6.41 (1H, m), 7.3-7.5 (2H, m), |
| | | | | 7.78 (1H, d, J=8 Hz), 7.91 (1H, d, J=8 Hz), 8.3-8.6 (2H, |
| | | | | m) |
| | | | | Mass: 381 (M⁺ + H) |
| (11) | H | N | 2-pyrazinyl | ¹H NMR (200 MHz, DMSO-d₆, δ): 1.7-2.0 (2H, m), 2.3-2.7 |
| | | | | (8H, m), 3.3-3.4 (4H, m), 7.40 (1H, t, J=8 Hz), 7.48 (1H, d, |
| | | | | J=8 Hz), 7.7-8.2 (3H, m), 8.26 (1H, d, J=1.2 Hz) |
| | | | | Mass: 351 (M⁺ + H) |

### EXAMPLE 37

The following compounds are prepared in a similar manner to that of Example 25.

(1) 8-Chloro-2-{3-[4-(2-thienyl)-3,6-dihydro-1(2H)-pyridinyl]propyl}-4(3H)-quinazolinone hydrochloride

¹H NMR (200 MHz, DMSO-d₆, δ): 2.1-2.4 (2H, m), 2.7-2.9 (4H, m), 3.1-3.4 (2H, m), 3.4-3.8 (3H, m), 3.9-4.1 (1H, m), 6.10 (1H, br s), 7.07 (1H, d, J=3.6 Hz), 7.20 (1H, d, J=3.6 Hz), 7.4-7.6 (2H, m), 7.95 (1H, d, J=7.8 Hz), 8.06 (1H, d, J=7.8 Hz), 10.20 (1H, br), 12.51 (1H, br s)

Mass (APCI): 385.80 (M⁺+H)

(2) 8-Chloro-2-{3-[4-(3-thienyl)-3,6-dihydro-1(2H)-pyridinyl]propyl}-4(3H)-quinazolinone hydrochloride

¹H NMR (200 MHz, DMSO-d₆, δ): 2.21 (2H, quint., J=8.2 Hz), 2.79 (4H, t, J=6.8 Hz), 3.1-3.4 (3H, m), 3.7-3.9 (2H, m), 3.9-4.1 (1H, m), 6.09 (1H, br s), 7.07 (1H, dd, J=7.0, 3.6 Hz), 7.19 (1H, d, J3.30 Hz), 7.4-7.6 (2H, m), 7.95 (1H, d, J=7.8 Hz), 8.06 (1H, d, J=7.9 Hz), 10.53 (1H, br), 12.52 (1H, br s)

Mass (APCI): 385.80 (M⁺+H)

(3) 8-Chloro-2-{3-[4-(4-pyridinyl)-3,6-dihydro-1(2H)-pyridinyl]propyl}-4(3H)-quinazolinone dihydrochloride

¹H NMR (DMSO-d₆, δ): 6.79 (1H, s), 7.45 (1H, t, J=7.9 Hz), 7.87 (2H, d, J=6.6 Hz), 7.94 (1H, dd, J=7.9,1.4 Hz), 8.06 (1H, dd, J=7.9, 1.4 Hz), 8.77 (2H, d, J=6.6 Hz), 12.52 (1H, br s)

### EXAMPLE 38

The following compounds are prepared in a similar manner to that of Example 9. If necessary, the starting compounds of them were prepared in similar manners of Preparation 17 and Preparation 20.

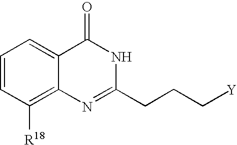

| No. | R18 | Y | |
|---|---|---|---|
| (1) | H | 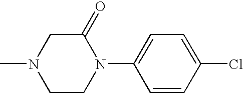 | ¹H NMR (200 MHz, CDCl₃, δ): 1.8-2.1 (2H, m), 2.4-3.0 (6H, m), 3.17 (2H, s), 3.55 (2H, t, J=35.3 Hz), 7.2-8.0 (8H, m), 12.21 (1H, brs) Mass (APCI): 419.2 (M⁺+ Na) |
| (2) | H | 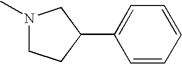 | ¹H NMR (200 MHz, CDCl₃, δ): 2.0-2.2 (3H, m), 2.4-2.7 (1H, m), 2.79 (1H, t, J=9.8 Hz), 2.8-3.0 (5H, m), 3.27 (1H, q, J=9.7 Hz), 3.48 (1H, t, J=8.8 Hz), 3.72 (1H, quint., J=8.7 Hz), 7.1-7.5 (6H, m), 7.62 (1H, d, J=6.8 Hz), 7.70 (1H, t, J=6.8 Hz), 8.21 (1H, d, J=7.9 Hz) Mass (APCI): 334.20 (M⁺ + H) |
| (3) | H | 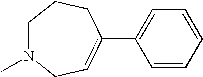 | ¹H NMR (200 MHz, CDCl₃, δ): 1.8-2.1 (4H, m), 2.73 (2H, t, J=5.9 Hz), 2.8-2.9 (4H, m), 3.15 (2H, t, J=5.6 Hz), 3.52 (2H, d, J=6.2 Hz), 6.01 (1H, t, J=6.2 Hz), 7.1-7.5 (6H, m), 7.6-7.8 (2H, m), 8.25 (1H, d, J=7.8 Hz) Mass (APCI): 360.07 (M⁺ + H) |
| (4) | H | 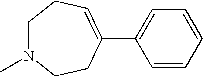 | ¹H NMR (200 MHz, CDCl₃, δ): 1.99 (2H, quint., J=5.3 Hz), 2.6-2.8 (2H, m), 2.8-3.1 (10H, m), 6.15 (1H, t, J=6.1 Hz), 7.2-7.5 (6H, m), 7.6-7.8 (2H, m), 8.25 (1H, d, J=7.4 Hz) Mass (APCI): 360.07 (M⁺ + H) |
| (5) | H | 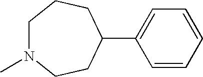 | ¹H NMR (200 MHz, CDCl₃, δ): 1.4-2.3 (10H, m), 2.8-3.1 (7H, m), 7.1-7.4 (5H, m), 7.42 (1H, t, J=7.9 Hz), 7.6-7.8 (2H, m), 8.28 (1H, d, J=7.8 Hz) Mass (APCI): 362.20 (M⁺ + H) |
| (6) | H | 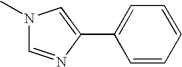 | ¹H NMR (200 MHz, DMSO-d₆, δ): 2.24 (2H, quint., J=7.2 Hz), 2.62(2H, t, J=7.4 Hz), 4.10 (2H, t, J=6.8 Hz), 7.18 (1H, t), 7.34 (2H, t, J=7.4 Hz), 7.46 (1H, t), 7.68 (1H, d), 7.7-7.9 (5H, m), 8.08 (1H, d, J=6.7 Hz), 12.19 (1H, br s) Mass (APCI): 331.07 (M⁺ + H) |
| (7) | H | 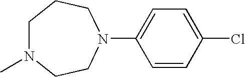 | ¹H NMR (200 MHz, DMSO-d₆, δ): 1.8-2.0 (4H, m), 2.3-2.7 (10H, m), 6.65 (2H, d, J=8 Hz), 7.02 (2H, d, J=8 Hz), 7.41 (1H, t, J=8 Hz), 7.61 (1H, d, J=8 Hz), 7.72 (1H, t, J=8 Hz), 8.08 (1H, d, J=8 Hz) Mass: 397 (M⁺ + H) |
| (8) | H | 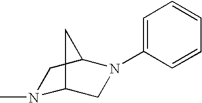 | ¹H NMR (200 MHz, DMSO-d₆, δ): 1.8-2.0 (2H, m), 2.4-3.2 (12H, m), 6.6-6.8 (2H, m), 6.8-7.0 (2H, m), 7.3-7.8 (3H, m), 8.06 (1H, m) Mass: 379 (M⁺ + H) |
| (9) | Cl | 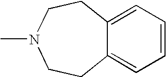 | ¹H NMR (200 MHz, DMSO-d₆, δ): 1.8-2.0 (2H, m), 2.3-2.8 (12H, m), 7.06 (4H, m), 7.39 (1H, t, J=8 Hz), 7.91 (1H, d, J=8 Hz), 8.02 (1H, d, J=8 Hz) Mass: 368 (M⁺ + H) |
| (10) | Me | 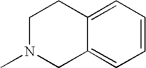 | ¹H NMR (200 MHz, DMSO-d₆, δ): 1.8-2.0 (2H, m), 2.52 (3h, s), 2.4-2.8 (10H, m), 7.1-7.3 (4H, m), 7.31 (1H, t, J=8 Hz), 7.62 (1H, d, J=8 Hz), 7.91 (1H, d, J=8 Hz) Mass: 334 (M⁺ + H) |
| (11) | H | 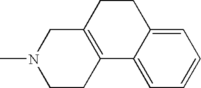 | ¹H NMR (200 MHz, CDCl₃, δ): 2.06 (2H, quint., J=6.4 Hz), 2.20 (2H, t, J=7.9 Hz), 2.65 (2H, t, J=6.2 Hz), 2.7-3.0 (8H, m), 3.20 (2H, br s), 7.1-7.3 (4H, m), 7.41 (1H, t, J=7.3 Hz), 7.63 (1H, d, J=6.9 Hz), 7.72 (1H, t, J=7.4 Hz), 8.22 (1H, d, J=7.8 Hz) Mass (API-ES): 372.3 (M⁺ + H) |
| (12) | H | 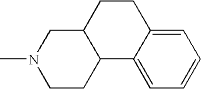 | ¹H NMR (200 MHz, CDCl₃, δ): 1.8-2.1 (4H, m), 2.1-2.4 (4H, m), 2.4-2.6 (3H, m), 2.8-2.9 (7H, m), 7.13 (4H, t, J=4.9 Hz), 7.42 (1H, t, J=6.8 Hz), 7.63 (1H, d, J=7.0 Hz), 7.72 (1H, t, J=6.8 Hz), 8.22 (1H, d, J=7.8 Hz) Mass (APCI): 373.87 (M⁺ + H) |
| (13) | H | 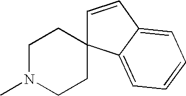 | ¹H NMR (200 MHz, CDCl₃, δ): 1.45 (2H, br d, J=14.7 Hz), 2.03 (2H, quint., J=5.5 Hz), 2.4-2.8 (6H, m), 2.9-3.1 (2H, m), 3.20 (2H, br d, J=11.5 Hz), 6.79 (1H, d, J=5.7 Hz), 6.91 (1H, d, J=5.7 Hz), 7.2-7.4 (3H, m), 7.45 (1H, t, J=6.6 Hz), 7.65 (1H, t, J=6.9 Hz), 7.73 (1H, t, J=6.8 Hz), 7.87 (1H, d, J=7.2 Hz), 8.33 (1H, d, J=7.9 Hz), 14.18 (1H, br) Mass (APCI): 372.07 (M⁺ + H) |
| (14) | H | 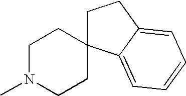 | ¹H NMR (200 MHz, CDCl₃, δ): 1.65 (2H, br s), 1.97 (2H, quint., J=5.4 Hz), 2.06 (2H, t, J=7.4 Hz), 2.2-2.6 (4H, m), 2.62 (2H, t, J=7.5 Hz), 2.8-3.1 (6H, m), 7.1-7.4 (3H, m), 7.43 (1H, t, J=8.1 Hz), 7.6-7.8 (3H, m), 8.31 (1H, d, J=7.9 Hz) Mass (APCI): 374.13 (M⁺ + H) |

### EXAMPLE 39

The following compounds are prepared in a similar manner to that of Example 9. If necessary, the starting compounds of them were prepared in similar manners of Preparation 17 and Preparation 20.

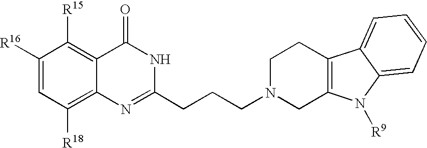

| No. | R¹⁵ | R¹⁶ | R¹⁸ | R²⁹ | |
|---|---|---|---|---|---|
| (1) | H | H | Cl | H | ¹H NMR (200 MHz, DMSO-d₆, δ): 2.0-2.2 (2H, m), 2.5-2.8 |
| | | | | | (6H, m), 6.95 (1H, t, J=8 Hz), 7.2-7.4 (4H, m), 7.79 (1H, d, J= |
| | | | | | 8 Hz), 7.95 (1H, d, J=8 Hz) |
| | | | | | Mass: 393 (M⁺ + H) |
| (2) | H | H | Me | H | ¹H NMR (200 MHz, DMSO-d₆, δ): 2.0-2.2 (2H, m), 2.5-2.8 |
| | | | | | (8H, m), 3.62 (2H, m), 6.8-7.4 (5H, m), 7.62 (1H, d, J=8 Hz), |
| | | | | | 7.90 (1H, d, J=8 Hz) |
| | | | | | Mass: 373 (M⁺ + H) |
| (3) | H | H | Me | Me | ¹H NMR (200 MHz, DMSO-d₆, δ): 2.0-2.2 (2H, m), 2.5-2.8 |
| | | | | | (8H, m), 2.52 (3H, s), 3.58 (3H, s), 6.8-7.4 (5H, m), 7.60 (1H, |
| | | | | | d, J=8 Hz), 7.88 (1H, d, J=8 Hz) |
| | | | | | Mass: 387 (M⁺ + H) |
| (4) | H | H | OMe | H | ¹H NMR (200 MHz, DMSO-d₆, δ): 2.0-2.2 (2H, m), 2.5-2.8 |
| | | | | | (8H, m), 3.89 (3H, s), 6.8-7.5 (6H, m), 7.62 (1H, d, J=8 Hz) |
| | | | | | Mass: 389 (M⁺ + H) |
| (5) | Cl | H | H | H | ¹H NMR (200 MHz, DMSO-d₆, δ): 2.0-2.2 (2H, m), 2.5-2.8 |
| | | | | | (6H, m), 3.0-3.2 (2H, m), 6.8-7.7 (7H, m) |
| | | | | | Mass: 393 (M⁺ + H) |
| (6) | H | Cl | H | H | ¹H NMR (200 MHz, DMSO-d₆, δ): 2.0-2.2 (2H, m), 2.5-2.8 |
| | | | | | (6H, m), 3.0-3.2 (2H, m), 6.8-7.3 (4H, m), 7.62 (1H, |
| | | | | | d, J=8 Hz), 7.78 (1H, dd, J=8, 1.2 Hz), 7.96 |
| | | | | | (1H, d, J=1.2 Hz) |
| | | | | | Mass: 393 (M⁺ + H) |

### EXAMPLE 40

The following compounds are prepared in a similar manner to that of Example 9. If necessary, the starting compounds of them were prepared in similar manners of Preparation 17 and Preparation 20.

(1) 2-[(1-ethyl-3-azetidinyl)methyl]-4(3H)-quinazolinone

¹H NMR (200 MHz, CDCl₃, δ): 1.04 (3H, t, J=7 Hz), 2.5-3.3 (9H, m), 7.4-8.2 (4H, m)

Mass: 244 (M⁺+H)

(2) 2-[(1-ethyl-3-pyrrolidinyl)methyl]-4(3H)-quinazolinone

¹H NMR (200 MHz, DMSO-d₆, δ): 1.06 (3H, t, J=8 Hz), 2.2-2.8 (7H, m), 7.4-8.2 (4H, m)

Mass: 258 (M⁺+H)

(3) 2-{[1-(3-phenylpropyl)-3-pyrrolidinyl]methyl}-4(3H)-quinazolinone

¹H NMR (200 MHz, DMSO-d₆, δ): 1.6-1.9 (2H, m), 2.1-2.8 (10H, m), 7.0-7.3 (5H, m), 7.48 (1H, t, J=8 Hz), 7.59 (1H, d, J=8 Hz), 7.75 (1H, t, J=8 Hz), 8.11 (1H, d, J=8 Hz)

Mass: 348(M⁺+H)

(4) 2-[(1-ethyl-4-piperidyl)methyl]-4(3H)-quinazolinone

¹H NMR (200 MHz, DMSO-d₆, δ): 0.95 (3H, t, J=7 Hz), 1.5-2.2 (4H, m), 2.32 (2H, q, J=7 Hz), 7.41 (1H, t, J=8 Hz), 7.52 (1H, d, J=8 Hz), 7.80 (1H, t J=8 Hz), 8.08 (1H, d, J=8 Hz)

Mass: 272 (M⁺+H)

(5) 2-{3-[4-ethynyl-3,6-dihydro-1(2H)-pyridinyl]propyl}-4(3H)-quinazolinone

¹H NMR (200 MHz, DMSO-d₆, δ): 1.7-2.2 (4H, m), 2.5-2.7 (2H, m), 2.7-2.9 (2H, m), 6.04 (1H, m), 7.40 (1H, t, J=8 Hz), 7.57 (1H, d, J=8 Hz), 7.75 (1H, t, J=8 Hz), 8.06 (1H, d, J=8 Hz)

Mass: 294 (M⁺+H)

(6) 2-{3-[4-phenylethynyl-3,6-dihydro-1(2H)-pyridinyl]propyl}-4(3H)-quinazolinone

¹H NMR (200 MHz, DMSO-d₆, δ): 1.8-2.0 (2H, m), 2.6-2.8 (4h, m), 3.78 (2H, s), 7.2-8.2 (11H, m)

Mass: 413 (M⁺+H)

(7) 2-{3-[4-(1-naphthylmethyl)-1-piperazinyl]propyl}-4(3H)-quinazolinone

¹H NMR (200 MHz, DMSO-d₆, δ): 1.7-2.0 (2H, m), 2.2-2.4 (2H, m), 2.5-2.8 (6H, m), 3.0-3.2 (2H, m), 6.12 (1H, m), 7.3-7.5 (6H, m), 7.59 (1H, d, J=8 Hz), 7.77 (1H, t, J=8 Hz), 8.06 (1H, d, J=8 Hz)

Mass: 370 (M⁺+H)

(8) 2-{3-[4-(ethylsulfonyl)-1-piperazinyl]propyl}-4(3H)-quinazolinone

¹H NMR (200 MHz, DMSO-d₆, δ): 1.14 (3H, t, J=7.5 Hz), 1.8-2.0 (2H, m), 2.5-2.8 (4H, m), 2.99 (2H, q, J=7.5 Hz), 3.0-3.3 (4H, m), 7.40 (1H, t, J=8 Hz), 7.52 (1H, d, J=8 Hz), 7.75 (1H, t, J=8 Hz), 8.09 (1H, d, J=8 Hz)

Mass: 365 (M⁺+H)

(9) 2-{3-[4-(2-furoyl)-1-piperazinyl]propyl}-4(3H)-quinazolinone

¹H NMR (200 MHz, DMSO-d₆, δ): 1.8-2.0 (2H, m), 2.2-2.8 (8H, m), 6.6-6.7 (1H, m), 6.9-7.0 (1H, m), 7.48 (1H, t, J=8 Hz), 7.68 (1H, d, J=8 Hz), 7,7-7.9 (2H, m), 8.09 (1H, m)

Mass: 367 (M⁺+H)

(10) 2-[3-(4-benzoyl-1-piperidyl)propyl]-4(3H)-quinazolinone

¹H NMR (200 MHz, DMSO-d₆, δ): 1.4-3.0 (15H, m), 7.4-7.9 (6H, m), 7.92 (2H, d, J=8 Hz), 8.06 (1H, d, J=8 Hz)

Mass: 376 (M⁺+H)

(11) 2-[3-(4-Phenyl-3,6-dihydro-1(2H)-pyridinyl)butyl]-4(3H)-quinazolinone

Mass (ESI): 360.3 (M⁺+H)

### EXAMPLE 41

The following compounds are prepared in a similar manner to that of Example 25. If necessary, the starting compounds of them were prepared in similar manners of Preparation 17, Preparation 20, preparation 23-(2) and Example 9.

(1) 2-(3-azetidinylmethyl)-4(3H)-quinazolinone hydrochloride

¹H NMR (200 MHz, DMSO-d₆, δ): 2.8-3.8 (5H, m), 7.4-8.2 (4H, m)

Mass: 202 (M⁺+H)

(2) 2-(3-pyrrolidinylmethyl)-4(3H)-quinazolinone hydrochloride

¹H NMR (200 MHz, DMSO-d₆, δ): 1.6-1.9 (2H, m), 2.0-2.2 (2H, m), 2.3-3.3 (5H, m), 7.5-8.3 (4H, m)

Mass: 230 (M⁺+H)

(3) 2-(4-piperidylmethyl)-4(3H)-quinazolinone hydrochloride

¹H NMR (200 MHz, DMSO-d₆, δ): 1.5-2.3 (5H, m), 2.6-3.2 (6H, m), 7.5-8.0 (3H, m), 8.15 (1H, d, J=8 Hz)

Mass: 244 (M⁺+H)

### EXAMPLE 42

2-{[5-[(Benzyloxy)carbonylamino]hexanoyl]amino}benzamide (2.8 g, 7.3 mmol) was dissolved in 1N NaOH (36.5 mL) and dioxane. The reaction mixture was stirred at room temperature for 2 hours. The mixture was acidified with 6N HCl aqueous solution and extracted with AcOEt, washed with brine. The organic layer was dried over MgSO₄ and the solvent was removed in vacuo. The obtained powder was washed with ether to give 2-{5-[(benzyloxy)carbonylamino]pentyl}-4(3H)-quinazolinone as colorless powder (1.99 g, 5.4 mmol, 75%)

¹H NMR (300 MHz, CDCl₃, δ): 1.48(2H, t, J=7.9 Hz), 1.60 (2H, m), 1.89 (2H, quint. J=7.8 Hz), 2.74 (2H, t, J=7.6 Hz), 3.25 (2H, t, J=6.7 Hz), 4.86 (1H, br.s), 5.09 (2H, s), 7.39 (5H, m), 7.45 (1H, t, J=7.3 Hz), 7.69 (2H, m), and 8.26 (1H, d, J=6.9 Hz)

Mass (m/z): 366(M⁺+1)

### EXAMPLE 43

2-{5-[(Benzyloxy)carbonylamino]pentyl}-4(3H)-quinazolinone (500 mg, 1.37 mmol) and 10% Pd-C (50 mg) was suspended in THF/MeOH (1:1, 20 mL). The mixture was hydrogenated at 3 atm of hydrogen for 8 hours. After filtration of Pd-C, the solvent was removed in vacuo. The residue was washed with methanol and ether to give 2-(5-aminopentyl)-4(3H)-quinazolinone (136 mg, 0.59 mmol, 43%) as colorless powder.

¹H NMR (300 MHz, CDCl₃, δ): 1.36 (4H, s), 1.71 (2H, s), 2.51 (4H, s), 7.44 (1H, d, J=7.0 Hz), 7.58 (1H, d, J=8.5 Hz), 7.76 (1H, t, J=7.7 Hz), and 8.07 (1H, d, J=7.7 Hz)

### EXAMPLE 44

To a solution of 2-(5-aminopentyl)-4(3H)-quinazolinone (100 mg, 0.432 mmol) in ethanol (5 mL) benzamide (45.9 mg, 0.432 mmol) was added. After stirring for 30 minutes at room temperature, sodium brohydride was added to the mixture, and the mixture was stirred at room temperature for 4 hours.

The reaction mixture was extracted with AcOEt and washed with saturated sodium hydrogen carbonate aqueous solution and brine. The organic layer was dried over MgSO₄, and the solvent was removed in vacuo. The residual colorless powder was purified with preparative TLC to give 2-(N-benzyl-5-aminopentyl)-4(3H)-quinazolinone (24 mg, 0.075 mmol, 17%) as colorless powder.

¹H NMR (300 MHz, CDCl₃, δ): 1.50 (1H, m), 1.61 (1H, m), 1.88 (2H, quint, J=7.6 Hz), 2.66 (2H, t, J=7.0 Hz), 2.75 (2H, t, J=7.7 Hz), 3.79 (2H, s), 7.25-7.32 (5H, m), 7.45 (1H, t, J=8.0 Hz), 7.68 (1H, t, J=8.1 Hz), 7.76 (1H, t, J=7.0 Hz), and 8.27 (1H, d, J=6.5 Hz)

Mass (m/z): 322 (M⁺+1)

### EXAMPLE 45

The following compounds are prepared in a similar manner to those of Preparation 31, Example 42 and Example 43.

(1) 2-(3-aminopropyl)-4(3H)-quinazolinone

¹H NMR (200 MHz, DMSO-d₆, δ): 1.8-2.0 (2H, m), 2.4-3.3 (4H, m), 7.2-8.2 (4H, m)

Mass: 204 (M⁺+H)

(2) 2-(3-aminoethyl)-4(3H)-quinazolinone

¹HNMR (200 MHz, DMSO-d₆, δ): 2.4-2.9 (4H, m), 7.2-8.2 (4H, m)

Mass: 190 (M⁺+H)

(3) 2-(3-aminomethyl)-4(3H)-quinazolinone

¹H NMR (200 MHz, DMSO-d₆, δ): 7.2-8.2 (4H, m)

Mass: 176 (M⁺+H)

### EXAMPLE 46

The following compounds are prepared in a similar manner to those of Preparation 31, Example 42, Example 43 and Example 25.

(1) 2-[(1E)-3-amino-3-methyl-1-butenyl]-4(3H)-quinazolinone hydrochloride

¹H NMR (200 MHz, DMSO-d₆, δ): 1.41 (3H, s), 1.64 (3H, s), 6.50 (1H, d, J=16 Hz), 7.22 (1H, d, J=16 Hz), 7.3-8.3 (4H, m)

Mass: 230 (M⁺+H)

### EXAMPLE 47

The following compounds are prepared in a similar manner to those of Preparation 31, Example 42, Example 43 and Example 44.

(1) 2-{3-[methyl(3-phenylpropyl)amino]propyl}-4(3H)-quinazolinone

¹H NMR (200 MHz, DMSO-d₆, δ): 1.6-2.0 (4H, m), 2.20 (3H, m), 2.2-2.8 (8H, m), 7.0-8.0(8H, m)

Mass: 336 (M⁺+H)

(2) 2-{3-[(4-phenylbutyl)amino]propyl}-4(3H)-quinazolinone

¹H NMR (200 MHz, DMSO-d₆, δ): 1.2-1.8 (8H, m), 2.3-2.6 (6H, m), 7.0-7.8 (9H, m), 8.07 (1H,d, J=8 Hz)

Mass: 336 (M⁺+H)

(3) 2-{3-[(3-phenylpropyl)amino]propyl}-4(3H)-quinazolinone

¹H NMR (200 MHz, DMSO-d₆, δ): 1.6-2.0 (4H, m), 2.3-2.7 (8H, m), 7.0-7.8 (8H, m), 8.07 (1H, d, J=8 Hz)

Mass: 322 (M⁺+H)

(4) 2-{3-[(2-phenylethyl)amino]propyl}-4(3H)-quinazolinone

¹H NMR (200 MHz, DMSO-d₆, δ): 1.6-2.0(2H, m), 2.3-2.7 (8H, m), 7.0-7.8 (8H, m), 8.08 (1H, d, J=8 Hz)

Mass: 308 (M⁺+H)

(5) 8-methyl-2-{3-[(3-phenylpropyl)amino]propyl}-4(3H)-quinazolinone

¹H NMR (200 MHz, DMSO-d₆, δ): 1.6-2.0 (4H, m), 2.45 (3H, s), 2.4-2.7 (8H, m), 7.0-7.4 (6H, m), 7.62 (1H, d, J=8 Hz), 7.89 (1H, d, J=8 Hz)

Mass: 336 (M⁺+H)

(6) 2-{3-[(4-phenoxybenzyl)amino]propyl}-4(3H)-quinazolinone

¹H NMR (200 MHz, DMSO-d₆, δ): 1.8-2.0 (2H, m), 2.4-2.8 (4H, m), 3.66 (2H, s), 6.8-7.8 (13H, m), 8.08 (1H, d, J=8 Hz)

Mass: 386 (M⁺+H)

(7) 2-{3-[(1,1′-biphenyl-3-ylmethyl)amino]propyl}-4(3H)-quinazolinone

¹H NMR (200 MHz, DMSO-d₆, δ): 1.8-2.0 (2H, m), 2.4-2.8 (4H, m), 3.72 (2H, s), 7.2-7.8 (12H, m), 8.06 (1H, d, J=8 Hz)

Mass: 370 (M⁺+H)

(8) 2-{3-[(1,1′-biphenyl-2-ylmethyl)amino]propyl}-4(3H)-quinazolinone

¹H NMR (200 MHz, DMSO-d₆, δ): 1.8-2.0 (2H, m), 2.4-2.8 (4H, m), 3.72 (2H, s), 7.2-7.8 (12, m), 8.06 (1H, d, J=8 Hz)

Mass: 370 (M⁺+H)

(9) 2-{3-[(1,1′-biphenyl-4-ylmethyl)amino]propyl}-4(3H)-quinazolinone

¹H NMR (200 MHz, DMSO-d₆, δ): 1.8-2.0 (2H, m), 2.4-2.9 (4H, m), 3.76 (2H, s), 7.2-7.8 (12H, m), 8.06 (1H, d, J=8 Hz)

Mass: 370 (M⁺+H)

### EXAMPLE 48

The following compounds are prepared in a similar manner to those of Preparation 31, Example 42, Example 43, Example 44 and Example 25.

(1) 2-{3-[(1H-benzimidazol-2-ylmethyl)amino]propyl}-4(3H)-quinazolinone dihydrochloride

¹H NMR (200 MHz, DMSO-d₆, δ): 2.2-2.9 (4H, m), 4.72 (2H, s), 7.2-7.8 (6H, m), 8.0-8.2 (2H, m), 8.2-8.3 (1H, m)

Mass: 334 (M⁺+H)

### EXAMPLE 49

The following compounds are prepared in a similar manner to that of Preparation 31, Example 42, Example 43 and Example 44.

(1) 2-[3-(diethylamino)propyl]-4(3H)-quinazolinone

¹H NMR (200 MHz, DMSO-d₆, δ): 0.94 (6H, t, J=7.4 Hz), 1.8-2.0 (2H, m), 2.3-2.7 (8H, m), 7.44 (1H, t, J=8.2 Hz), 7.57 (1H, d, J=8.2 Hz), 7.76 (1H, d, J=8.2 Hz), 8.06 (1H, d, J=8.2 Hz)

Mass: 260 (M⁺+H)

(2) 2-[3-(2,3-dihydro-1H-inden-2-ylamino)propyl]-4(3H)-quinazolinone

¹H NMR (200 MHz, DMSO-d₆, δ): 1.8-2.0 (2H, m), 2.4-3.0 (9H, m), 6.8-8.0 (8H, m)

(3) 2-[3-(2,3-dihydro-1H-inden-2-ylamino)propyl]-8-methyl-4(3H)-quinazolinone

¹H NMR (200 MHz, DMSO-d₆, δ): 1.8-2.0 (2H, m), 2.51 (3H s), 2.6-2.8 (4H, m), 7.1-7.3 (4H, m), 7.29 (1H, t, J=8 Hz), 7.62 (1H, d, J=8 Hz), 7.91 (1H, d, J=8 Hz)

Mass: 334 (M⁺+H)

(4) 2-{3-[2,3-dihydro-1H-inden-2-yl(methyl)amino]propyl}-4(3H)-quinazolinone

¹H NMR (200 MHz, DMSO-d₆, δ): 1.8-2.0 (2H, m), 2.18 (3H, s), 2.2-3.3 (9H, m), 7.0-7.2 (4H, m), 7.38 (1H, t, J=8 Hz), 7.58 (1H, d, J=8 Hz), 7.78 (1H, t, J=8 Hz), 8.05 (1H, d, J=8 Hz)

1. A compound of the formula: 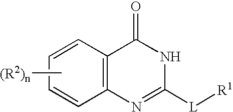
wherein R¹ is optionally substituted cyclic amino groups or optionally substituted amino group,
R² is substituent,
n means an integer from 0 to 4, and
L is lower alkylene or lower alkenylene,
or its prodrug, or their salts:

2. The compound according to claim 1, wherein
R² is halogen, nitro, amino, acylamino, aryl(lower)alkylamino, lower alkylamino, lower alkyl, lower alkynyl, lower alkoxy, acyl, or cyclic amino group optionally substituted with lower alkyl.

3. The compound according to claim 2, wherein
R¹ is (1) cyclic amino group optionally substituted with one or more substituent(s) selected from the group consisting of halogen, cyano, hydroxy, amino, oxo, lower alkyl, lower alkenyl, lower alkynyl, aryl(lower)alkyl, aryl(lower)alkynyl, acyl, lower alkylsulfonyl, optionally substituted heteroaryl and optionally substituted aryl, or (2) amino optionally substituted with 1 or 2 substituent(s) selected from the group consisting of lower alkyl, aryl, heteroaryl(lower)alkyl, aryl(lower)alkoxycarbonyl and aryl(lower)alkyl optionally substituted with aryl or aryloxy.

4. The compound according to claim 3, wherein
R¹ is cyclic amino group optionally substituted with optionally substituted heteroaryl or optionally substituted aryl.

5. The compound according to claim 4, wherein
R¹ is cyclic amino group with saturated or unsaturated monocyclic group with one or more nitrogen atom(s), which is substituted with optionally substituted heteroaryl or optionally substituted aryl.

6. The compound according to claim 5, wherein
R¹ is tetrahydropyridyl, piperidyl or piperazinyl, each of which is substituted with optionally substituted heteroaryl or optionally substituted aryl.

7. The compound according to any one of claims 4, 5 and 6, wherein
substituent(s) of optionally substituted heteroaryl is lower alkyl, halogen, cyano or acyl, or
substituent(s) of optionally substituted aryl is halogen, cyano, hydroxy, carboxy, nitro, amino, lower alkyl, hydroxy(lower)alkyl, lower alkoxy, lower alkylthio, halo(lower)alkyl, lower alkylamino, acylamino, halo(lower)alkoxy, aryl, aryloxy, or acyl.

8. The compound according to claim 3, wherein
R¹ is cyclic amino groups with saturated and unsaturated fused cyclic groups, which is substituted with optionally substituted lower alkyl.

9. The compound according to any one of claims 4, 5, 6, 7 and 8, wherein L is trimethylene.

10. The compound according to claim 9, which is selected from the group consisting of:
(1) 5-chloro-2-[3-(4-phenyl-3,6-dihydro-1(2H)-pyridinyl)propyl]-4(3H)-quinazolinone,
(2) 2-{3-[4-(4-hydroxyphenyl)-3,6-dihydropyridin-1(2H)-yl]propyl}-4(3H)-quinazolinone,
(3) 8-methyl-2-{3-[4-(4-methoxyphenyl)-3,6-dihydro-1(2H)-pyridinyl]propyl}-4(3H)-quinazolinone,
(4) 8-chloro-2-{3-[4-(4-fluorophenyl)-3,6-dihydro-1(2H)-pyridinyl]propyl}-4(3H)-quinazolinone,
(5) 8-chloro-2-{(1E)-3-[4-(4-fluorophenyl)-3,6-dihydro-1(2H)-pyridinyl]-1-propenyl}-4(3H)-quinazolinone,
(6) 8-Chloro-2-{[4-(4-pyridinyl)-3,6-dihydro-1(2H)-pyridinyl]propyl}-4(3H)-quinazolinone,
(7) 2-{3-[4(4-chlorophenyl)-1-piperazinyl]propyl}-4(3H)-quinazolinone,
(8) 2-{3-[4-(4-pyridyl)-1-piperazinyl]propyl}-4(3H)-quinazolinone,
(9) 2-[3-(1,4,5,6-Tetrahydrobenzo[f]isoquinolin-3(2H)-yl)propyl]-4(3H)-quinazolinone, and
(10) 8-methyl-2-[3-(1,3,4,9-tetrahydro-2H-pyrido[3,4-b]indol-2-yl)propyl]-4(3H)-quinazolinone.

11. A process for preparing a compound of the formula: 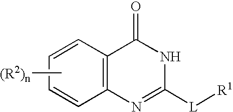
wherein R¹ is optionally substituted cyclic amino groups or optionally substituted amino group,
R² is substituent,
n means an integer from 0 to 4, and
L is lower alkylene or lower alkenylene,
or its prodrug, or their salts,
which comprises,
(1) reacting the formyl group of the compound (II) of the formula: 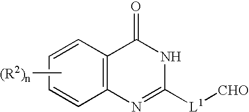
or its aminal derivative, or their salt, and imino group of the compound (IV) of the formula:
R¹—H
or its salt, in the presence of a reducing agent to provide a compound of the formula: 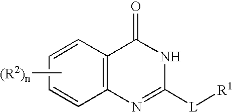
or its salt, in the above formulae,
R¹, R², n and L are each as defined above, and L¹ is lower alkylene or lower alkenylene delating a methylene group from the end of the one defined in L, or
(2) subjecting the compound (III) of the following formula: 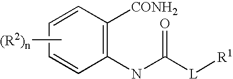
or its salt, to cyclization reaction in the presence of base to provide a compound of the formula: 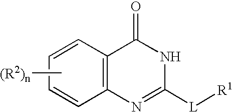
or its salt, in the above formurae,
R¹, R², n and L are each as defined above.

12. A pharmaceutically composition comprising a compound of the formula: 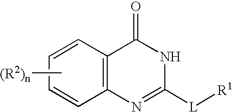
wherein R¹ is optionally substituted cyclic amino groups or optionally substituted amino group,
R² is substituent,
n means an integer from 0 to 4, and
L is lower alkylene or lower alkenylene,
or its prodrug, or their pharmaceutically acceptable salts, and a pharmaceutically acceptable carrier, wherein said compound is present in an amount effective for inhibiting PARP activity.

13. The pharmaceutical composition of claim 12 for treating or preventing diseases ascribed by NMDA- and NO-induced toxicity.

14. The pharmaceutical composition of claim 12 for extending the lifespan or proliferative capacity of cells or altering gene expression of senescent cells

15. The pharmaceutical composition of claim 13 for treating or preventing tissue damage resulting from cell damage or death due to necrosis or apoptosis; neural tissue damage resulting from ischemia and reperfusion injury, neurological disorders and neurodegenerative diseases; neurodegenerative diseases; head trauma; stroke; Alzheimer's disease; Perkinson's disease; epilepsy; Amyotrophic Lateral Scleosis (ALS); Huntington's disease; schizopherenia; chronic pain; ischemia and nloss following hypoxia; hypoglycemia; ischemia; trauma; nervous insult; previously ischemic heart or skeleton muscle tissue; radiosensitizing hypoxic tumor cells; tumor cells from recovering from potentially lethal damage of DNA after radiation therapy; skin aging; atheroscleosis; osteoarthritis; osteoporosis; muscular dystrophy; degenerative diseases of skeletal muscle involving replicative senescence; age-related macular degeneration; immune senescence; AIDS; and other immune senescencediseases; inflammatory bowel disorders (e.g., colitis); arthritis; diabetes; endotoxic shock; septic shock; and tumor.

16. A method of inhibiting PARP activity comprising administering a compound of the formula: 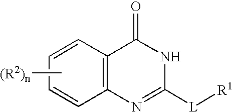
wherein R¹ is optionally substituted cyclic amino groups or optionally substituted amino group,
R¹ is substituent,
n means an integer from 0 to 4, and
L is lower alkylene or lower alkenylene,
or its prodrug, or their pharmaceutically acceptable salts, and a pharmaceutically acceptable carrier, wherein said compound is present in an amount effective for inhibiting PARP activity.
